SEQUENCE LISTING (96 sequences)

SEQ ID NO: 1 - protein, 283 aa, Homo sapiens
LRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSG

SEQ ID NO: 2 - protein, 275 aa, Mus musculus
LQCMQCESNQSCLVEECALGQDLCRTTVLREWQDDRELEVVTRGCAHSEKTNRTMSYRMGSMIISLTETVCATNLCNRPRPGARGRAFPQGRYLECASCTSLDQSCERGREQSLQCRYPTEHCIEVVTLQSTERSLKDQDYTRGCGSLPGCPGTAGFHSNQTFHFLKCCNYTHCNGGPVLDLQSFPPNGFQCYSCEGNNTLGCSSEEASLINCRGPMNQCLVATGLDVLGNRSYTVRGCATASWCQGSHVADSFPTHLNVSVSCCHGSGCNSPTG

SEQ ID NO: 3 - protein, 48 aa, Homo sapiens
SNELHQVPSNCDCLNGGTCVSNKYFSNIHWCNCPKKFGGQHCEIDKSK

SEQ ID NO: 4 - protein, 49 aa, Mus musculus
GSVLGAPDESNCGCQNGGVCVSYKYFSRIRRCSCPRKFQGEHCEIDASK

SEQ ID NO: 5 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 6 - protein, 226 aa, Mus musculus
PRDCGCKPCICTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 7 - protein, 8 aa, Artificial Sequence
  Linker sequence
GGAGAAGG

SEQ ID NO: 8 - protein, 8 aa, Artificial Sequence
  Linker sequence
GGAGAAGG

SEQ ID NO: 9 - protein, 20 aa, Artificial Sequence
  Linker sequence
LEVLFQGPLELEVLFQGPIE

SEQ ID NO: 10 - protein, 14 aa, Artificial Sequence
  Linker sequence
LEVLFQGPLEAGAG

SEQ ID NO: 11 - protein, 12 aa, Artificial Sequence
  Linker sequence
VELEVLFQGPIE

SEQ ID NO: 12 - protein, 583 aa, Artificial Sequence [sequence omitted]

SEQ ID NO: 13 - protein, 573 aa, Artificial Sequence
  GFDuPAR-mFc
SNELHQVPSNCDCLNGGTCVSNKYFSNIHWCNCPKKFGGQHCEIDKSKGGAGAAGGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDLEVLFQGPLEAGAGPRDCGCKPCICTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 14 - protein, 528 aa, Artificial Sequence
  uPAR-hFc
LRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDLEVLFQGPLELEVLFQGPIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 15 - protein, 517 aa, Artificial Sequence
  uPAR-mFc
LRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDLEVLFQGPLEAGAGPRDCGCKPCICTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 16 - protein, 339 aa, Artificial Sequence
  GFDuPAR
SNELHQVPSNCDCLNGGTCVSNKYFSNIHWCNCPKKFGGQHCEIDKSKGGAGAAGGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSG

SEQ ID NO: 17 - protein, 573 aa, Artificial Sequence
  mGFDmuPAR-Fc
GSVLGAPDESNCGCQNGGVCVSYKYFSRIRRCSCPRKFQGEHCEIDASKGGAGAAGGLQCMQCESNQSCLVEECALGQDLCRTTVLREWQDDRELEVVTRGCAHSEKTNRTMSYRMGSMIISLTETVCATNLCNRPRPGARGRAFPQGRYLECASCTSLDQSCERGREQSLQCRYPTEHCIEVVTLQSTERSLKDEDYTRGCGSLPGCPGTAGFHSNQTFHFLKCCNYTHCNGGPVLDLQSFPPNGFQCYSCEGNNTLGCSSEEASLINCRGPMNQCLVATGLDVLGNRSYTVRGCATASWCQGSHVADSFPTHLNVSVSCCHGSGCNSPVELEVLFQGPIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 18 - protein, 112 aa, Artificial Sequence
  Heavy-chain variable region of 8B12
ELMKPGASVKLSCKAAGYTFTAYWIEWIRQRPGHGLEWIGEILPGSSSTNCNEMFKGKATFTADTSSNSAYMQLSSLTTEDSAIYYCTRDFSGDRSNLYFDVWGTGTTVTVS

SEQ ID NO: 19 - protein, 112 aa, Artificial Sequence
  Heavy-chain variable region of 10H6
ELMKPGASVKLSCKAAGYTFTAYWIEWIRQRPGHGLEWIGEILPGSSSSNCNEMFKGKATFTADTSSNSAYMQLSSLTTEDSAIYYCTRDFSGDRSNLYFDVWGTGTTVTVS

SEQ ID NO: 20 - protein, 112 aa, Artificial Sequence
  Heavy-chain variable region of 19.10
ELVKPGASVKLSCKATGYTFTVYWMEWVRQRPGHGLEWIGEILPGSGRTNYNERFKGKATFTADTSSNTAYMQLSSLTTEDSAIYYCARDYSGDTSTGYFDVWGTGTTVTVS

SEQ ID NO: 21 - protein, 112 aa, Artificial Sequence
  Heavy-chain variable region of 13D11
ELMKPGASVKLSCKATGYTFTDYWIEWVKQRPGHGLEWIGEILPGRDSTDYNENFKGKATFTADTSSNTAYMQLSSLTTEDSAIYYCARDYYGHISNWYFDVWGTGTTVTVS

SEQ ID NO: 22 - protein, 5 aa, Artificial Sequence
  CDR3 VH of 3B6
EWDPA

SEQ ID NO: 23 - protein, 8 aa, Artificial Sequence
  CDR3 VH of 3C10
KTKGLPHF

SEQ ID NO: 24 - protein, 111 aa, Artificial Sequence
  Heavy-chain variable region AL6
ELMKPGASVKLSCKATGYSFTGYWIEWVKQRPGHGLEWIGEILPGSDRTNYNEKFKGKATFTADTSSNTAYMQLSSLTTEDSAIYYCARDDYDRGLYIMDYWGQGTSVTVS

SEQ ID NO: 25 - protein, 113 aa, Artificial Sequence
  Heavy chain variable regions of OMD4
VLVKPGASVKMSCKASGYTFTDYYMHWVKQSHGKSLEWIGIINPYNGGTSYNQKFKDKATLTVDKSSSTAYMELNSLTSEDSAVYYCARSGYDYDGSYGMDYWGQGTSVTVSS

SEQ ID NO: 26 - protein, 286 aa, Artificial Sequence
  uPARmyc
LRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPGGEQKLISEEDL

SEQ ID NO: 27 - protein, 12 aa, Artificial Sequence
  C-terminal myc-tag
GGEQKLISEEDL

SEQ ID NO: 28 - protein, 342 aa, Artificial Sequence
  GFDuPARmyc
SNELHQVPSNCDCLNGGTCVSNKYFSNIHWCNCPKKFGGQHCEIDKSKGGAGAAGGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPGGEQKLISEEDL

SEQ ID NO: 29 - DNA, 31 bp, Artificial Sequence
  synthetic oligonucleotide
gtaaatgagcggccgcgtcgagtctagaggg

SEQ ID NO: 30 - DNA, 30 bp, Artificial Sequence
  synthetic oligonucleotide
ccctctagactcgacgcggccgctcattta

SEQ ID NO: 31 - DNA, 30 bp, Artificial Sequence
  synthetic oligonucleotide
tcgagctggaagttctgttccaggggccca

SEQ ID NO: 32 - DNA, 30 bp, Artificial Sequence
  synthetic oligonucleotide
agctacccggggaccttgtcttgaaggtcg

SEQ ID NO: 33 - DNA, 24 bp, Artificial Sequence
  synthetic oligonucleotide
cggggtaccatggcacccctgaga

SEQ ID NO: 34 - DNA, 28 bp, Artificial Sequence
  synthetic oligonucleotide
ttgcggccgctcatttacccggagacag

SEQ ID NO: 35 - DNA, 40 bp, Artificial Sequence
  synthetic oligonucleotide
gcctcgaggcaggagcaggacccagggattgtggttgtaa

SEQ ID NO: 36 - DNA, 28 bp, Artificial Sequence
  synthetic oligonucleotide
gcgcggccgctcatttaccaggagagtg

SEQ ID NO: 37 - DNA, 42 bp, Artificial Sequence
  synthetic oligonucleotide
gcgtcgacggtacccgccaccatgggtcacccgccgctgctg

SEQ ID NO: 38 - DNA, 59 bp, Artificial Sequence
  synthetic oligonucleotide
gcctcgaggggcccctggaacagaacttccagatccaggtctgggtggttacagccact

SEQ ID NO: 39 - DNA, 67 bp, Artificial Sequence
  synthetic oligonucleotide
gcgcggccgctcacagatcctcttcagagatgagtttctgctctcctcctgggtggttacagccact

SEQ ID NO: 40 - DNA, 36 bp, Artificial Sequence
  synthetic oligonucleotide
gcggtacccgccaccatgagagccctgctggcgcgc

SEQ ID NO: 41 - DNA, 36 bp, Artificial Sequence
  synthetic oligonucleotide
tgtgaaatagataagtcaaaagggggggccggggcg

SEQ ID NO: 42 - DNA, 45 bp, Artificial Sequence
  synthetic oligonucleotide
gggggggccggggcggctggaggactgcggtgcatgcagtgtaag

SEQ ID NO: 43 - DNA, 33 bp, Artificial Sequence
  synthetic oligonucleotide
tagtttagcggccgcttaggtccagaggagagt

SEQ ID NO: 44 - DNA, 59 bp, Artificial Sequence; identical to SEQ ID NO: 38
  synthetic oligonucleotide

SEQ ID NO: 45 - DNA, 67 bp, Artificial Sequence; identical to SEQ ID NO: 39
  synthetic oligonucleotide

SEQ ID NO: 46 - DNA, 36 bp, Artificial Sequence
  synthetic oligonucleotide
gggggggccggggcgctgcggtgcatgcagtgtaag

SEQ ID NO: 47 - DNA, 69 bp, Artificial Sequence
  synthetic oligonucleotide
gggggggccggggcggctggagcaggagcaggtgctggtgctggaggactgcggtgcatgcagtgtaag

SEQ ID NO: 48 - DNA, 81 bp, Artificial Sequence
  synthetic oligonucleotide
gggggggccggggcggctggagcaggagcaggtgctggtgctggagcaggtgctggtggtctgcggtgcatgcagtgtaag

SEQ ID NO: 49 - DNA, 36 bp, Artificial Sequence
  synthetic oligonucleotide
ggaagatctatagacagatgggggtgtcgttttggc

SEQ ID NO: 50 - DNA, 32 bp, Artificial Sequence
  synthetic oligonucleotide
cttccggaattcnangtnnagctgnagnagtc

SEQ ID NO: 51 - DNA, 35 bp, Artificial Sequence
  synthetic oligonucleotide
cttccggaattcnangtnnagctgnagnagtcngg

SEQ ID NO: 52 - DNA, 30 bp, Artificial Sequence
  synthetic oligonucleotide
ggtgcatgcggatacagttggtgcagcatc

SEQ ID NO: 53 - DNA, 32 bp, Artificial Sequence
  synthetic oligonucleotide
gggagctcganattgtgntnacncannctnca

SEQ ID NO: 54 - DNA, 28 bp, Artificial Sequence
  synthetic oligonucleotide
ggggtaccatgaaagtctggctggcgag

SEQ ID NO: 55 - DNA, 36 bp, Artificial Sequence
  synthetic oligonucleotide
cgccccggcccctccttttgatgcatctatctcaca

SEQ ID NO: 56 - DNA, 45 bp, Artificial Sequence
  synthetic oligonucleotide
ggaggggccggggcggctggaggactgcagtgcatgcagtgtgag

SEQ ID NO: 57 - DNA, 26 bp, Artificial Sequence
  synthetic oligonucleotide
agcggctgtaacagccccgtcgaccg

SEQ ID NO: 58 - protein, 335 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 59 - DNA, 5 bp, Artificial Sequence
  linker
ggaga

SEQ ID NO: 60 - DNA, 16 bp, Artificial Sequence
  linker
ggagaagagagagagg

SEQ ID NO: 61 - DNA, 20 bp, Artificial Sequence
  linker
ggagaagagagagagagagg

SEQ ID NO: 62 - protein, 108 aa, Artificial Sequence
  Heavy-chain variable region 13F6
ELVKPGASVELSCKASGYTFTSYWMNWVKQRPGRGLEWIGRIAPNSGGTQYSGKFKTKATLTVDKSSSTAYMQLSSLTSEDSAVYYCARGDYFGHFDYWGQGTTLTVS

SEQ ID NO: 63 - protein, 106 aa, Artificial Sequence
  Heavy-chain variable region AL38
XXXXXGSSMKLSCTVSGFTFSDYYMAWVRQVPEKGLEWVANINYDGSKSYYLDSLRSRFIISRDNAKNILYLHMSSLKSEDTATYYCSREGYAMDYWGQGTSVTVS

SEQ ID NO: 64 - protein, 109 aa, Artificial Sequence
  Heavy-chain variable region BE18
ELVKPGASVKIICKASGYIFTDYNMDWVKQSHGKSLEWIGEINPNNGDTIYNQNFKGKATLTVDKSSSTAHMALRSLTSEDTAVYYCTRKGGLRAWFVYWGQGTLVTVS

SEQ ID NO: 65 - protein, 100 aa, Artificial Sequence
  Light-chain variable region 10H6
XXXXXXXXXXSLTCRASQDIGSSLNWLQQEPDGTIKRLIYATSSLDSGVPKRFSGSRSGSDYSLTISRLESEDFVDYYCLQYATSPYTFGGGTKLEIKXX

SEQ ID NO: 66 - protein, 100 aa, Artificial Sequence
  Light-chain variable region 8B12
SLSASLGERVSLTCRASQDIGSSLNWLQQEPDGTIKRLIYATSSLDSGVPKRFSGSRSGSDYSLTISRLESEDFVDYYCLQYATSPYTFGGGTKLEIKRA

SEQ ID NO: 67 - protein, 100 aa, Artificial Sequence
  Light-chain variable region 13D11
SLSASLGERVSLTCRASQDIGSSLNWLQQEPDGTIKRLIYATSSLDSGVPKRFSGSRSGSDYSLTISSLESVDFVVYYCLQYATSPYTFGGGTKLEIKRA

SEQ ID NO: 68 - protein, 100 aa, Artificial Sequence
  Light-chain variable region 19.10
SLSASLGERVSLTCRASQDIGSGLSWLQQEPDGTIKRLIYATSSLDSGVPKRFSGSRSGSDYSLTISSLESEDFVDYYCLQYATSPYTFGGGTKLEIKRA

SEQ ID NO: 69 - protein, 100 aa, Artificial Sequence
  Light-chain variable region AL6
SLSASLGERVSLTCRASQDIGTSLNWLQQEPDGTIRRLIYATSSLPPGVPKRFSGSRSGSDYSLTISSLESEDFVNYYCLQYATSPYTFGGGTKLEIKRA

SEQ ID NO: 70 - protein, 99 aa, Artificial Sequence
  Light-chain variable region 13F6
SLSASLGGKVTITCKASQDINNYIAWYQHKPGKGPRLLIHYTSILQPGIPSRFSGSGSGRDYSFSISNLEPEDIATYYCLQCDNLLTFGAGTKLELKRA

SEQ ID NO: 71 - protein, 101 aa, Artificial Sequence
  Light-chain variable region BE18
XMSASPGEKVTMTCRASSSVSSSYLHWYQQKSGASPKVWIYSTSNLASGVPARFSGSGSGTSYSLTISSVEAEDAATYYCQQYSGYPLTFGSGTKLEIQRA

SEQ ID NO: 72 - protein, 6 aa, Artificial Sequence
  CDR3 VH 1C1
EYDPLF

SEQ ID NO: 73 - protein, 8 aa, Artificial Sequence
  CDR3 VL 1C1
SSPSPSAV

SEQ ID NO: 74 - protein, 8 aa, Artificial Sequence
  CDR3 VL 3B6
SSMMKTPV

SEQ ID NO: 75 - protein, 6 aa, Artificial Sequence
  CDR3 VH 2B10
KRFGLF

SEQ ID NO: 76 - protein, 8 aa, Artificial Sequence
  CDR3 VL 2B10
SLPLNSTV

SEQ ID NO: 77 - protein, 6 aa, Artificial Sequence
  CDR3 VH 2A3
KRWGRF

SEQ ID NO: 78 - protein, 7 aa, Artificial Sequence
  CDR3 VL 2A3
SEPYLTV

SEQ ID NO: 79 - protein, 8 aa, Artificial Sequence
  CDR3 VH 1G5
KSKGLPYF

SEQ ID NO: 80 - protein, 8 aa, Artificial Sequence
  CDR3 VL 1G5
SHSLNPPV

SEQ ID NO: 81 - protein, 8 aa, Artificial Sequence
  CDR3 VH 3D9
KSKGVPYF

SEQ ID NO: 82 - protein, 8 aa, Artificial Sequence
  CDR3 VL 3D9
SQHRAQPV

SEQ ID NO: 83 - protein, 8 aa, Artificial Sequence
  CDR3 VH 2H10
KSQGLPYF

SEQ ID NO: 84 - protein, 8 aa, Artificial Sequence
  CDR3 VL 2H10
SADQAPVV

SEQ ID NO: 85 - protein, 8 aa, Artificial Sequence
  CDR3 VL 3C10
SAATGGPV

SEQ ID NO: 86 - protein, 6 aa, Artificial Sequence
  CDR3 VH 1E6
KVGKNF

SEQ ID NO: 87 - protein, 8 aa, Artificial Sequence
  CDR3 VL 1E6
SWDKVKPV

SEQ ID NO: 88 - protein, 6 aa, Artificial Sequence
  CDR3 VH 2G5
KVGRNF

SEQ ID NO: 89 - protein, 8 aa, Artificial Sequence
  CDR3 VL 2G5
SVSNRTPV

SEQ ID NO: 90 - protein, 6 aa, Artificial Sequence
  CDR3 VH 1C5
KGRFVF

SEQ ID NO: 91 - protein, 8 aa, Artificial Sequence
  CDR3 VL 1C5
SVWPWPRV

SEQ ID NO: 92 - protein, 7 aa, Artificial Sequence
  CDR3 VH 1C6
KRGPKSF

SEQ ID NO: 93 - protein, 8 aa, Artificial Sequence
  CDR3 VL 1C6
SMASSRPV

SEQ ID NO: 94 - protein, 7 aa, Artificial Sequence
  CDR3 VH 1C3
KVFAHGF

SEQ ID NO: 95 - protein, 8 aa, Artificial Sequence
  CDR3 VL 1C3
SLPPLHPV

SEQ ID NO: 96 - protein, 327 aa, Mus musculus
MGLPRRLLLLLLLATTCVPASQGLQCMQCESNQSCLVEECALGQDLCRTTVLREWQDDRELEVVTRGCAHSEKTNRTMSYRMGSMIISLTETVCATNLCNRPRPGARGRAFPQGRYLECASCTSLDQSCERGREQSLQCRYPTEHCIEVVTLQSTERSLKDQDYTRGCGSLPGCPGTAGFHSNQTFHFLKCCNYTHCNGGPVLDLQSFPPNGFQCYSCEGNNTLGCSSEEASLINCRGPMNQCLVATGLDVLGNRSYTVRGCATASWCQGSHVADSFPTHLNVSVSCCHGSGCNSPTGGAPRPGPAQLSLIASLLLTLGLWGVLLWT

(19) United States
(12) Patent Application Publication
(10) Pub. No.: US 2014/0161803 A1
(43) Pub. Date: Jun. 12, 2014

(54) CONSTITUTIVELY ACTIVE UPAR VARIANTS AND THEIR USE FOR THE GENERATION AND ISOLATION OF INHIBITORY ANTIBODIES

(71) Applicants: Nicolai Sidenius, Milano (MI) (IT); Sonu Gandhi, Milano (MI) (IT)

(72) Inventors: Nicolai Sidenius, Milano (MI) (IT); Sonu Gandhi, Milano (MI) (IT)

(73) Assignee: IFOM Fondazione Istituo FIRC di Oncologia Molecolare, Milano (MI) (IT)

(21) Appl. No.: 14/233,844

(22) PCT Filed: Aug. 2, 2012
(86) PCT No.: PCT/EP2012/065198

§ 371 Date: Feb. 4, 2014

Related U.S. Application Data
(60) Provisional application No. 61/515,506, filed on Aug. 5, 2011.

(51) Int. Cl. C07K 16/28; C07K 14/705

(52) U.S. Cl. CPC C07K 16/2896; C07K 14/70596
USPC 424/133.1; 530/387.3; 530/395; 424/139.1; 530/387.9; 506/9

(57) ABSTRACT

The invention relates to variants of the urokinase plasminogen activator receptor (uPAR) that display remarkably increased vitronectin (VN) binding activity, possibly caused by a more efficient exposure of the VN binding site. The present invention also refers to antibodies raised against said uPAR variants, able to bind to the VN binding site of uPAR and then acting as inhibitors of uPAR functions, acting as functional antagonists of VN activated-uPAR functions. In the present invention such antibodies are monoclonal, polyclonal, synthetic or recombinant derivatives thereof, as synthetic antibodies (scFv) from phage-display libraries. Antibodies of the invention act as competitive antagonists.

### BACKGROUND OF THE INVENTION

The urokinase plasminogen activator receptor (uPAR, also named CD87) is a membrane glycoprotein anchored to the plasma membrane by a glycosylphosphatidylinositol (GPI) anchor. The urokinase plasminogen activator (uPA) and its receptor (uPAR) play important roles in physiological processes such as wound healing, inflammation, and stem cell mobilization, as well as in severe pathological conditions such as HIV-1 infection, tumor invasion, and metastasis. The urokinase-type plasminogen activator receptor (uPAR) is a plasma membrane receptor overexpressed during inflammation and almost in all human cancers. The important role of uPAR in tumor cell adhesion, migration, invasion, and proliferation makes this receptor an attractive drug target in cancer treatment. Several therapeutic strategies inhibiting the uPA system have been or are currently being developed for suppression of tumor growth. Besides uPAR's well-established role in the regulation of pericellular proteolysis, it also modulates cell adhesion, migration, and proliferation through interactions with proteins present in the extracellular matrix, including vitronectin (VN).

Although the importance of the interaction with VN is well documented to be crucial for the signaling activity of uPAR (Madsen et al, 2007; Smith et al, 2008), the importance of this interaction in vivo has never been addressed.

A direct VN interaction is both necessary and sufficient to initiate uPAR-induced changes in cell morphology, migration, and signaling independently of direct lateral protein-protein interactions. The single interaction between uPAR and VN may be responsible for many of the proteolysis-independent biological effects initiated by uPAR. Development of inhibitors of the uPAR/vitronectin interaction is another attractive target and may possibly start from the uPAR-binding somatomedin B domain of vitronectin, which is a natural and potent uPAR/vitronectin interaction antagonist.

Several international applications disclose peptides ligand of urokinase receptor, such as WO01/17544.

WO97/35969 discloses peptides that are capable of binding to uPAR and inhibiting the binding of an integrin and vitronectin. The document does not refer to uPA binding. The binding site of the peptides in uPAR was not determined and no data on the function blocking activity of the peptides are presented in the document.

WO2008/073312 relates to urokinase-type plasminogen activator receptor epitope and monoclonal antibodies derived therefrom. The document discloses antibodies, and antigen-binding fragments thereof, specific for urokinase-type plasminogen activator receptor (uPAR) and their use for the treatment or prevention of cancer. In particular, the disclosed antibodies are specific for a particular epitope on uPAR. The antibodies described in WO2008/073312 recognize epitopes non-overlapping with those described in the present invention.

Rabbani S A, et al (Neoplasia (2010) 12, 778-788) examined the effects of administration of a monoclonal anti-uPAR antibody (ATN-658) on prostate cancer progression in vitro and in vivo. ATN-658, a mouse IgG1, is able to bind to D2D3 of uPAR with high affinity (K_{d}˜1 nM), does not inhibit the binding of uPA to uPAR, and is able to bind to uPAR even when uPA was also bound. The antibody used in this study (ATN-658) is that described in WO2008/073312. The epitope recognized by the ATN-658 antibody does not overlap with those described in the present invention. The ATN-658 antibody is not a competitive antagonist of the uPAR/vitronectin interaction as it does not bind to the vitronectin binding-site in uPAR. The ATN-658 antibody binds to an epitope in uPAR similar or identical to another well-characterized monoclonal antibody R2 (Sidenius et al. JBC (2002) 277 27982-90). ATN-658 binds to intact uPAR and the truncated D2D3 receptor equally well. Thus, the antibody therein described does not bind preferentially to intact uPAR.

WO2005/116077 identifies antibodies or other ligands specific for the binary uPA-uPAR complexes, for ternary complexes comprising uPA-uPAR and for complexes of uPAR and proteins other than uPA such as integrins. The antibodies inhibit the interaction of uPA and uPAR with additional molecules with which the complex interact. Such antibodies or other ligands are used in diagnostic and therapeutic methods, particularly against cancer. The document refers to ligands that do not inhibit vitronectin binding but the assembly of vitronectin components; moreover, they recognize epitopes non-overlapping with those herein described.

WO2006/094828 discloses antibodies that preferentially recognize truncated and soluble forms of uPAR receptor (D2 D3). The antibodies therein described do not bind preferentially to intact uPAR.

CN101050237 discloses a compound that can block interactions between uPA and uPAR, and its application. The compound comprises ATF of uPA, ATF fragment, uPAR fragment, anti-ATF antibody, and anti-uPAR antibody. The compound can block the interactions between uPA and uPAR, and can be used to prepare medicine for preventing and treating atherosclerosis.

Tressler R J et al., (APMIS. 1999 January; 107(1):168-73) discloses urokinase receptor antagonists based on the growth factor domains of both human and murine urokinase. Such antagonists show sub-nanomolar affinities for their homologous receptors. Further modification of these molecules by preparing fusions with the constant region of human IgG has led to molecules with high affinities and long in vivo half-lives. Smaller peptide inhibitors have been obtained by a combination of bacteriophage display and peptide analogue synthesis. All of these molecules inhibit the binding of the growth factor domain of uPA to the uPA receptor and enhance binding of the uPA receptor to vitronectin.

Gardsvoll H, et al (J Biol Chem, 2011 Sep. 23; 286(38):33544-56) proposes a model of cooperation between uPA and vitronectin to potentiate uPAR-dependent induction of lamellipodia on vitronectin matrices; this will have implications for drug development targeting uPAR function, i.e. epitope-mapped monoclonal antibodies. None of the antibodies investigated in this study block vitronectin binding to uPAR in the presence of uPA.

There is thus the need of antibodies which bind preferentially to intact uPAR and which are potent inhibitors of uPAR-functions.

### DESCRIPTION OF THE INVENTION

The present invention concerns unique variants of the urokinase plasminogen activator receptor (uPAR) that display remarkably increased vitronectin (VN) binding activity (>10.000-fold increased apparent Kd), possibly caused by a more efficient exposure of the VN binding site.

Authors showed that monoclonal antibodies raised against said uPAR-variants are potent inhibitors of uPAR-functions. Mapping of the antibody binding epitopes shows that these antibodies bind to the VN binding site of uPAR classifying them as competitive antagonists.

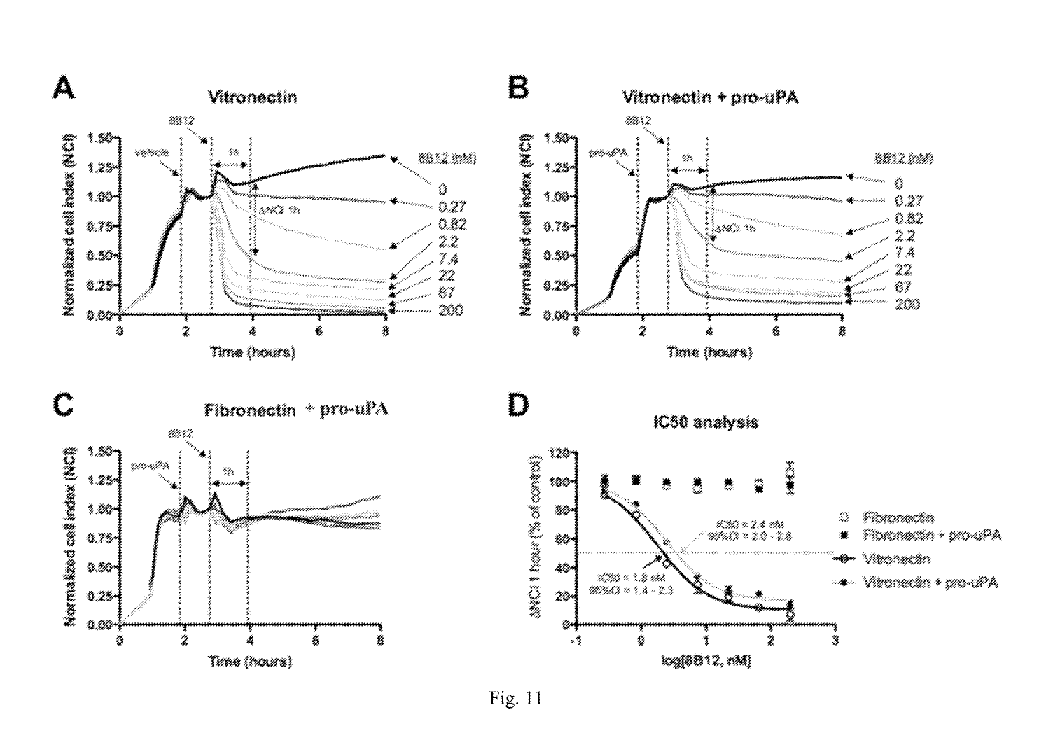

The authors also showed that the above uPAR-variants are used to isolate synthetic antibodies (scFv) from phage-display libraries which are functional inhibitors of uPAR. The inhibited functions are: cell adhesion (FIG. 11) and consequently cell migration and cell proliferation (which are downstream events in respect of cell adhesion). These functions are VN-dependent.

### DETAILED DESCRIPTION OF THE INVENTION

Object of the present invention is an urokinase plasminogen activator receptor (uPAR) variant molecule having an increased VN-binding activity with respect to the wild type molecule.

The uPAR variant molecule according to the invention preferably comprises a wild type uPAR amino acid sequence linked to:
a) a growth factor-like domain (GFD) sequence of uPA at the N-terminal of the wild type uPAR sequence, and/or
b) a chain of the Fc region of an antibody molecule at the C-terminal of the wild type uPAR sequence,
   wherein if said chain of the Fc region is present, the uPAR variant molecule is a dimer.

For “wild type uPAR amino acid sequence” it is intended the sequence of the full wild type protein or fragments thereof maintaining a VN-binding activity.

In a preferred embodiment the wild type uPAR sequence comprises a sequence consisting essentially of the aa. 32-92 of mature huPAR of SEQ ID NO: 1 or a sequence consisting essentially of the aa. 32-93 of mature muPAR of SEQ ID NO: 2 or a polypeptide encoded by the correspondent regions from an uPAR orthologous gene, or functional mutants or derivatives or analogues thereof.

More preferably the wild type uPAR sequence comprises a sequence consisting essentially of the aa. 3-271 of mature huPAR of Seq ID NO: 1 or a sequence consisting essentially of the aa. 3-270 of mature muPAR of Seq ID NO: 2 or a polypeptide encoded by the correspondent regions from an uPAR orthologous gene, or functional mutants or derivatives or analogues thereof.

Even more preferably, the wild type uPAR sequence comprises a sequence consisting essentially of the aa. 1-277 of mature huPAR of Seq ID NO: 1 or a sequence consisting of essentially the aa. 1-273 of mature muPAR of Seq ID NO: 2 or a polypeptide encoded by the correspondent regions from an uPAR orthologous gene, or functional mutants or derivatives or analogues thereof.

In another preferred embodiment of the invention, the wild type uPAR sequence comprises a sequence consisting essentially of Seq ID NO: 1 or Seq ID NO: 2 or a polypeptide encoded by the correspondent region from a uPAR orthologous gene, or functional mutants or derivatives or analogues thereof.

In the present invention, the GFD sequence of uPA preferably comprises a sequence consisting essentially of the aa. 11-42 of the GFD of human uPA of SEQ ID NO: 3 or a sequence consisting essentially of the aa. 12-43 of the GFD of mouse uPA of SEQ ID NO: 4 or a polypeptide encoded by the correspondent region from a GDF orthologous gene, or functional mutants or derivatives or analogues thereof.

In the uPAR variant molecule according to the invention the GFD sequence of uPA preferably consists essentially of the GFD sequence of human uPA of SEQ ID NO: 3 or of the GFD sequence of mouse uPA of SEQ ID NO: 4 or a polypeptide encoded by the correspondent region from a GFD orthologous gene, or functional mutants or derivatives or analogues thereof.

In the present invention, the chain of the Fc region is preferably of human origin and comprises a sequence consisting essentially of SEQ ID NO: 5 or the chain of the Fc region is preferably of mouse origin and comprises a sequence consisting essentially of SEQ ID NO: 6 or a polypeptide encoded by the correspondent region from a chain of the Fc region orthologous gene, or functional mutants or derivatives or analogues thereof.

In the uPAR variant molecule according to the invention, the human chain of the Fc region preferably consists essentially of SEQ ID NO: 5, or the mouse Fc region preferably consists essentially of SEQ ID NO: 6 or a polypeptide encoded by the correspondent region from a human chain of the Fc region orthologous gene, or functional mutants or derivatives or analogues thereof.

In a preferred embodiment, the uPAR variant molecule of the invention further comprises:

a) a first linker region between the GFD sequence of uPA and the N-terminal of the wild type uPAR sequence, and/or
b) a second linker region between the chain of the Fc region of an antibody molecule and the C-terminal of the wild type uPAR sequence.

Preferably, said first linker region consists essentially of the sequence of SEQ ID NO: 7 or SEQ ID NO: 8.

The second linker region preferably consists essentially of the sequence of SEQ ID NO: 9, SEQ ID NO: 10 or SEQ ID NO: 11.

In a preferred embodiment, the uPAR variant molecule comprises a sequence having essentially the sequence of SEQ ID NOs: 12, 13, 14, 15, 16 or 17.

Another object of the invention is the use of the uPAR variant molecule according to the invention as antigen for obtaining a specific antibody molecule having an antagonist activity of uPAR functions or for selecting a recombinant or synthetic antigen-binding fragments of said antibody.

A further object of the invention is an antibody, recombinant or synthetic antigen-binding fragments thereof able to bind the urokinase plasminogen activator receptor (uPAR) variants as above described. Said antibody, recombinant or synthetic antigen-binding fragments thereof preferably have an antagonist activity of uPAR functions.

The antibodies are useful for therapeutic applications in humans. Typically, the antibodies are fully human or chimeric or humanized to minimize the risk for immune responses against the antibodies when administered to a patient. As described herein, other antigen-binding molecules such as, e.g., antigen- binding antibody fragments, antibody derivatives, and multi-specific molecules, can be designed or derived from such antibodies.

Antibody-binding fragments of such antibodies, as well as molecules comprising such antigen-binding fragments, including engineered antibody fragments, antibody derivatives, bispecific antibodies and other multispecific molecules, are also object of the invention.

In a preferred embodiment, the antibody, recombinant or synthetic antigen-binding fragments thereof according to the invention, are able to bind to an epitope of uPAR molecule, said epitope comprising at least one of R89, R91 and Y92 amino acid residues.

Preferably, the antibody, recombinant or synthetic antigen-binding fragments thereof according to the invention comprise at least one heavy chain complementary determining region (CDRH3) amino acid sequence having at least 80% identity to an amino acid sequence selected from the group consisting of: aa. 90-102 of SEQ ID NO: 18, 19, 20, 21 or 25, aa. 90-101 of SEQ ID NO: 24, and SEQ ID NO: 22 or 23, and/or at least one heavy chain complementary determining region (CDRH2) amino acid sequence having at least 80% identity to an amino acid sequence selected from the group consisting of: aa. 41-57 of SEQ ID NO: 18, 19, 20, 21, 24 or 25, and/or at least one heavy chain complementary determining region (CDRH1) amino acid sequence having at least 80% identity to an amino acid sequence selected from the group consisting of: aa. 22-26 of SEQ ID NO: 18, 19, 20, 21 or 24; aa. 17-26 of SEQ ID NO: 25.

Preferably, the antibody, recombinant or synthetic antigen-binding fragments thereof according the invention, comprise at least one light chain complementary determining region (CDRL3) amino acid sequence having at least 80% identity to an amino acid sequence selected from the group consisting of: aa. 80-87 of SEQ ID NO: 65, 66, 67, 68 or 69, and SEQ ID NO: 74 or 85 and/or at least one light chain complementary determining region (CDRL2) amino acid sequence having at least 80% identity to an amino acid sequence selected from the group consisting of: aa. 41-47 of SEQ ID NOs: 65, 66, 67, 68 and 69 and/or at least one light chain complementary determining region (CDRL1) amino acid sequence having at least 80% identity to an amino acid sequence selected from the group consisting of: aa. 15-23 of SEQ ID NOs: 65, 66, 67, 68 and 69.

In a preferred embodiment, the antibody, recombinant or synthetic antigen-binding fragments thereof as described above comprises a CDRH1 amino acid sequence having at least 80% identity to aa. 22-26 of SEQ ID NO: 18, 19, 20, 21 or 24, a CDRH2 amino acid sequence having at least 80% identity to aa. 41-57 of SEQ ID NO: 18, 19, 20, 21 or 24, respectively and a CDRH3 amino acid sequence having at least 80% identity to aa. 90-102 of SEQ ID NO: 18, 19, 20 or 21, or aa. 90-101 of SEQ ID NO: 24 respectively.

In another preferred embodiment, the antibody, recombinant or synthetic antigen-binding fragments thereof as described above comprises a CDRH1 amino acid sequence having at least 80% identity to aa. 17-26 of SEQ ID NO: 25, a CDRH2 amino acid sequence having at least 80% identity to aa. 41-57 of SEQ ID NO: 25, and a CDRH3 amino acid sequence having at least 80% identity to aa. 90-102 of SEQ ID NO: 25.

In a still preferred embodiment, the antibody, recombinant or synthetic antigen-binding fragments thereof of the invention further comprises a CDRL1 amino acid sequence having at least 80% identity to aa. 15-23 of SEQ. ID NO: 65, 66, 67, 68 or 69, a CDRL2 amino acid sequence having at least 80% identity to aa. 41-47 of SEQ ID NO: 65, 66, 67, 68 or 69 respectively and a CDRL3 amino acid sequence having at least 80% identity to aa. 80-87 of SEQ ID NO: 65, 66, 67, 68 or 69, respectively.

In a still preferred embodiment, the antibody, recombinant or synthetic antigen-binding fragments thereof of the invention further comprises a CDRH1 amino acid sequence having at least 80% identity to aa. 22-26 of SEQ ID No. 18, 19, 20, 21 or 24, a CDRH2 amino acid sequence having at least 80% identity to aa. 41-57 of SEQ ID No. 18, 19, 20, 21 or 24, respectively, CDRH3 amino acid sequence having at least 80% identity to aa. 90-102 of SEQ ID NO: 18, 19, 20 or 21, or aa. 90-101 of SEQ ID NO: 24 respectively, a CDRL1 amino acid sequence having at least 80% identity to aa. 15-23 of SEQ ID NO: 66, 65, 68, 67 or 69 respectively, a CDRL2 amino acid sequence having at least 80% identity to aa. 41-47 of SEQ ID NO: 66, 65, 68, 67 or 69 respectively and a CDRL3 amino acid sequence having at least 80% identity to aa. 80-87 of SEQ ID NO: 66, 65, 68, 67 or 69 respectively.

In another aspect, the antibody, recombinant or synthetic antigen-binding fragments thereof according the invention comprise a heavy chain variable region comprising an amino acid sequence having at least 80% identity to an amino acid sequence selected from the group consisting of: SEQ. ID NOs: 18, 19, 20, 21, 24 and 25 and/or a light chain variable region comprising an amino acid sequence having at least 80% identity to an amino acid sequence selected from the group consisting of: SEQ ID NOs: 65, 66, 67, 68 or 69.

In a preferred embodiment, the antibody, recombinant or synthetic antigen-binding fragments thereof according the invention comprise a heavy chain variable region comprising an amino acid sequence having at least 80% identity to an amino acid sequence selected from the group consisting of: SEQ ID NOs: 18, 19, 20, 21 and 24 and a light chain variable region comprising an amino acid sequence having at least 80% identity to an amino acid sequence selected from the group consisting of: SEQ ID NO: 66, 65, 68, 67 and 69 respectively.

In the present invention “at least 80% identity” means that the identity may be at least 80% or at least 85% or 90% or 95% or 100% sequence identity to referred sequences.

Preferably, the antibody, recombinant or synthetic antigen-binding fragments thereof as described above is a monoclonal antibody or a chimeric or a humanized, or a deimmunized or a fully human antibody.

Another object of the invention is the antibody, recombinant or synthetic antigen-binding fragments thereof as above described for use as a medicament, in particular for use in the treatment of cancer, preferably in the treatment of prostate cancer.

It is a further object of the invention a nucleic acid molecule encoding the antibody, recombinant or synthetic antigen-binding fragments thereof as defined above or hybridizing with the above nucleic acid, or consisting of a degenerated sequence thereof. It is a further object of the invention an expression vector encoding the antibody, recombinant or synthetic antigen-binding fragments thereof of the invention. It is a further object of the invention a host cell comprising the nucleic acid as described above. Preferably, the host cell produces the antibody, recombinant or synthetic antigen-binding fragments thereof of the invention.

It is a further object of the invention a method of producing the antibody, recombinant or synthetic antigen-binding fragments thereof of the invention comprising culturing the cell that produces the antibody as described above and recovering the antibody from the cell culture.

In the present invention mutants of the disclosed CDRs may be generated by mutating one or more amino acids in the sequence of the CDRs. It is known that a single amino acid substitution appropriately positioned in a CDR can be sufficient to improve the affinity. Researchers have used site directed mutagenesis to increase affinity of some immunoglobulin products by about 10 fold. This method of increasing or decreasing (i.e modulating) affinity of antibodies by mutating CDRs is common knowledge (see, e.g., Paul, W. E., 1993). Thus, the substitution, deletion, or addition of amino acids to the CDRs of the invention to increase or decrease (i.e. modulate) binding affinity or specificity is also within the scope of this invention.

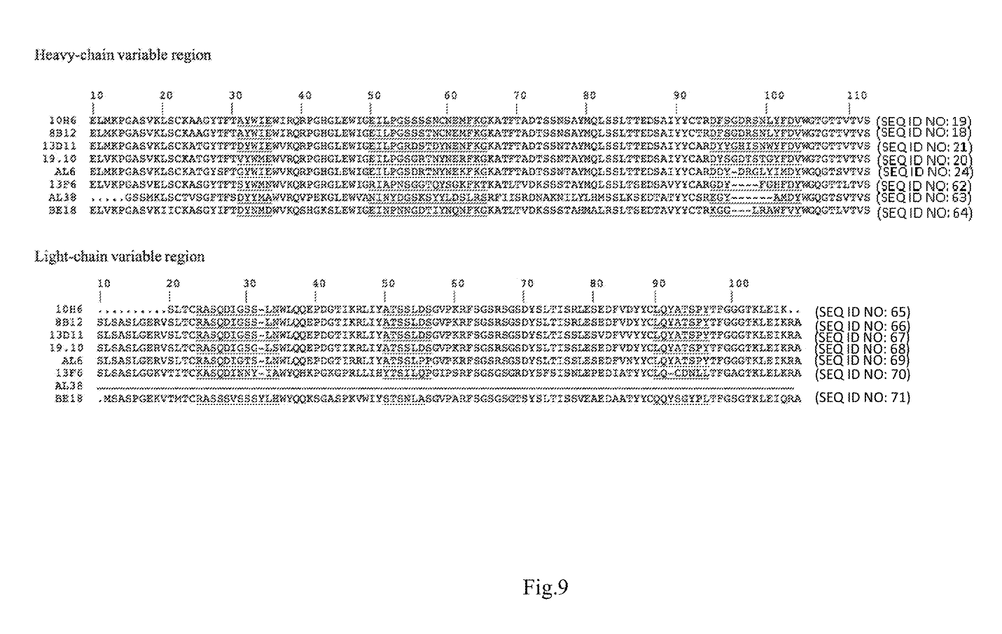

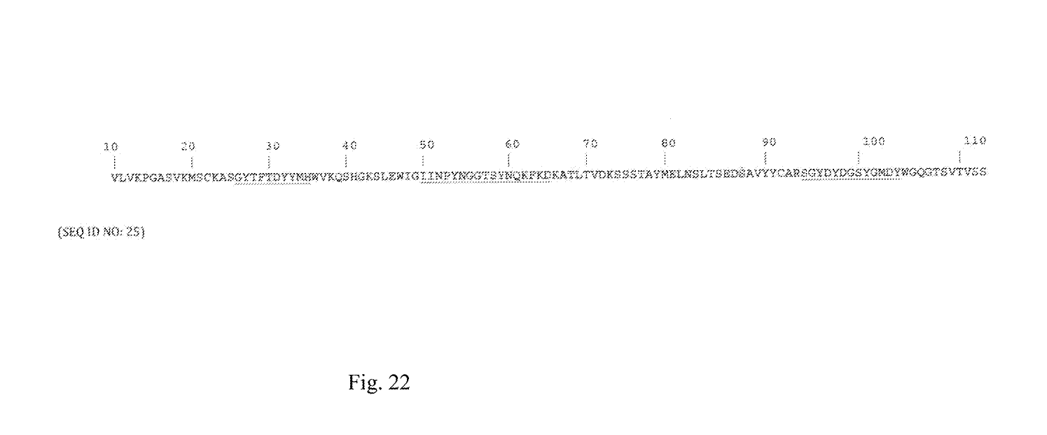

For sake of brevity, the preferred antibodies according to the present invention shall be identified with the name 10H6 (comprising SEQ ID NO: 19 and SEQ ID NO: 65), 8B12 (comprising SEQ ID NO: 18 and SEQ ID NO: 66), 13D11 (comprising SEQ ID NO: 21 and SEQ ID NO: 67), 19.10 (comprising SEQ ID NO: 20 and SEQ ID NO: 68), AL6 (comprising SEQ ID NO: 24 and SEQ ID NO: 69) (as indicated in FIG. 9), OMD4 (comprising SEQ ID NO: 25) (as indicated in FIG. 22). While the present invention focuses on such antibodies, as an exemplification of the present invention, one of ordinary skill in the art will appreciate that, once given the present disclosure, other similar antibodies, and antibody fragments thereof, as well as antibody fragments of these similar antibodies may be produced and used within the scope of the present invention. Such similar antibodies may be produced by a reasonable amount of experimentation by those skilled in the art.

Still preferably, the antibody is a scFv, Fv fragment, a Fab fragment, a F(ab)2 fragment, a multimeric antibody, a peptide or a proteolytic fragment containing the epitope binding region. Preferably the scFv fragment comprises

a) SEQ ID NOs: 22 and/or 74 (herein identified with the name 3B6, as indicated in table 3) or
b) SEQ ID NOs: 23 and/or 85 (herein identified with the name 3C10, as indicated in table 3). Kits or other articles that comprise the antibodies of the invention are also part of the invention.

A further object of the invention is a pharmaceutical composition comprising at least one antibody, recombinant or synthetic antigen-binding fragments thereof as above described and appropriated diluents and/or excipients. The composition comprises an effective amount of the antibody, recombinant or synthetic antigen-binding fragments thereof. Pharmaceutical compositions are conventional in this field and can be made by the person skilled in the art just based on the common general knowledge. Pharmaceutical compositions comprising the antibody and/or a fragment and/or a recombinant derivative and/or a conjugate thereof in admixture with at least one pharmaceutically acceptable excipient and/or vehicle are included in the scope of the present invention.

It is also an object of the invention a method of treating and/or preventing cancer in a subject, the method comprising administering to a subject in need thereof a therapeutically effective amount of the antibody, recombinant or synthetic antigen-binding fragments thereof as described above. It is an object of the invention a method of reducing and/or inhibiting uPAR comprising administering an effective amount of the antibody, recombinant or synthetic antigen-binding fragments thereof as described above.

The invention provides formulations comprising a therapeutically effective amount of an antibody as disclosed herein, a buffer maintaining the pH in the range from about 4.5 to about 6.5, and, optionally, a surfactant. The formulations are typically for an antibody as disclosed herein, recombinant or synthetic antigen-binding fragments thereof of the invention as active principle concentration from about 0.1 mg/ml to about 100 mg/ml. In certain embodiments, the antibody, recombinant or synthetic antigen-binding fragments thereof concentration is from about 0.1 mg/ml to 1 mg/ml; preferably from 1 mg/ml to 10 mg/ml, preferably from 10 to 100 mg/ml. For the purposes herein, a “pharmaceutical composition” is one that is adapted and suitable for administration to a mammal, especially a human Thus, the composition can be used to treat a disease or disorder in the mammal Moreover, the antibody in the composition has been subjected to one or more purification or isolation steps, such that contaminant(s) that might interfere with its therapeutic use have been separated therefrom.

Generally, the pharmaceutical composition comprises the therapeutic protein and a pharmaceutically acceptable carrier or diluent. The composition is usually sterile and may be lyophilized. Pharmaceutical preparations are described in more detail below. Therapeutic formulations of the antibody/antibodies can be prepared by mixing the antibody having the desired degree of purity with optional physiologically acceptable carriers, excipients or stabilizers (Remington's Pharmaceutical Sciences 16th edition, Osol, A. Ed., 1980), in the form of lyophilized formulations or aqueous solutions. Acceptable carriers, excipients, or stabilizers are nontoxic to recipients at the dosages and concentrations employed, and may include buffers, antioxidants, preservatives, peptides, proteins, hydrophilic polymers, chelating agents such as EDTA, sugars, salt-forming counter-ions such as sodium; metal complexes (e.g., Zn-protein complexes); and/or non-ionic surfactants such as TWEEN®, PLURONICS® or polyethylene glycol (PEG). The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed., 1980). The formulations to be used for in vivo administration must be sterile. This is readily accomplished by filtration through sterile filtration membranes.

In another embodiment, for the prevention or treatment of disease, the appropriate dosage of the antibody/antibodies of the present invention, will depend on the type of disease to be treated, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg of antibody or fragment thereof is an initial candidate dosage for administration to the patient, whether, for example, by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays. The antibody composition should be formulated, dosed, and administered in a fashion consistent with good medical practice. The antibodies/derivatives of the present invention can be administered by any appropriate route. This includes (but is not limited to) intraperitoneal, intramuscular, intravenous, subcutaneous, intraarticular, intratracheal, oral, enteral, parenteral, intranasal or dermal administration. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to prevent, ameliorate, or treat a disease or disorder. The antibody need not be, but is optionally formulated with one or more agents currently used to prevent or treat the disorder in question. The effective amount of such other agents depends on the amount of antibody present in the formulation, the type of disorder or treatment, and other factors discussed above.

In the present invention an antibody refers to:
a) a monoclonal, a polyclonal or a chimeric, or a humanized, or a deimmunized, or an affinity matured antibody, or a fully human antibody or a scFv;
b) a recombinant or synthetic antigen-binding fragments thereof, as well as molecules comprising such antigen-binding fragments, including engineered antibody fragments, antibody derivatives, bispecific antibodies and other multispecific molecules.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “chimeric” antibody refers to an antibody in which a portion of the heavy and/or light chain is derived from a particular source or species, while the remainder of the heavy and/or light chain is derived from a different source or species.

The term “Fc region” herein is preferably used to define a C-terminal region of an antibody, preferably an immunoglobulin, more preferably a human IgG, heavy chain that contains at least a portion of the constant region, more preferably it is used to define the human IgG hinge and constant region (hFc) or mouse IgG hinge and constant region (mFc). Similar sequences from other immunoglobulin types and/or species which form dimers or oligomers are included in the term. The term also includes native sequence Fc regions and variant Fc regions.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “deimmunized” antibody is an antibody with reduced immunogenicity based on disruption of HLA binding, an underlying requirement for T cell stimulation.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term “pharmaceutical formulation” refers to a preparation which is in such form as to permit the biological activity of an active ingredient contained therein to be effective, and which contains no additional components which are unacceptably toxic to a subject to which the formulation would be administered.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The antibody, recombinant or synthetic antigen-binding fragments thereof of the invention can be conjugated to a molecole, said molecule is preferably a therapeutic agent.

The invention will be now described by non-limiting examples referring to the following figures:

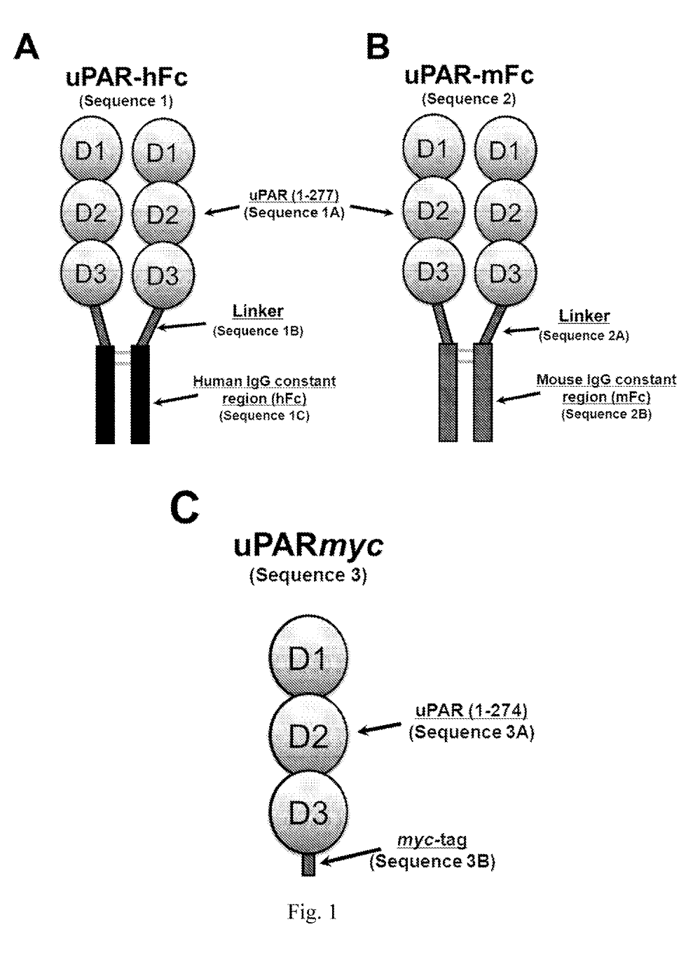

FIG. 1: Cartoon illustrating the domain structure of three uPAR variants, uPAR-hFc, uPAR-mFc and uPARmyc (as control), respectively. (A) Cartoon illustrating the structure of uPAR-hFc—a soluble dimeric form of uPAR with a human Fc tag. uPAR-hFc (Sequence 1 corresponding to SEQ ID NO:14) is composed of residues 1-277 (Sequence 1A, corresponding to aa. 1-277 of SEQ ID NO: 1, domains D1, D2 and D3) of human uPAR (full sequence in Sequence 4 corresponding to SEQ ID NO: 1), a linker region (Sequence 1B corresponding to SEQ ID NO: 9) and the hinge and constant regions (Fc) of a human IgG heavy chain (Sequence 1C corresponding to SEQ ID NO: 5). The presence of the hFc-tag results in the formation of homodimer where the two polypeptides are linked together by disulfide bonds.

(B) Cartoon illustrating the structure of uPAR-mFc—a soluble dimeric form of uPAR with a mouse Fc tag. uPAR-mFc (Sequence 2 corresponding to SEQ ID NO: 15) is composed of residues 1-277 of human uPAR (Sequence 1A, corresponding to aa. 1-277 of SEQ ID NO: 1), a linker region (Sequence 2A corresponding to SEQ ID NO: 10) and the hinge and constant regions (Fc) of a murine IgG heavy chain (Sequence 2B corresponding to SEQ ID NO: 6). As for uPAR-hFc, the presence of the mFc-tag results in the formation of homodimer where the two polypeptides are linked together by disulfide bonds. (C) Cartoon illustrating the structure of uPARmyc—a soluble monomeric form of uPAR with a C-terminal myc-tag. uPARmyc (Sequence 3 corresponding to SEQ ID NO: 26) is composed of residues 1-274 of human uPAR (Sequence 3A corresponding to aa. 1-274 of SEQ ID NO: 1) and a C-terminal myc-tag (Sequence 3B corresponding to SEQ ID NO: 27). As indicated, mature wild-type uPAR is composed of three homologous domains termed D1, D2 and D3.

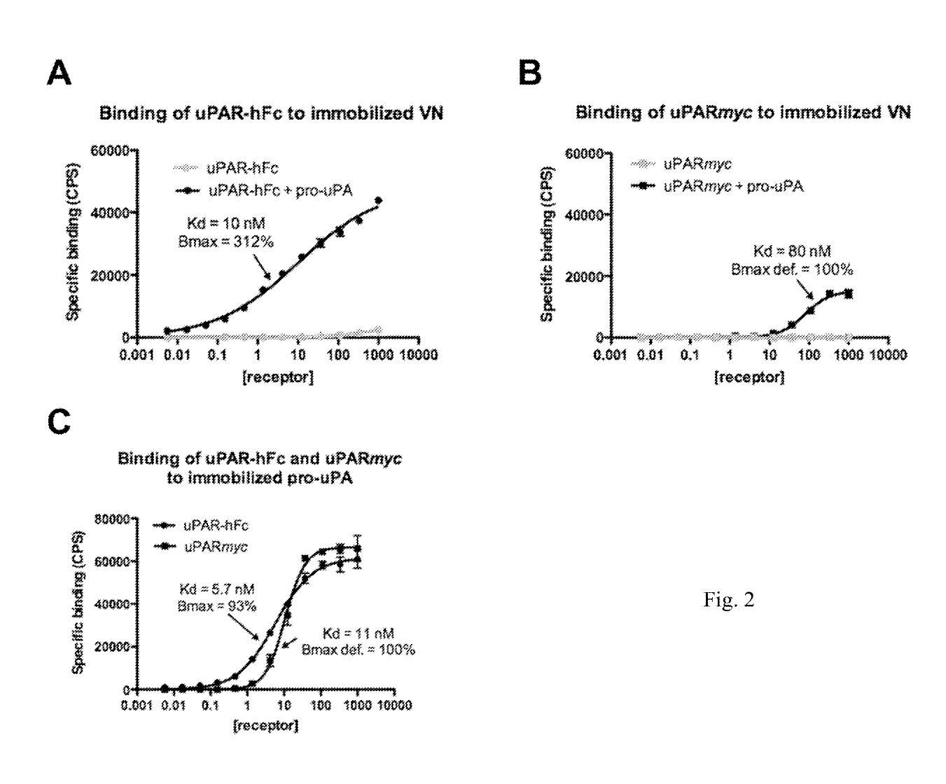

FIG. 2: Forced dimerization of uPAR using immunoglobulin heavy chain constant regions increases the binding affinity for VN, but not for uPA. (A) Binding of uPAR-hFc to immobilized VN. 96-well plates coated with VN were incubated with increasing concentrations of uPAR-hFc in the presence (black) or absence (grey) of excess pro-uPA for 2 hours at room temperature. After washing, bound receptor was detected by sequential incubations with a monoclonal uPAR antibody (13F6) and a Eu3+-labeled goat-anti mouse antibody. The bound material was detected by measuring time-resolved fluorescence intensity. Specific binding was calculated by subtracting the non-specific binding measured in uncoated wells incubated with identical samples. The data shown are means±SD from a representative experiment. The binding curve, equilibrium dissociation constant (Kd, in nanomolar units) and maximum binding capacity (Bmax, in CPS units (counts per second)) were calculated by non-linear regression (four-parameter fit) using the Prism 5.0 software suite. Note that uPAR-hFc has ˜10-fold higher affinity and ˜3-fold higher binding capacity than that of the monomeric uPARmyc shown in Panel B. (B) Binding of uPARmyc to immobilized VN. 96-well plates coated with VN were incubated with increasing concentrations of uPARmyc in the presence (black) or absence (grey) of excess pro-uPA for 2 hours at room temperature. Binding of uPARmyc was detected and analyzed exactly as described in panel A. (C) Binding of uPAR-hFc and uPARmyc to immobilized pro-uPA. 96-well plates coated with pro-uPA were incubated with increasing concentrations of uPAR-hFc (circles) and uPARmyc (squares) for 2 hours at room temperature. Binding of uPARmyc was detected and analyzed exactly as described in panel A. Note that uPAR-hFc and uPARmyc bind to immobilized pro-uPA with very similar Kd and Bmax.

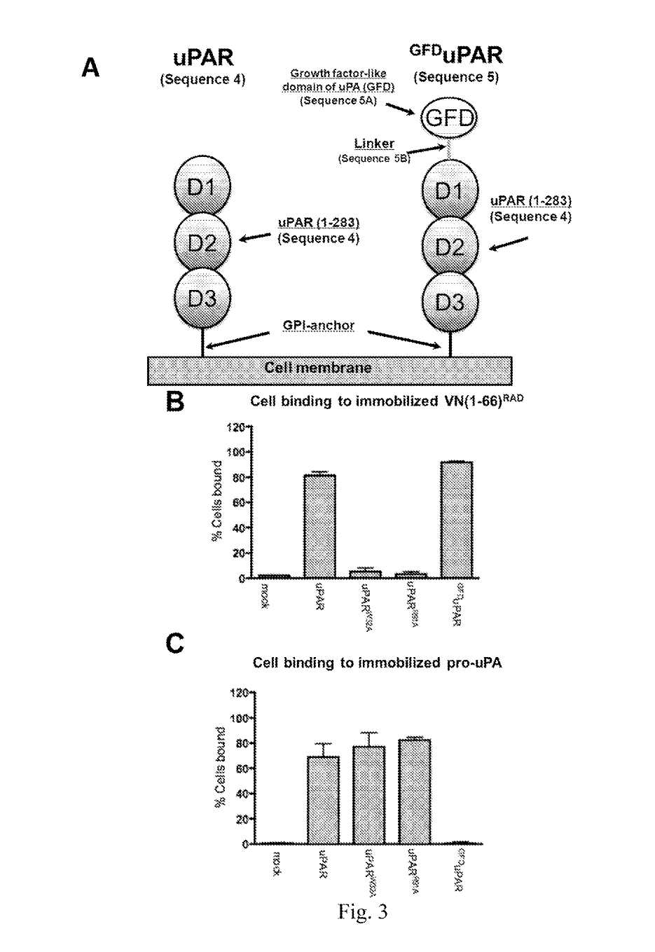

FIG. 3: uPAR variant made by a chimeric molecule between the growth factor-like domain of uPA and uPAR through its N-terminal binding increases VN-binding and reduces uPA-binding. (A) Cartoon illustrating the domain structure of wild-type human uPAR and the uPAR variant GFD-uPAR chimera. Mature wild-type uPAR (Sequence 4 (SEQ ID NO: 1)) is composed of 3 homologous protein domains (D1, D2 and D3) that is linked to outer leaflet of the cell membrane by glycosylphosphatidylinositol (GPI) lipid anchor located on the C-terminal of uPAR. The GFDuPAR-chimera (Sequence 5 (SEQ ID NO: 16)) has the same sequence as wild-type uPAR (Sequence 4 (SEQ ID NO: 1)) but contains in addition the receptor-binding growth factor-like domain of uPA, GFD (Sequence 5A (SEQ ID NO: 3)), engineered onto the N-terminal of uPAR (Sequence 4 (SEQ ID NO: 1)) using a short linker sequence (Sequence 5B (SEQ ID NO: 7)). (B) Expression of GFDuPAR in 293 cells promotes cell adhesion to vitronectin. 293 cells expressing either wild type uPAR (uPAR), uPAR mutants with deficient VN-binding (uPARW32A and uPARR91A, (Madsen et al., 2007)), the GFDuPAR chimera (GFDuPAR) or no uPAR (mock) were allowed to adhere for 1 hour at 37° C. to wells coated with a VN-fragment deficient in integrin binding (VN(1-66)RAD, (Madsen et al., 2007)). After washing, the adherent cells were fixed, stained with crystal violet and quantified by measuring the absorbance at 530 nm. The specific cell adhesion was calculated by subtracting non-specific binding (measured in uncoated wells) and is presented in % of adhesion to poly-L-lysine. The data represents the mean±SD of independent experiments (n=3). Note that uPAR and GFDuPAR both promote robust cell adhesion to VN while the W32A and R91A mutant receptors, as well as mock-transfected cells, fail to adhere. (C) The GFDuPAR-chimera is deficient in promoting cell adhesion to immobilized pro-uPA. 293 cells expressing the different uPAR variants were allowed to bind to wells coated with pro-uPA for 1 hour at 37° C. and cell adhesion quantified as described in Panel B. Note that expression of uPAR, uPARW32A and uPARR91A induces firm cell adhesion to pro-uPA while the GFDuPAR-chimera does not, thus demonstrating that this chimera is deficient in pro-uPA binding.

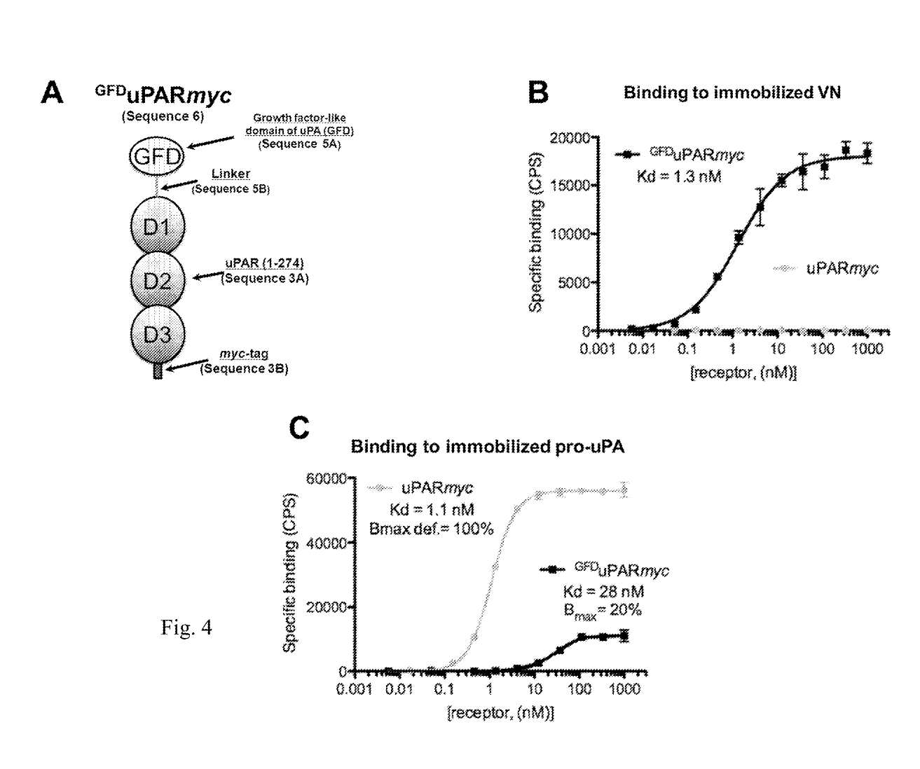

FIG. 4: Soluble GFDuPAR displays uPA-independent high-affinity binding to VN and reduced uPA binding. (A) Cartoon illustrating the domain organization of GFDuPARmyc. GFDuPARmyc (Sequence 6 (SEQ ID NO: 28)) is a secreted variant of GFDuPAR (FIG. 3A) containing a C-terminal myc-tag. The composition of the GFDuPARmyc-chimera (Sequence 6 (SEQ ID NO: 28)) is the receptor-binding growth factor-like domain of uPA, GFD (Sequence 5A (SEQ ID NO: 3)), a short linker (Sequence 5B (SEQ ID NO: 7)), uPAR residues 1-274 (Sequence 3A corresponding to aa. 1-274 of SEQ ID NO: 1) and a C-terminal myc-tag (Sequence 3B (SEQ ID NO: 27)). (B) Binding of GFDuPARmyc and uPARmyc to immobilized VN. 96-well plates coated with VN were incubated with increasing concentrations of GFDuPARmyc (black squares) and uPARmyc (grey circles) for 2 hours at room temperature. After washing, the bound receptor was detected by sequential incubations with a monoclonal uPAR antibody (13F6) and a Eu3+-labeled goat-anti mouse antibody. The bound material was detected by measuring time-resolved fluorescence intensity. Specific binding was calculated by subtracting the non-specific binding measured in uncoated wells incubated with identical samples. The shown data are means±SD from a representative experiment. The binding curve and dissociation constant (Kd) were calculated by non-linear regression (four-parameter fit) using the Prism 5.0 software suite. Note that GFDuPARmyc, but not uPARmyc, binds VN with high affinity. (C) Binding of GFDuPARmyc and uPARmyc to immobilized pro-uPA. 96-well plates coated with pro-uPA were incubated with increasing concentrations of GFDuPARmyc (black squares) and uPARmyc (grey circles) for 2 hours at room temperature and bound receptor detected as described in panel B. The binding curves, Kd and Bmax were calculated by non-linear regression as above. Note that GFDuPARmyc binds uPA with ˜30-fold reduced affinity and ˜5-fold decreased binding capacity, as compared to uPARmyc.

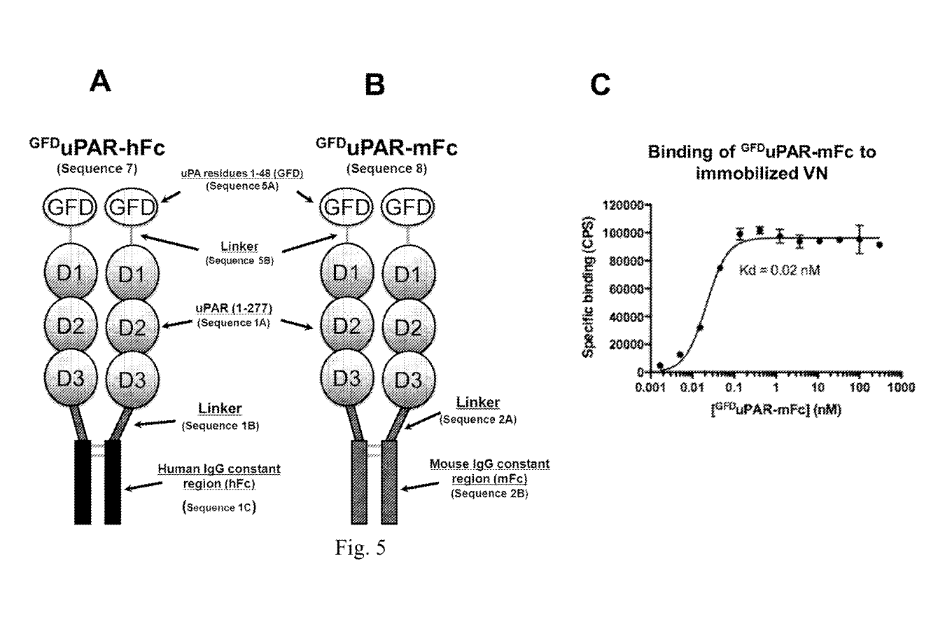

FIG. 5: Other uPAR variants: forced dimerization and the addition of GFD domain on the N-terminal of uPAR synergize to increase the VN-binding activity of the receptor (A) Cartoon illustrating the domain structure of GFDuPAR-hFc. GFDuPAR-hFc (Sequence 7 (SEQ ID NO: 12)) combines forced dimerization by addition of a C-terminal human Fc-tag as shown in FIG. 1A with the appending of the GFD-domain on the N-terminal as shown in FIG. 3A. (B) Cartoon illustrating the domain structure of GFDuPAR-mFc. GFDuPAR-mFc (Sequence 8 (SEQ ID NO: 13)) is identical to GFDuPAR-hFc with the exception that the Fc-region originates from a mouse immunoglobulin (see FIG. 1B). (C) GFDuPAR-mFc binds with extremely high affinity to immobilized VN. 96-well plates coated with VN were incubated with increasing concentrations of GFDuPAR-mFc for 2 hours at room temperature. After washing, the bound receptor was detected by sequential incubations with a biotinylated antibody specific for the constant region of mouse IgG and Eu3+-labeled streptavidin. Bound material was quantified by measuring time-resolved fluorescence. Specific binding was calculated by subtracting non-specific binding measured in uncoated wells incubated with the same samples. The data represents means±SD and are from a representative experiment. The binding curve and Kd were calculated by non-linear regression.

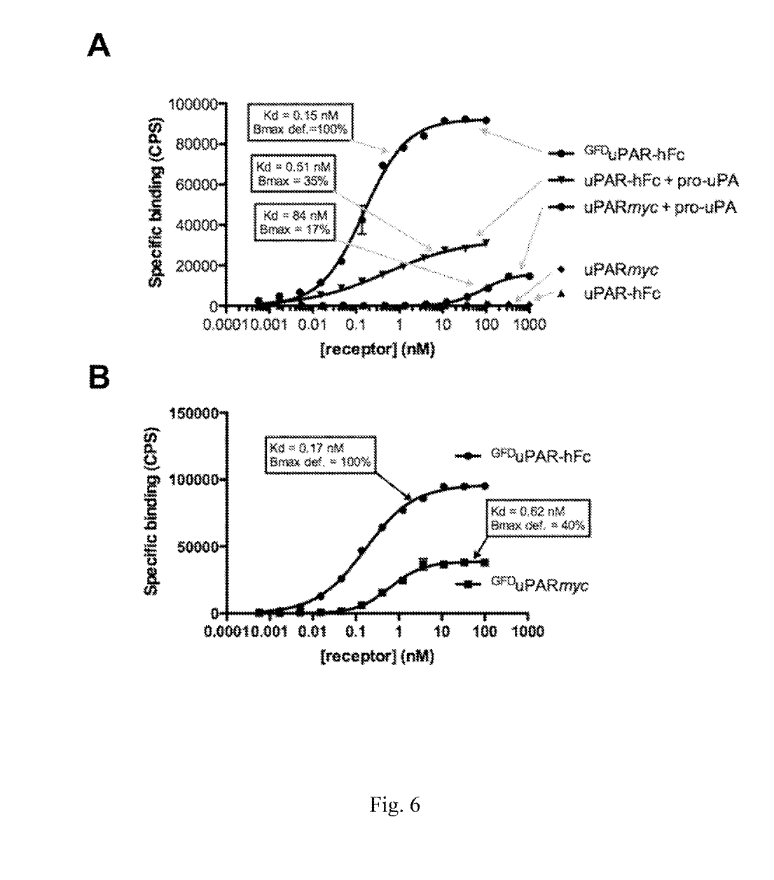

FIG. 6: Direct comparison of VN-binding activity of different forms of soluble uPAR (A) GFDuPAR-hFc binds with high affinity to immobilized VN. 96-well plates coated with VN were incubated with increasing concentrations of GFDuPAR-hFc in the absence of pro-uPA or uPAR-hFc and uPARmyc in the presence and absence of excess pro-uPA. After washing, the bound receptor was detected by sequential incubations with a monoclonal uPAR antibody (13F6) and a Eu3+-labeled goat-anti mouse antibody. The data are from a single experiment and presented as means±SD. The binding curve and Kd were calculated by non-linear regression. Note that GFDuPAR-hFc binds VN with higher affinity and capacity than any other form of uPAR tested. (B) Comparison of GFDuPAR-hFc and GFDuPARmyc binding to immobilized VN. 96-well plates coated with VN were incubated with increasing concentrations of GFDuPAR-hFc and GFDuPARmyc in the absence of pro-uPA for 2 hours at room temperature. After washing, the bound receptor was detected by sequential incubations with a monoclonal uPAR antibody (13F6) and a Eu3+-labeled goat-anti mouse antibody. The data are from a single experiment and are represented as means±SD. The binding curve and Kd were calculated by non-linear regression. Note that the dimeric GFDuPAR-hFc binds VN with higher affinity (˜4-fold) and capacity (˜2.5-fold) than the monomeric GFDuPARmyc.

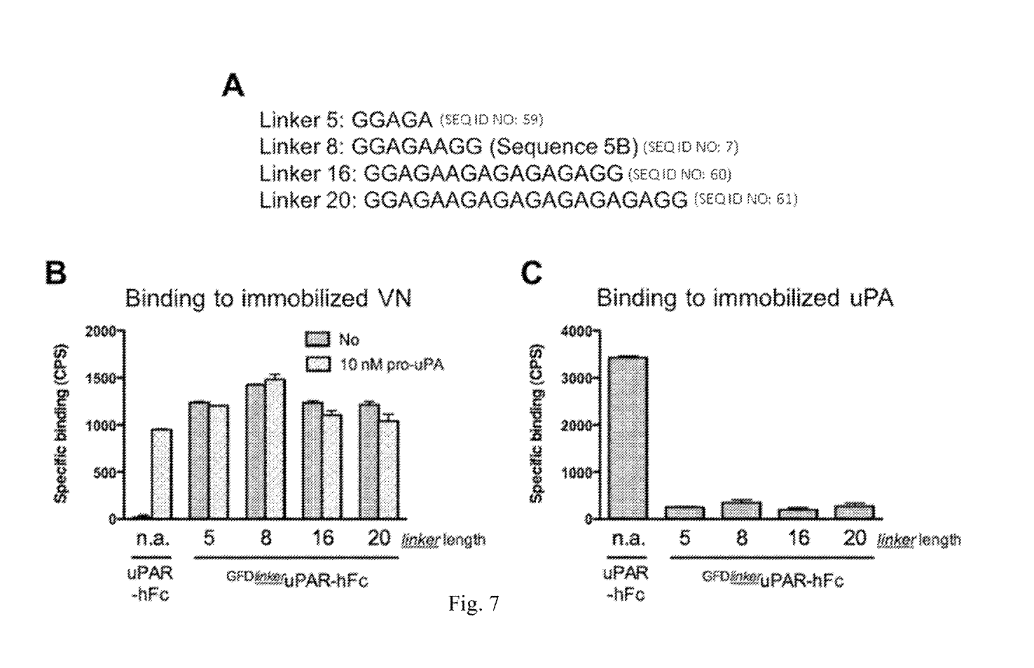

FIG. 7: Lack of specific requirements to the linker region connecting the GFD and uPAR domains in uPAR-hFc (A) Tested linker regions. To compare the possible effect of different linker length between the GFD and uPAR domains in GFDuPAR-hFc (see FIG. 5A), variants of GFDuPAR-hFc were made with the indicated linker sequences 5, 8, 16 or 20 residues long. The Linker 8 is identical to Sequence 5B (SEQ ID NO: 7) and to Sequence 9B (SEQ ID NO: 8) used in all the above experiments. (B) Binding to immobilized VN. Wells coated with VN were incubated with conditioned medium (diluted 10-fold) from 293 cell transiently transfected with the indicated uPAR variants in the presence or absence of 10 nM pro-uPA. After washing, the bound material was detected by incubation with a Eu3+-labeled anti-human Fc antibody and measurement of time-resolved fluorescence. Note that independently of the linker length, all the GFDuPAR-hFc variants bind to VN independently of pro-uPA. (C) Binding to immobilized uPA. Wells coated with pro-uPA were incubated with conditioned medium (dilute 10-fold) from 293 cell transiently transfected with the indicated uPAR variants. After washing, the bound material was detected as described in Panel B. Note that independently of the linker length all the GFDuPAR-hFc variants display reduced binding to uPA as compared to uPAR-hFc.

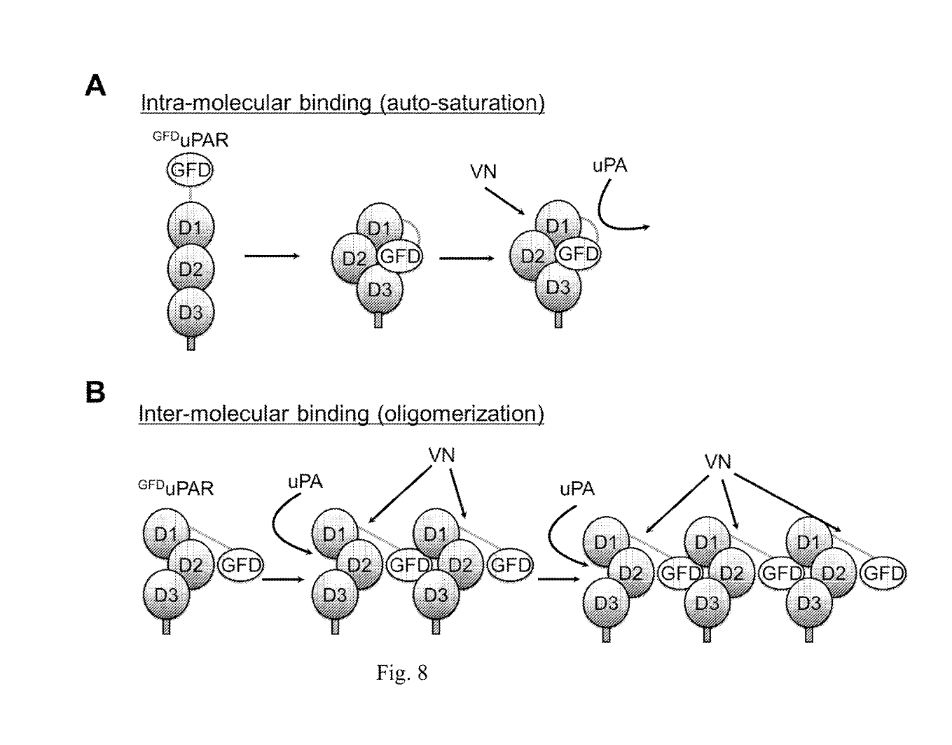

FIG. 8: Cartoon illustrating two possible mechanisms by which the appending of GFD on uPAR may increase VN-binding and reduce uPA-binding. (A) Intra-molecular binding. If sterically allowed, the GFD-domain in GFDuPAR may bind to the uPA-binding pocket in uPAR leading to auto-saturation of the chimeric receptor. Auto-saturation of uPAR may induce a conformational change in the receptor leading to the efficient exposure of the VN binding site and prevent binding of uPA. (B) Inter-molecular binding. If auto-saturation as shown in panel A is not possible for sterical reasons, GFDuPAR will be hetero-divalent and thus display both uPA and uPAR binding activity. In this case, GFDuPAR is likely to form oligomers displaying reduced uPA binding activity and increased VN binding activity.

FIG. 9: Amino acid sequence of 8 antibody variable regions. The amino acid sequence of the variable regions of the heavy and light chains were deduced from the cDNA sequence obtained by PCR amplification as described in the materials and methods. The amino acid sequences are numbered according to the Kabat system. The complementarity determining regions (CDR) 1, 2 and 3 (from left to right) are underlined. Gaps introduced in the sequences to maintain alignment are indicated by hyphens. Punctuation, which corresponds to an Xaa in the sequence listing, indicates that the sequence is either unknown or uncertain. Thus, Xaa can be any naturally occurring amino acid.

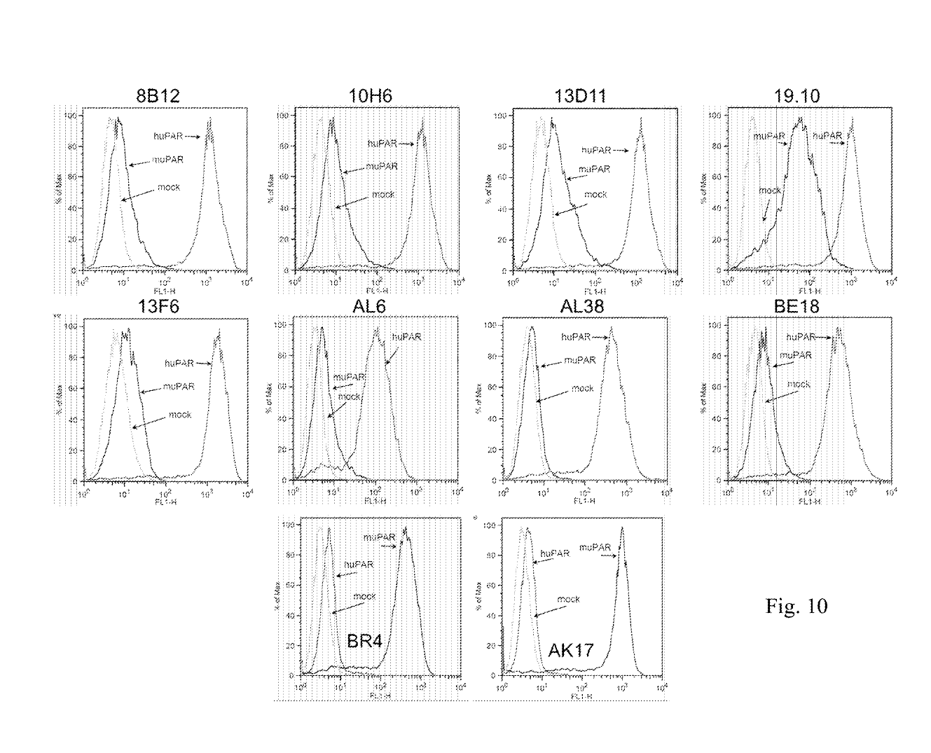

FIG. 10: Monoclonal antibodies raised against GFDuPAR-hFc recognize cell surface uPAR. 293 cells expressing human uPAR (huPAR), mouse uPAR (muPAR) or no uPAR (mock) were stained with the monoclonal antibodies 8B12, 10H6, 13D11, 19.10, 13F6, AL6, AL38 and BE18 raised against GFDuPAR-hFc. Bound antibody was detected using a fluorescein labeled goat anti mouse antibody and the staining was analyzed by flow cytometry. The histograms show the staining intensity (X-axis, FL1-H) and frequency (Y-axis, in % of the most frequent intensity). Note that all eight antibodies stain cells expressing human uPAR specifically. The antibodies BR4 and AK17 have been described previously and react specifically with mouse uPAR (Tjwa et al., 2009).

FIG. 11: Functional inhibitory activity of mAb 8B12. 293 cells expressing human uPAR were seeded in 96-well E-plates coated with Vitronectin (A and B) or Fibronectin (C) and transferred to a real time cell analyzer instrument (RTCA, xCELLigence, SP Roche Corp.). The electric impedance (termed cell index, CI) was recorded at regular intervals. After approximately 2 hours, cells were added pro-uPA (B and C) or vehicle (A) and the cell index measurements continued. About one additional hour later, wells were added a dilution curve of 8B12 antibody at the final concentrations indicated in the graphs and the cell index measurements continued. The times at which pro-uPA and 8B12 were added are indicated in the graphs by stippled vertical lines. The curves show the normalized cell index (NCI, Y-axis) as a function of time (X-axis). All cell indexes were normalized to the cell index measured immediately prior to antibody addition. To determine IC50 values (panel D), the NCI measured one hour after antibody addition were calculated in % of the NCI for untreated cells at the same time point (ΔNCI, Y-axis) and graphed in function of antibody concentration (X-axis).

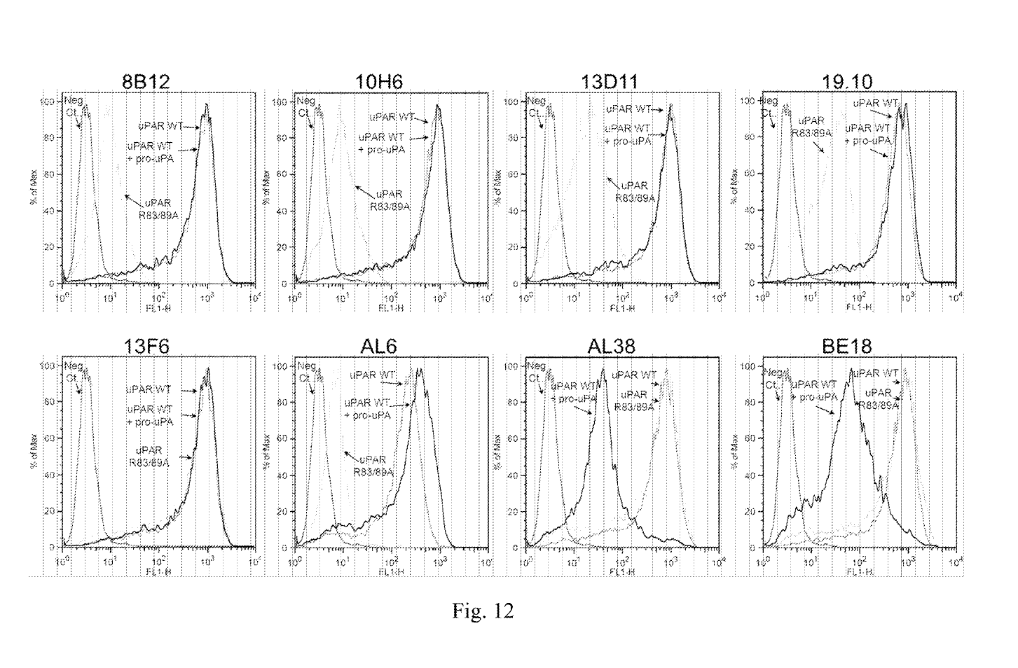

FIG. 12: Epitope mapping by flow cytometry (I). 293 cells expressing human uPAR (uPAR WT), uPAR R83/89A were stained with the different antibodies as indicated and the binding analyzed by flow-cytometry. The staining of uPAR WT cells was conducted both in presence and absence of pro-uPA to detect possible effects of ligand occupancy on antibody binding. As negative control (Neg. Ct.), the staining profile of uPAR WT cells receiving no primary antibody is shown in all panels.

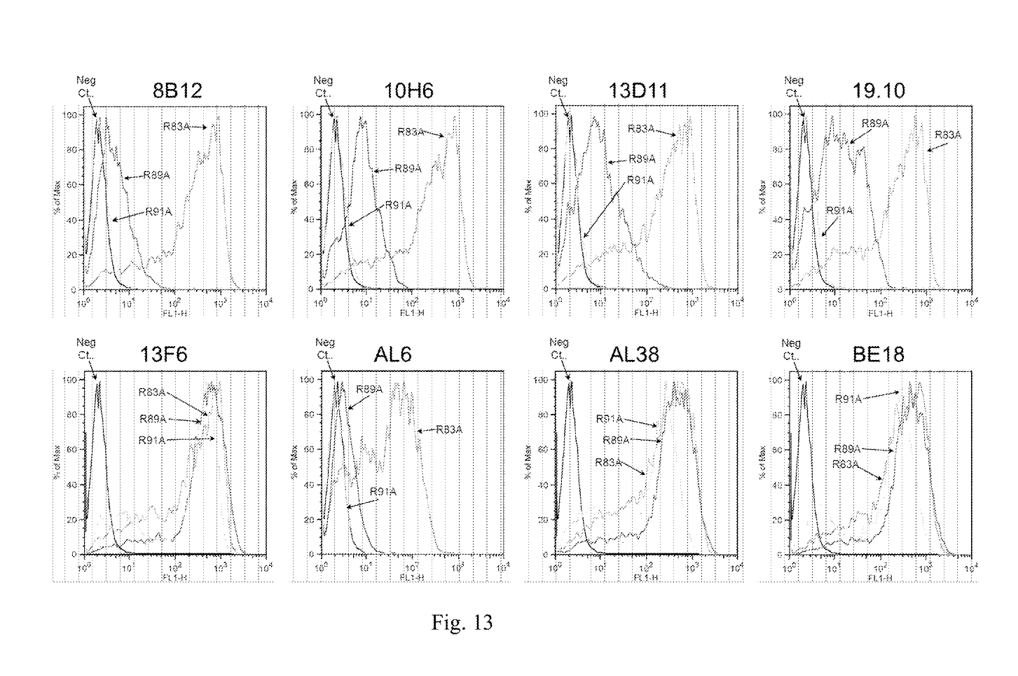

FIG. 13: Epitope mapping by flow cytometry (II). As FIG. 12 but different uPAR variants analyzed.

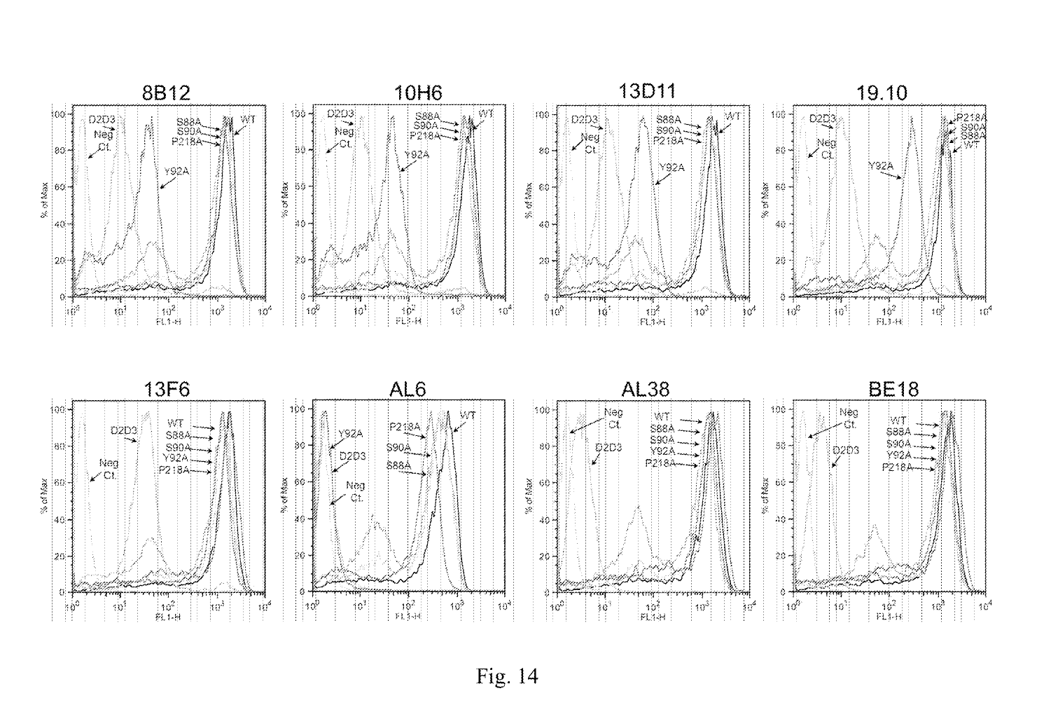

FIG. 14: Epitope mapping by flow cytometry (III). As FIGS. 12 and 13 but with different uPAR variants analyzed.

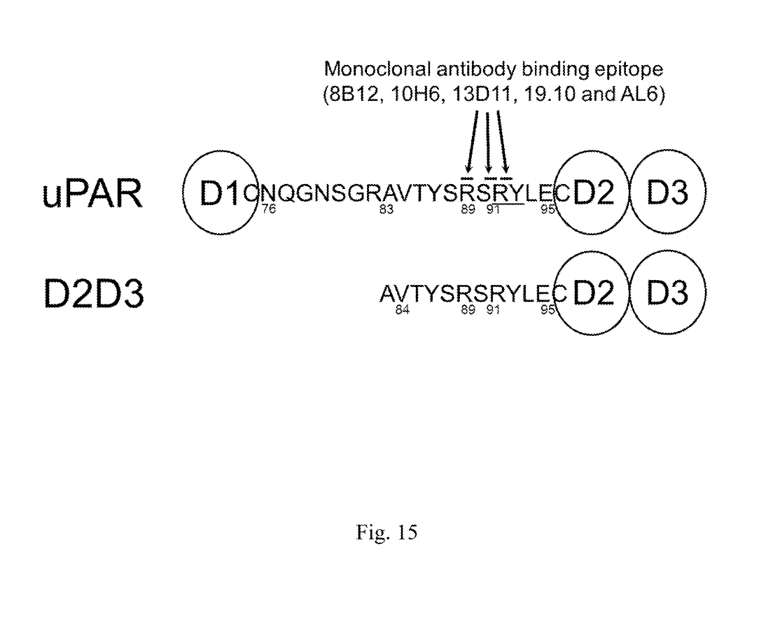

FIG. 15: Location of the binding epitope for the inhibitory antibodies in uPAR. uPAR is composed of three domains (D1, D2 and D3) where D1 is linked to D2 by a short linker region. This linker region contains residues that are critical for receptors interaction with VN (R91 and Y92, underlined) (Madsen et al., 2007) (Gardsvoll and Ploug, 2007). The binding site for the inhibitory antibodies generated in this example has overlapping epitope(s) with R89, R91 and Y92 being important hot-spots for binding. The structure of the D2D3 truncation version of uPAR is shown below. This variant lacks residues 1-82 of uPAR of SEQ ID NO: 1.

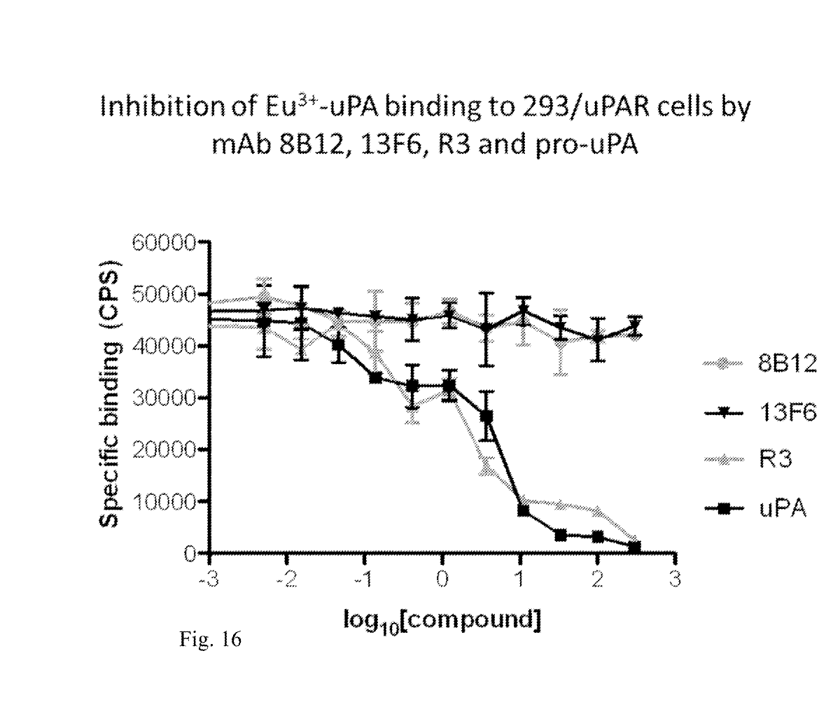

FIG. 16: Inhibition of Eu³⁺-uPA binding to 293/uPAR cells by mAb 8B12, 13F6, R3 and pro-uPA. mAb 8B12 does not interfere with the proteolytic functions of uPAR. To investigate if the inhibitory antibody 8B12 is a specific inhibitor of the uPAR/VN-interaction, or if it also interferes with uPA binding to the receptor, we conducted in vitro binding assays. Note that mAb 8B12 displays no or little inhibitory activity documenting that this antibody does not interfere with the proteolytic functions of the receptor. The validity of the assay is documented by the fact that the R3 antibody, and un-labeled pro-uPA, displayed efficient competitive activity. (CPS—Counts per second).

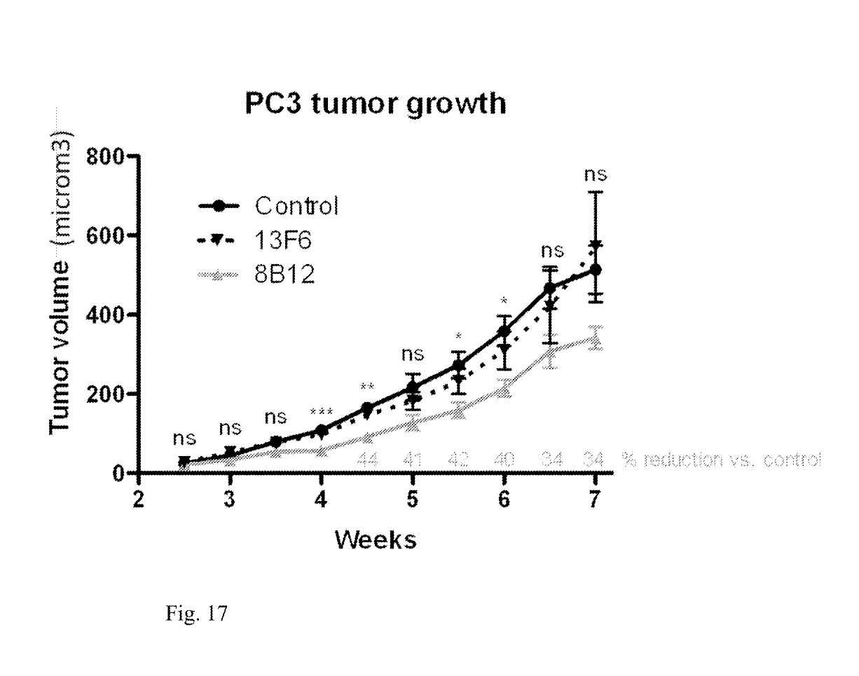

FIG. 17: mAb 8B12 inhibits PC3 tumor growth in vivo. Male Balb C nu/nu mice were inoculated with (1×10⁶) PC-3 cells through the subcutaneous (s.c.) route. Animals were treated by bi-weekly injections with 10.0 mg/kg of mAb 8B12, mAb 13F6, a non-immune control mouse IgG (mIgG) or PBS via intraperitoneal route. Tumors were measured twice weekly, and tumor volume was determined as described in Materials and Methods. No differences were observed in the tumor growth between PBS and mIgG treated animals and data from these were pooled prior to statistical analysis. Significant differences between control animals and 8B12 treated animals are represented by asterisks (NS, Non-Significant, P>0.05, *P<0.05, **P<0.01 and ***P<0.001). The difference in tumor volume between control and 8B12 treated animals (in %) is indicated.

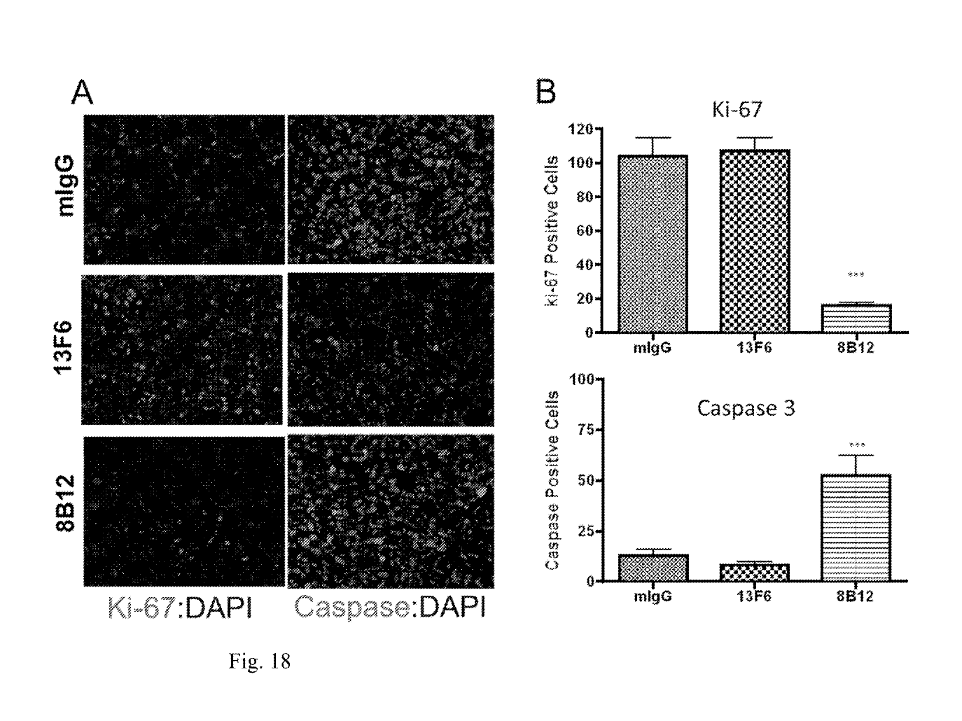

FIG. 18: mAb 8B12 reduces PC-3 tumor cell proliferation and promotes apoptosis in vivo. Male Balb C nu/nu mice were inoculated subcutaneously with PC-3 cells and treated by bi-weekly injections with 10.0 mg/kg of mAb 8B12, mAb 13F6 or a non-immune control mouse IgG via intraperitoneal route. Eight weeks after xenografting, the tumors were harvested and analyzed by immunohistochemistry (Panel A) as described in the Materials and Methods section. Ki-67 and activated Caspase-3 stainings are shown and nuclei are counterstained with DAPI. The quantification of the data is shown in Panel B. Note that the treatment with the inhibitory mAb 8B12 significantly reduces tumor cell proliferation and increases apoptosis when compared to treatment with control IgG. The non-inhibitory mAb 13F6, of the same isotype, does not display this activity documenting that it is the inhibitory activity of the mAb 8B12 that is responsible for the anti-proliferative and pro-apoptotic effect. The unit of the Y-axis is number of positive cells per field.

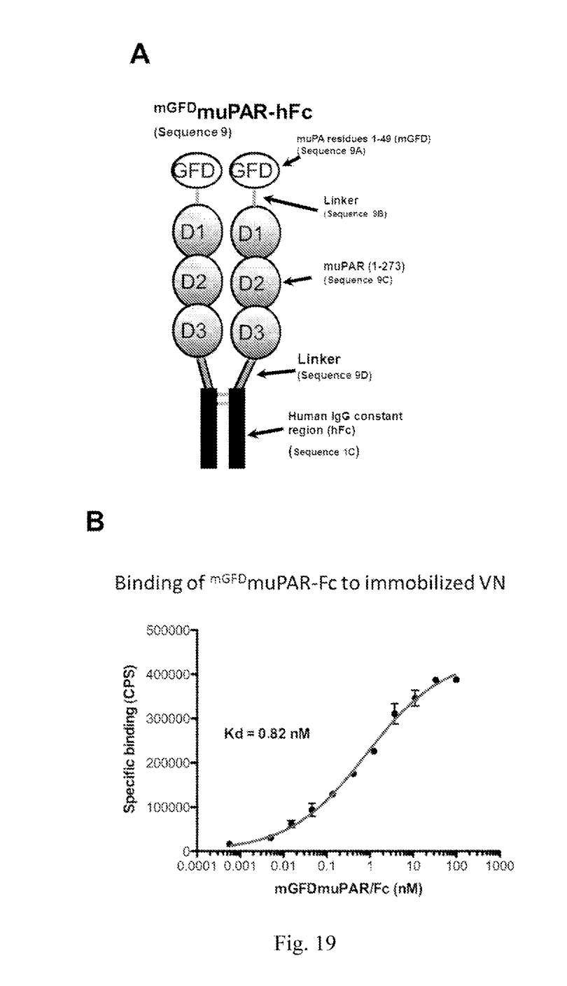

FIG. 19. Domain composition and VN-binding characteristics of ^{mGFD}muPAR-Fc (A) Cartoon illustrating the domain organization of ^{mGFD}muPAR-hFc. ^{mGFD}muPAR-hFc (Sequence 9 (SEQ ID NO: 17)) is composed of the receptor-binding growth factor-like domain of murine uPA, mGFD (Sequence 9A (SEQ ID NO: 4)), a short linker (Sequence 9B (SEQ ID NO: 8)), mouse uPAR residues 1-273 (Sequence 9C corresponding to aa. 1-273 of SEQ ID NO: 2), another short linker (Sequence 9D (SEQ ID NO: 11)) and a C-terminal human Fc-tag (hFc, Sequence 1C (SEQ ID NO: 5)). A C-terminal of mouse Fc-tag can be equally used.

(B) Binding of ^{mGFD}muPAR-hFc to immobilized VN. 96-well plates coated with VN were incubated with increasing concentrations of ^{mGFD}muPAR-hFc for 2 hours at room temperature. After washing, the bound receptor was detected by incubation with a ^{Eu3+}-labeled goat anti-human Fc antibody. The bound material was detected by measuring time-resolved fluorescence intensity. Specific binding was calculated by subtracting the non-specific binding measured in uncoated wells incubated with identical samples. The shown data are means±SD from a representative experiment. The dissociation constant (Kd) was calculated by non-linear regression (four-parameter fit) using the Prism 5.0 software suite.

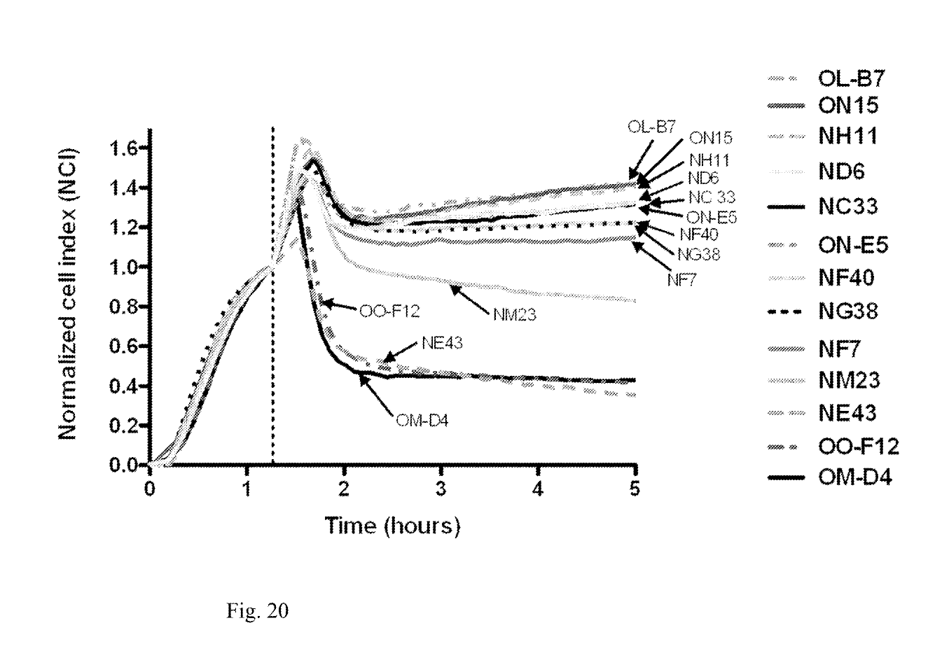

FIG. 20. Antibodies raised against ^{mGFD}muPAR-hFc inhibit cell adhesion to VN mediated by mouse uPAR. Inhibition of 293/muPAR cell adhesion to VN by cell culture supernatants from myeloma hybrids producing antibodies recognizing ^{mGFD}muPAR-hFc. 293 cells expressing murine uPAR were seeded in VN-coated E-plates and cell adhesion followed by impedance measurements using an xCELLigence plate reader (Roche). When adhesion arrived at plateau (indicate by stippled vertical line), the wells were added conditioned medium (final concentration 30% v/v) from the 13 different myeloma hybrids derived from splenocytes from mice immunized with ^{mGFD}muPAR-hFc. Note that the conditioned medium from 4 hybrids results in a strong (OMD4, NE43 and OOF12) or intermediate reduction (NM23) in cell adhesion (measured as the normalized cell index) while conditioned medium from the remaining 9 hybrids displays little or no inhibitory activity.

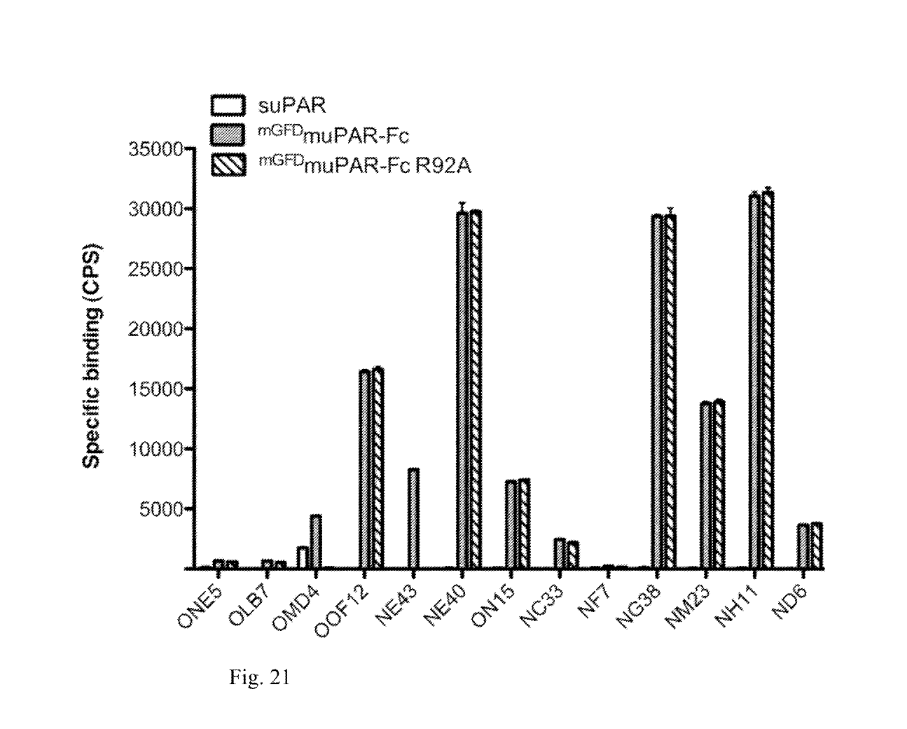

FIG. 21. The inhibitory antibodies OMD4 and NE43 bind to the VN binding site in mouse uPAR. To determine if the binding epitope of the generated antibodies falls in the VN-binding site of mouse uPAR (muPAR), in vitro binding assays were conducted on the antigen used for immunization (^{mGFD}muPAR-hFc) and a variant of this chimera containing a single amino acid substitution in the VN binding site of muPAR (^{mGFD}muPAR-hFc R92A) as well as a human soluble receptor (suPAR) to determine if the antibodies also recognize human uPAR.

96-well elisa plates were coated with ^{mGFD}muPAR-hFc, ^{mGFD}muPAR-hFc R92A or human soluble uPAR (suPAR), blocked and incubated with hybridoma supernatants diluted 1:100 in dilution buffer. After washing, bound antibody was probed by incubation with a Eu3+-labeled goat anti-mouse antibody and quantified by enhanced timeresolved fluorescence intensity measurements (Delfia). Specific binding was calculated by subtracting the binding observed to uncoated wells. Note that OMD4 and NE43 do not recognize the ^{mGFD}muPAR-hFc R92A variant suggesting that these antibodies bind to the VN binding site in muPAR. One of these antibodies (OMD4) also recognizes the human receptor.

FIG. 22. Amino acid sequence of mAb OMD4 raised against ^{mGFD}muPAR-Fc heavy chain variable region. The amino acid sequences of the variable region of the OMD4 heavy chain was deduced from the cDNA sequence obtained by PCR amplification as described in the Materials and Methods, Example 2. The amino acid sequence is numbered according to the Kabat system. The complementarity determining regions (CDR) 1, 2 and 3 (from left to right) are underlined.

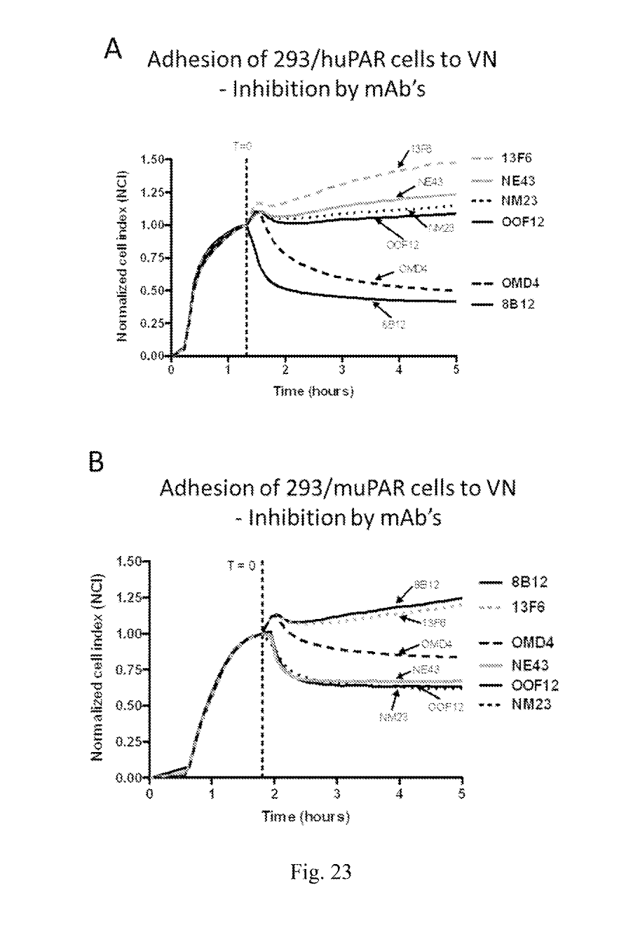

FIG. 23. Species specificity of the inhibitory activity of mAb OMD4, NE43, OOF12, NM23, 8B12. 293 cells expressing human uPAR (Panel A) and mouse uPAR (Panel B) were seeded on VN-coated E-plates and cell adhesion monitored by impedance measurement. Once a plateau of cell adhesion was reached (vertical stippled line), wells were added purified antibody to a final concentration of 100 nM* and the resulting changes in cell adhesion recorded. Note that the adhesion of cells expressing human uPAR is inhibited by mAb 8B12 and partially by mAb OMD4, while the remaining antibodies are without notable effect. In contrast, the adhesion of cells expressing murine uPAR is inhibited by mAb NE43, OOF12, NM23, partially by OMD4, but not at all by 8B12. 13F6 was used as a non-inhibitory negative control antibody binding human uPAR. *The OMD4 antibody is IgA isotype and was used in the form of cell culture supernatant diluted 1:5. The concentration of this antibody in the supernatant is unknown and may be low. The partial effect observed with this antibody may therefore be attributed to this.

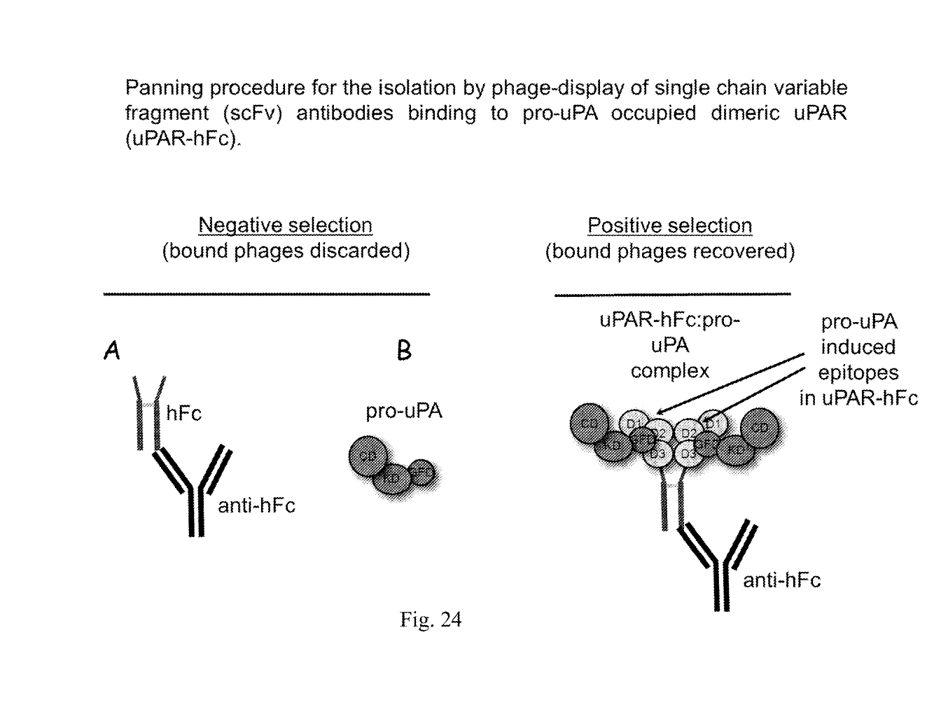

FIG. 24: Panning strategy for the isolation of scFv's recognizing ligand occupied dimeric uPAR

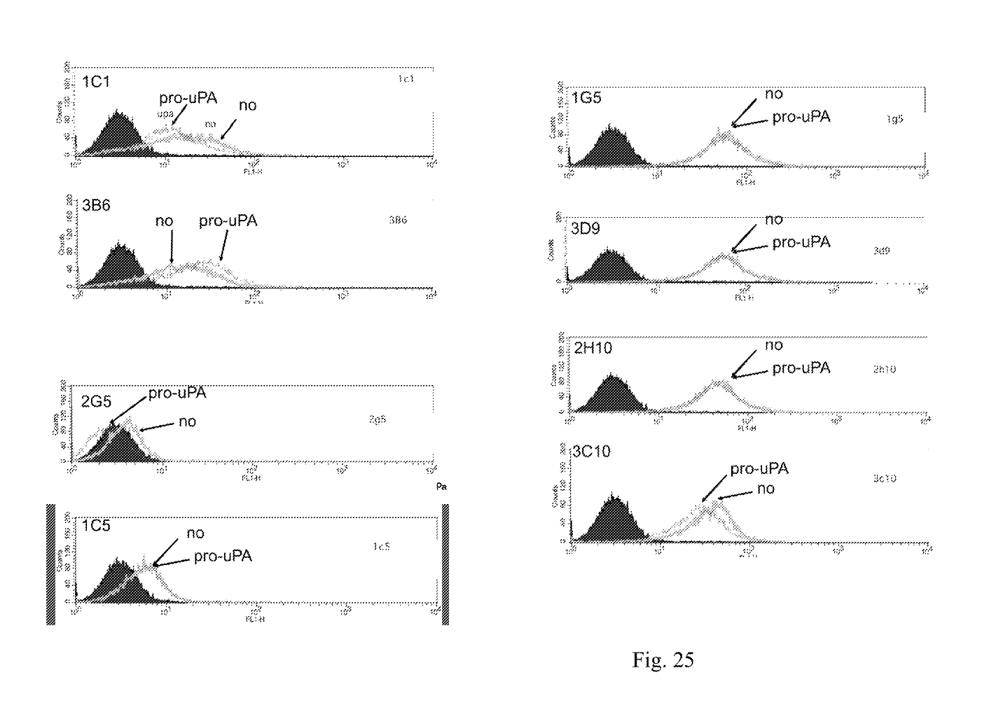

FIG. 25: Reactivity of isolated scFv with cell surface uPAR. 293 cells expressing human uPAR were stained with the indicated scFv (200 nM). Bound antibody was detected using a fluorescein labeled goat anti-human F(ab)2 antibody and the staining was analyzed by flow cytometry. The histograms show the staining intensity (X-axis, FL1-H) and frequency (Y-axis, in counts).

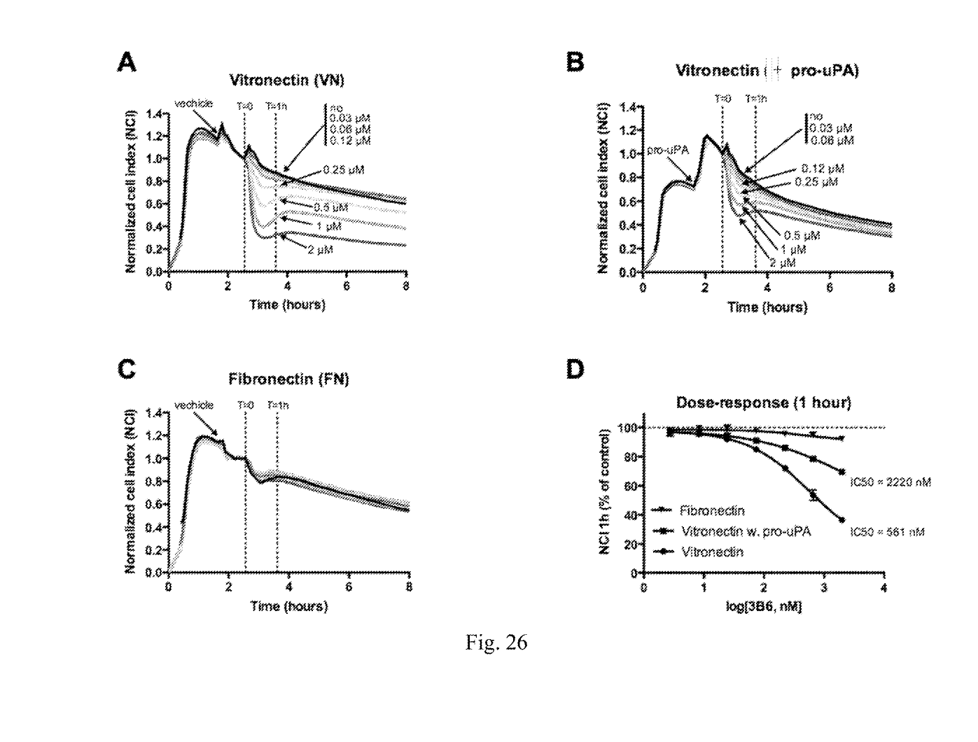

FIG. 26: Inhibitory activity of scFv 3B6. The inhibitory activity of scFv 3B6 was assayed as described for mAb 8B12 in FIG. 11. The curves show the normalized cell index (NCI, Y-axis) as a function of time (X-axis). All cell indexes were normalized to the cell index measured immediately prior to antibody addition. To determine IC50 values (panel D), the NCI measured one hour after antibody addition were calculated in % of the NCI for untreated cells at the same time point (ΔNCI, Y-axis) and graphed in function of antibody concentration (X-axis).

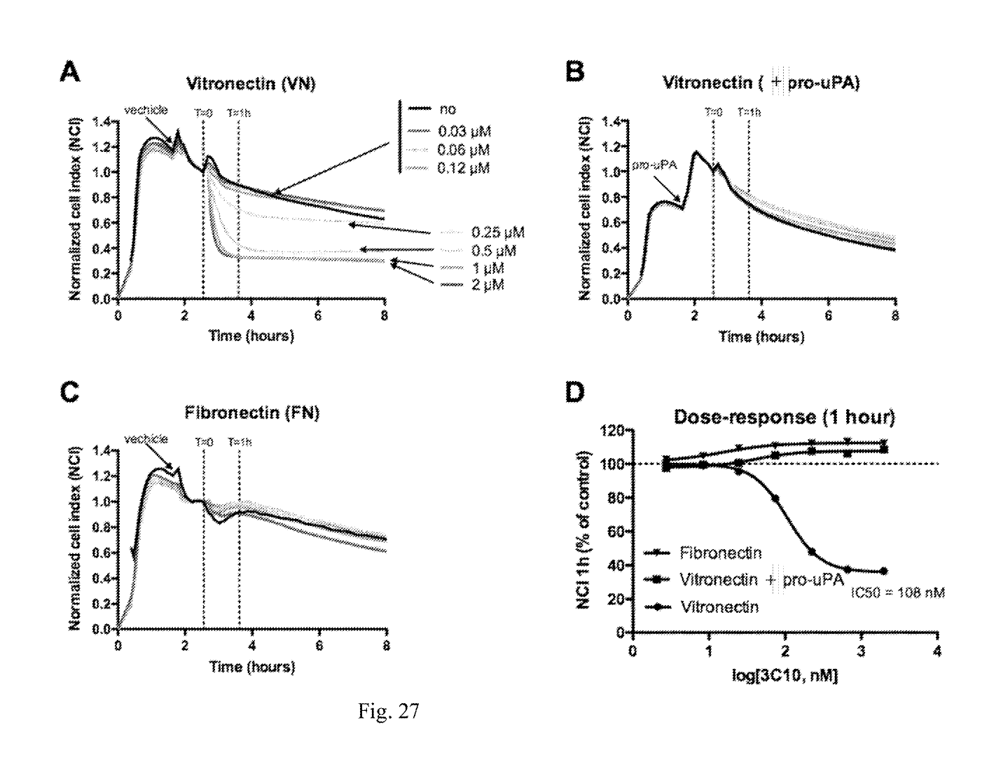

FIG. 27: Inhibitory activity of scFv 3C10. The inhibitory activity of 3C10 was assayed exactly as described for scFv 3B6 in FIG. 26.

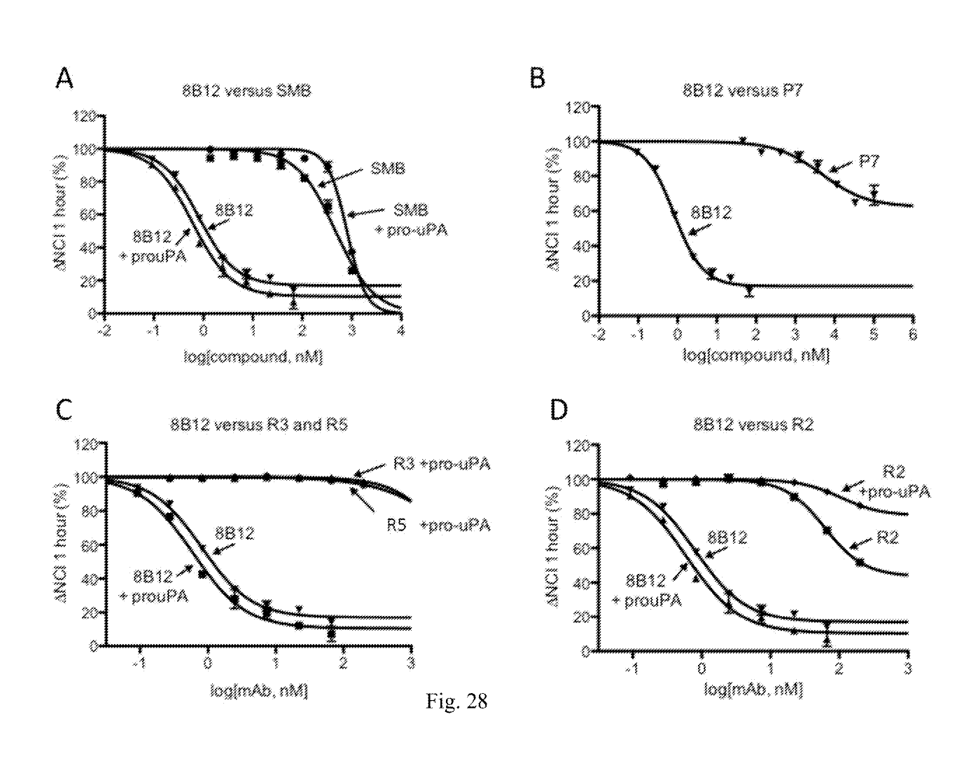

FIG. 28: Comparison of the inhibitory activity of 8B12 with that of other compounds known to inhibit the uPAR/VN-interaction or uPAR function. The inhibitory activity of the SMB domain (Panel A), the peptide P7 (Panel B), antibodies R3 and R5 (Panel C) as well as the R2 antibody (Panel D) were measured as described for the 8B 12 antibody in FIG. 11. To determine the IC50 values, the NCI measured one hour after compound addition were calculated in % of the NCI for untreated cells at the same time point (ΔNCI, Y-axis) and graphed in function of compound concentration (X-axis). The inhibition curves for 8B12 from FIG. 11 have been included in all four panels for comparison. The calculated IC50 and max inhibition constants for each of the tested compounds can be found in Table 2.

### EXAMPLE 1

### Materials and Methods

### Construction of Expression Vectors

The expression vectors for recombinant proteins tagged with a human IgG constant region (hFc) are based on the pFRT/TO-Fc plasmid (Madsen et al., 2007), however a number of modifications were introduced to facilitate the shuffling of different coding regions as well as to improve protein yields. Firstly, an XhoI restriction site located in the vector sequence downstream of the hFc coding region was destroyed by site-directed mutagenesis using oligos dXu/dXd. Secondly, a linker encoding a cleavage sequence for the PreScission protease, made by annealing oligos PreF/PreR, was inserted in the XhoI site located at the signal peptide/Fc junction. To remove the introns present in the Fc region of the construct, which was found to increase the yield of recombinant protein (our unpublished observations), the vector was transfected into CHO cells, RNA extracted, reverse transcribed, and the cDNA amplified with oligos hVNukpn/FcNr and cloned KpnI/NotI into pcDNA5/FRT-TO (Invitrogen corp.) and pEGFP-N1 (Clontech corp.) to generate pFRT/TO-hFc and pN1-hFc, respectively. Expression vectors for recombinant proteins tagged with a mouse IgG constant region (mFc) was generated by PCR amplification of a mouse IgG1 cDNA (clone IRAVp968B035D, obtained from imaGenes GmbH) with oligos mFcU/mFcD and cloned XhoI/NotI in pFRT/TO-hFc and pN1-hFc to generate pFRT/TO-mFc and pN1-mFc, respectively. Constructs encoding soluble uPAR tagged with a human Fc (uPAR-hFc, Sequence 1 (SEQ ID NO: 14)) and mouse Fc (uPAR-mFc, Sequence 2 (SEQ ID NO: 15)) were made by amplification of a full-length uPAR cDNA (Madsen et al., 2007) with oligos URskF/UpreR2D and cloned KpnI/XhoI into pFRT/TO-hFc and pFRT/TO-mFc to generate pFRT/TO-uPAR-hFc and pFRT/TO-uPAR-mFc, respectively. The construct encoding soluble myc-tagged uPAR (uPARmyc, Sequence 3 (SEQ ID NO: 26)) was generated by amplification of the uPAR cDNA with oligos URskF/URMYCR and cloned KpnI/NotI into pcDNA5/FRT-TO to generate pFRT/TO-uPARmyc. The expression vector encoding a chimera between the growth factor domain of uPA (GFD, Sequence 5A (SEQ ID NO: 3)) and full-length uPAR (Sequence 4 (SEQ ID NO: 1)). ^{GFD}uPAR (Sequence 5 (SEQ ID NO: 16)) was generated in a two-step PCR overlap amplification procedure. Firstly, an uPA cDNA was amplified with oligos ATFkpnF/GFD1r and an uPAR cDNA with oligos UL8f/FO12394. Secondly, the two PCR products were mixed, co-amplified using oligos ATFkpnF/FO12394 and cloned KpnI/NotI in pcDNA5/FRT-TO to generate pFRT/TO-^{GFD}uPAR. The expression vector encoding soluble ^{GFD}uPAR with a C-terminal myc-tag (^{GFD}uPARmyc, Sequence 6 (SEQ ID NO: 28)) was generated by amplifying pFRT/TO-^{GFD}uPAR with oligos ATFkpnF/URMYCR and cloning the product KpnI/NotI in pcDNA5/FRT-TO to generate pFRT/TO-^{GDF}uPARmyc. The expression vectors encoding soluble dimeric ^{GFD}uPAR-variants with a C-terminal human Fc-tag (^{GFD}uPAR-hFc, Sequence 7 (SEQ ID NO: 12)) and mouse Fc-tag mFc (^{GFD}uPAR-mFc, Sequence 8 (SEQ ID NO: 13)) tags were generated by amplifying pFRT/TO-^{GFD}uPAR with oligos ATFkpnF/UpreR2D and cloning the product KpnI/XhoI in pFRT/TO-hFc and pFRT/TO-mFc to generate pFRT/TO-^{GDF}uPAR-hFc and pFRT/TO-^{GDF}uPAR-mFc, respectively. Expression vectors encoding chimeras with different lengths of linker region between the GFD and uPAR domains in the chimera were made as described above replacing oligo uL8f with uL5f, uL12f, uL16f or uL20f. The region encoding ^{GFD} uPAR-hFc, and its variants with different linker length, were transferred KpnI/NotI to the pEGFP-N1 expression vector (Clontech Corp.) generating pN1-^{GFD}uPAR-hFc used for transient expression experiments.

### Expression and Purification of Recombinant Proteins

The pFRT/TO-uPAR-hFc, pFRT/TO-^{GFD}uPAR-hFc, pFRT/TO-uPAR-mFc, pFRT/TO-^{GFD}uPAR-mFc, pFRT/TO-uPARmyc pFRT/T 0-^{GFD}uPARmyc, expression vectors were transfected into CHO Flp-In cells (Invitrogen Corp.) and the recombinant proteins expressed under serum-free conditions as previously described (Madsen et al., 2007). Recombinant tagged with human or mouse Fc tags were purified from the conditioned media by standard Protein A affinity chromatography and dialyzed extensively against PBS. The conditioned medium of pFRT/TO-uPARmyc and ^{GFD}uPARmyc transfected cells was concentrated ˜20-fold and utilized for binding assays without further purification. Standard ELISA assays were employed to determine the concentrations of uPARmyc in the concentrated conditioned media. The ^{GFD}uPAR-hFc variants with different lengths of linker between the GFD and uPAR moiety were expressed by transient transfection of Phoenix cells cultured in OptiMEM serum-free media (Invitrogen Corp.) with the pN1-^{GFD}uPAR-hFc vector variants and the conditioned medium recovered after 6-8 days of culture.

### Binding Assays

Black 96-well immunoplates were coated with pro-uPA or VN (10 nM) diluted in coating buffer (50 mM sodium carbonate, pH 9.6) at 4° C. ON. Plates were washed with wash buffer (phosphate buffered saline containing 0.1% Tween-20 (PBS-T) and non-specific binding sites saturated with blocking buffer (PBS containing 2% bovine serum albumin (BSA)) for >2 hours at RT. After washing with PBS-T, wells were incubated with the indicated concentrations of uPAR-hFc, uPAR-mFc and uPARmyc diluted in dilution buffer (PBS containing 1% BSA) in the presence or absence of pro-uPA as indicated. The binding was allowed to occur for 2 hours at RT after which unbound reagents were removed by rinsing with wash buffer. Bound uPAR-hFc and uPARmyc were detected by sequential incubations with an anti-uPAR monoclonal antibody (13F6, 1 μg/ml) and a Eu³⁺-labeled goat-anti mouse antibody (1:5.000, Perkin Elmer Corp.). The Eu³⁺-label was detected by measuring time-resolved fluorescence intensity using an Envision Xcite plate reader (Perkin Elmer Corp.) employing the DELFIA label protocol. To calculate the specific binding, non-specific binding measured in uncoated wells, incubated with identical samples, was subtracted from the total binding measured in coated wells.

### Cell Lines and Cell Adhesion Assays

HEK293 Flp-In T-REx cells (293) expressing ^{GFD}uPAR were generated by transfection with the pFRT/TO-^{GFD}uPAR vector according to published procedures (Madsen et al., 2007). 293 cells transfected with empty vector and cells expressing uPAR^{W32A} and uPAR^{R91A} have been described previously (Madsen et al., 2007).

### Oligonucleotide Sequences

dXu:
(SEQ ID NO: 29)
5′-gtaaatgagcggccgcgtcgagtctagaggg-3′
dXd:
(SEQ ID NO: 30)
5′-ccctctagactcgacgcggccgctcattta-3′
PreF:
(SEQ ID NO: 31)
5′-tcgagctggaagttctgttccaggggccca-3′
PreR:
(SEQ ID NO: 32)
5′-agctacccggggaccttgtcttgaaggtcg-3′
hVNukpn:
(SEQ ID NO: 33)
5′-cggggtaccatggcacccctgaga-3′
FcNr:
(SEQ ID NO: 34)
5′-ttgcggccgctcatttacccggagacag-3′
mFcU:
(SEQ ID NO: 35)
5′-gcctcgaggcaggagcaggacccagggattgtggttgtaa-3′
mFcD:
(SEQ ID NO: 36)
5′-gcgcggccgctcatttaccaggagagtg-3′
URskF:
(SEQ ID NO: 37)
5′-gcgtcgacggtacccgccaccatgggtcacccgccgctgctg-3′
UpreR2D:
(SEQ ID NO: 38)
5′-gcctcgaggggcccctggaacagaacttccagatccaggtctgggt
ggttacagccact-3′
URMYCR:
(SEQ ID NO: 39)
5′-gcgcggccgctcacagatcctcttcagagatgagtttctgctctcc
tcctgggtggttacagccact-3′
ATFkpnF:
(SEQ ID NO: 40)
5′-gcggtacccgccaccatgagagccctgctggcgcgc-3′
GFD1r:
(SEQ ID NO: 41)
5′-tgtgaaatagataagtcaaaagggggggccggggcg-3′
uL8f:
(SEQ ID NO: 42)
5′-gggggggccggggcggctggaggactgcggtgcatgcagtgtaag-3′
FO12394:
(SEQ ID NO: 43)
5′-tagtttagcggccgcttaggtccagaggagagt-3′
UpreR2D:
(SEQ ID NO: 44)
5′-gcctcgaggggcccctggaacagaacttccagatccaggtctgggt
ggttacagccact-3′
URMYCR:
(SEQ ID NO: 45)
5′-gcgcggccgctcacagatcctcttcagagatgagtttctgctctcc
tcctgggtggttacagccact-3′
uL5f:
(SEQ ID NO: 46)
5′-gggggggccggggcgctgcggtgcatgcagtgtaag-3′
uL16f:
(SEQ ID NO: 47)
5′-gggggggccggggcggctggagcaggagcaggtgctggtgctggag
gactgcggtgcatgcagtgtaag-3′
uL20f:
(SEQ ID NO: 48)
5′-gggggggccggggcggctggagcaggagcaggtgctggtgctggag
caggtgctggtggtctgcggtgcatgcagtgtaag-3′

### Results

Forced Dimerization of uPAR Strongly Enhances Binding to Vitronectin

### Background and Rationale

Several lines of evidence suggest that receptor-dimerization plays an important role for the interaction between uPAR and VN (Caiolfa et al., 2007; Sidenius et al., 2002), however, the dissociation constants determined by surface plasmon resonance (SPR) for the interaction between immobilized uPAR and soluble VN (˜1 μM, (Gardsvoll and Ploug, 2007)) are clearly insufficient to explain the high-affinity interaction predicted from equilibrium binding experiments using immobilized VN and soluble uPAR (Gardsvoll and Ploug, 2007; Sidenius et al., 2002). To directly address the role of uPAR-dimerization on VN-binding, authors here describe the construction, expression and purification of soluble forms of recombinant dimeric uPAR and the comparison of the ligand-binding characteristics of these with those of “conventional” soluble monomeric uPAR.

Construction and Expression of Dimeric uPAR

To directly determine the importance of receptor dimerization for the interaction between soluble uPAR and immobilized VN, authors constructed a soluble human uPAR tagged on the C-terminal with the hinge and constant region of a human IgG1 (hFc). The resulting uPAR-hFc chimera is a covalent homo-dimer in which the two polypeptides are held together by disulphide bonds located in the hinge region of the Fc-tag (FIG. 1A). The uPAR-hFc chimera (Sequence 1 (SEQ ID NO: 14)) is composed of uPAR (residues 1 to 277, Sequence 1A, corresponding to aa. 1-277 of SEQ ID NO: 1), a linker region (LEVLFQGPLE, Sequence 1B (SEQ ID NO: 9)) and the human Fc-tag (241 residues, Sequence 1C (SEQ ID NO: 5)). A similar construct was also made using the Fc-region of a mouse immunoglobulin and as illustrated in FIG. 1B the sequence and predicted domain structure of this chimera, uPAR-mFc (Sequence 2 (SEQ ID NO: 15)), is identical to that of uPAR-hFc with the exception of a slightly different linker region (Sequence 2A (SEQ ID NO: 10)) and the mouse Fc-tag (Sequence 2B (SEQ ID NO: 6)). As a monomeric control receptor, authors constructed a soluble human uPAR with a C-terminal myc-tag (uPARmyc, Sequence 3 (SEQ ID NO: 26)) illustrated in FIG. 1C. This protein is composed of uPAR (residues 1 to 274, Sequence 3A corresponding to aa. 1-274 of SEQ ID NO:1) and a C-terminal myc-tag (GGEQKLISEEDL, Sequence 3B (SEQ ID NO: 27)). The recombinant proteins were expressed in Chinese hamster ovary (CHO) cells and purified from the conditioned media by standard Protein A affinity chromatography (uPAR-hFc and uPAR-mFc) or utilized without purification (uPARmyc) after quantification by ELISA.

Binding Characteristics of Dimeric uPAR

The VN-binding activity of uPAR-hFc (FIG. 2A) and uPARmyc (FIG. 2B) were measured by incubating immobilized VN with increasing concentration of the recombinant receptors in the presence or absence of an excess of pro-uPA (the catalytically inactive zymogen form of uPA). After washing, bound receptor was revealed by sequential incubations with a mouse monoclonal anti-uPAR antibody (13F6), an Eu³⁺-labeled goat anti-mouse antibody and quantified by time-resolved fluorescence measurements. In the absence of pro-uPA, both uPAR-hFc and uPARmyc display poor binding to VN and the affinities of the interactions cannot be reliably estimated. However, in the presence of excess pro-uPA, both uPAR-hFc and uPARmyc display specific and dose dependent binding to VN. By non-linear regression analysis of the binding curves, the apparent dissociation constants (Kd) of the interaction between uPAR-hFc, uPARmyc and immobilized VN were calculated to be ˜10 nM and ˜80 nM, respectively. In contrast, both the monomeric and dimeric soluble receptors bind immobilized uPA with comparable apparent affinities of ˜6 nM (uPAR-hFc) and ˜10 nM (uPARmyc).

These data document that forced dimerization of uPAR, using an immunoglobulin Fc-tag, results in a ˜10-100-fold increase in the receptors apparent affinity for VN as compared to the monomeric receptor. The binding of both monomeric (uPARmyc) and dimeric (uPAR-hFc and uPAR-mFc) soluble uPAR is dependent upon concomitant occupancy by uPA as no or little binding is observed in its absence. The fact that forced dimerization of uPAR fails to increase the binding of the receptor to immobilized VN in the absence of uPA, as well as to immobilized uPA, suggests that the increase in apparent affinity involves unique conformational changes and that it is not only a result of increased avidity.

Chimeras Between uPA and uPAR Display Strong VN-Binding and Reduced uPA-Binding

### Background and Rationale

As described in the literature, and as illustrated in FIG. 2, binding of soluble uPAR to VN is strongly dependent upon the concomitant occupancy of the receptor by uPA. A plausible explanation for this observation is that uPA-binding to uPAR induces a conformational change in the receptor leading to the exposure of the VN-binding epitope in the occupied receptor. With the aim of generating an uPAR-variant displaying constitutive, i.e. uPA-independent, VN-binding combined with deficient uPA-binding, authors conceived that this could be achieved through the construction of an appropriate uPA/uPAR-chimera in which the intra-molecular binding reaction predicted to occur in such a chimera would lead to the exposure of the VN-binding epitope as well as prevent the binding of uPA in trans.

Construction of the uPA/uPAR chimera ^{GFD}uPAR

To generate an uPAR-variant constitutively active in VN-binding and deficient in uPA-binding, authors engineered the growth factor-like domain of uPA (GFD) onto the N-terminal of human uPAR as illustrated in FIG. 3A. The resulting chimera (^{GFD}uPAR, Sequence 5 (SEQ ID NO: 16)) is composed of the growth factor-like domain from human uPA (Sequence 5A (SEQ ID NO: 3)) connected by a short linker (Sequence 5B (SEQ ID NO: 7)) to the N-terminal of intact mature human uPAR (Sequence 4 (SEQ ID NO: 1)).

Binding Characteristics of Cell-Surface ^{GFD} uPAR

To analyze the binding characteristics of ^{GFD}uPAR chimera, authors generated 293 cell lines expressing ^{GFD}uPAR on the cell surface and compared their adhesion characteristics with that of cells expressing wild-type uPAR and uPAR-variants with specific deficiency in VN-binding (FIG. 3). In these assays, authors found that ^{GFD}uPAR, like the wild-type receptor, promotes firm, integrin-independent, cell adhesion to VN (FIG. 3B) confirming that the chimera retains full VN-binding activity. In addition, expression of ^{GFD}uPAR failed to promote cell binding to immobilized uPA suggesting that also the predicted loss of uPA-binding activity was attained in this chimera (FIG. 3C).

Construction and Binding Characteristics of Soluble ^{GFD}uPAR

uPAR-mediated cell adhesion to VN does not require uPA-binding as long as on the cell-surface expression levels are sufficiently high (Madsen et al., 2007; Sidenius and Blasi, 2000) and authors therefore next generated a soluble variant of ^{GFD}uPAR for in vitro binding experiments. For this purpose, authors constructed a truncated version of ^{GFD}uPAR carrying a C-terminal myc-tag in place of the membrane anchoring sequence of wild-type uPAR. The constructed chimera illustrated in FIG. 4A (^{GFD}uPARmyc, Sequence 6 (SEQ ID NO: 28)) has the same N-terminal GFD-domain (Sequence 5A (SEQ ID NO: 3)) and linker-sequence (Sequence 5B (SEQ ID NO: 7)) as described above for ^{GFD}uPAR and the same uPAR sequence (Sequence 3A (corresponding to aa. 1-274 of SEQ ID NO: 1)) and C-terminal myc-tag (Sequence 3B (SEQ ID NO: 27)) as described for uPARmyc in FIG. 1C.

The ^{GFD}uPARmyc chimera was produced in CHO cells and its binding characteristics analyzed by in vitro binding assays to immobilized VN (FIG. 4B) or immobilized pro-uPA (FIG. 4C). As presented, the ^{GFD}uPARmyc chimera binds with high affinity (Kd˜1.3 nM) to immobilized VN in the absence of uPA. Assayed under identical conditions, the control receptor uPARmyc, lacking the N-terminal GFD-domain, fails to display any appreciable binding to immobilized VN even at concentrations up to 1 μM. Appending the GFD-domain of uPA on the N-terminal of uPAR thus increases the measured binding affinity of the receptor to VN by at least three orders of magnitude. When the same proteins (^{GFD}uPARmyc and uPARmyc) were tested for binding activity towards immobilized pro-uPA (FIG. 4C), authors found that the ^{GFD}uPARmyc chimera displayed a reduced binding affinity (˜30-fold) and capacity (˜5-fold) as compared to the control receptor (uPARmyc).

### Conclusions

These data demonstrate that appending the GFD-domain of uPA on the N-terminal of uPAR increases the affinity of the receptor for VN by more than three orders of magnitude.

Forced Dimerization and uPA:uPAR-Chimerism Synergize to Increase VN-Binding Activity

### Background and Rationale

As documented above, the apparent affinity of uPAR for VN can be strongly increased by forced dimerization or by appending the GFD-domain of uPA on the N-terminal of uPAR. Finally authors here document that the combination of forced dimerization with the appending of the GFD-domain synergize to increase the apparent affinity for VN.

Construction and Expression of Dimeric ^{GFD}uPAR

Expression vectors encoding dimeric uPAR containing a GFD domain on the N-terminal of uPAR were constructed by combining a C-terminal Fc tag as shown in FIG. 1A-B with the engineering of the GFD domain of uPAR onto N-terminal of uPAR as illustrated in FIG. 3A. The resulting chimeric proteins ^{GFD}uPAR-hFc (Sequence 7 (SEQ ID NO: 12)) and ^{GFD}uPAR-mFc (Sequence 8 (SEQ ID NO: 13)) are illustrated in FIGS. 5A and B respectively and are composed of the GFD domain (Sequence 5A (SEQ ID NO: 3)) of uPA, a linker region (Sequence 5B (SEQ ID NO: 7)), uPAR residues 1-277 (Sequence 1A corresponding to aa. 1-277 of SEQ ID NO: 1), another linker region (Sequence 1B (SEQ ID NO: 9) or Sequence 2A (SEQ ID NO: 10)) and a C-terminal Fc tag from a human (Sequence 1C (SEQ ID NO: 5)) or mouse (Sequence 2B (SEQ ID NO: 6)) IgG.

Binding Characteristics of Dimeric ^{GFD}uPAR-hFc and ^{GFD}uPAR-mFc

The ^{GFD}uPAR-hFc and ^{GFD}uPAR-mFc chimeras were expressed in CHO cells and purified from the conditioned medium by Protein A affinity chromatography. To determine the VN-binding properties of the recombinant receptors, authors first measured the binding of ^{GFD}uPAR-mFc to immobilized VN (FIG. 5C). As shown, ^{GFD}uPAR-mFc binds with very high affinity (˜20 pM) suggesting that forced dimerization (using an Fc-tag) and ligand auto-saturation (using the GFD domain) synergizes to increase the VN-binding activity of uPAR.

To more directly compare the VN-binding characteristics of the different forms of soluble uPAR, additional binding experiments were conducted (FIG. 6). When compared to uPAR-hFc in the presence of uPA, ^{GFD}uPAR-hFc has about 3-fold higher affinity and binding capacity. When compared to monomeric soluble uPAR (uPARmyc) in the presence of uPA, ^{GFD}uPAR-hFc displays about 600-fold higher affinity and 6-fold higher binding capacity. Both monomeric (uPARmyc) and dimeric (uPAR-hFc) forms of uPAR show no or little binding to VN in the absence of uPA and quantification of the differences in affinity is therefore difficult. However, when compared to the published values for the binding of VN to immobilized uPAR (1.3 μM, (Gardsvoll and Ploug, 2007)), ^{GFD}uPAR-hFc display about 10.000-fold higher affinity. When compared directly to ^{GFD}uPARmyc (FIG. 6B), ^{GFD}uPAR-hFc display 3-fold higher affinity and 2.5-fold higher binding capacity demonstrating that both dimerization and ligand auto-saturation contributes to the remarkable binding activity of ^{GFD}uPAR-hFc and presumably also ^{GFD}uPAR-mFc.

Requirements to the Linker Region Connecting the GFD to uPAR

To determine the importance of the length and sequence of the linker region connecting the GFD domain to the N-terminal of uPAR in the ^{GFD}uPAR-hFc chimera, authors generated variants of this with a shorter linker (5 residues) and longer linkers (16 and 20 residues) and compared the VN and uPA binding activities with that of the “standard” linker (8 residues) used elsewhere in this study (FIG. 7A). The variants were expressed by transient transfection in 293 cell line and the binding activity in the conditioned medium was measured (FIG. 7). As shown, the addition of the GFD domain on the N-terminus of uPAR enhances VN binding (FIG. 7B) and reduces uPA binding (FIG. 7C) independently on the linker length applied suggesting that this sequence is very flexible in terms of length.

Possible Mechanisms Explaining the High Affinity of ^{GFD}uPAR Chimeras for VN

The concept behind the construction of ^{GFD}uPAR was that the GFD domain engineered onto the N-terminus of uPAR would bind to the uPA binding cavity in uPAR in an intra-molecular fashion as illustrated in FIG. 8A. Nevertheless, it is possible that the intra-molecular binding is prohibited by sterical constrains. In this case a single molecule of ^{GFD}uPAR may effectively display both uPAR and uPA binding activity and is likely to self-associate and oligomerize as shown in FIG. 8B.

### EXAMPLE 2

### Materials and Methods

### Antigen Preparation

^{GFD}uPAR-hFc and ^{GFD}uPAR-mFc were expressed and purified as described in detail in

### EXAMPLE 1

### Immunization of Mice

Three 2-month-old male C57Bl/6 uPAR^{-/-} mice (Ms^{#}21574, Ms^{#}1416 and Ms^{#}1417) were immunized by intraperitoneal (i.p.) injection with 67 μg ^{GFD}uPAR-hFc in 200 μl of a 1:1 emulsion between 100 μl immunogen in PBS and 100 μl of Complete Freund's Adjuvant (CFA). The immunized animals were boosted 3 times, at 3-week intervals, by IP injection of 67 μg ^{GFD}uPAR-hFc in 200 μl of a 1:1 emulsion between 100 μl immunogen in PBS and 100 μl of Incomplete Freund's Adjuvant (IFA). After a 7-weeks rest period, and 4 days before the fusion, Ms^{#}21574 was subjected to a final pre-fusion boost using 200 μg ^{GFD}uPAR-hFc in 200 μl PBS i.p. At 3-week intervals, Ms^{#}1416 and Ms^{#}1417 received three additional IP boosts with 34 μg ^{GFD}uPAR-hFc in 200 μl of a 1:1 PBS/IFA emulsion. After a 7-week rest period, 3 and 4 days before the fusions, Ms^{#}1416 and Ms^{#}1417 received a final pre-fusion boost using 250 μg ^{GFD}uPAR-mFc in 200 μl PBS i.p. plus 250 μg ^{GFD}uPAR-mFc in 200 μl PBS subcutaneously.

### Fusion and Hybridoma Culture and Cloning

Spleens were removed and the splenocytes fused to the mouse SP2/0 myeloma cell line by the polyethylene glycol method using standard procedures (Galfre et al., 1977). After fusion, the cells were cultured for one day in non-selective medium (Iscove, 10% FBS, 1×HFCS) and then plated in 96-well plates (35000 splenocytes/well) in selective HAT medium (Iscove, 1×HAT) supplemented with 1×HFCS (Hybridoma Fusion and Cloning Supplement, Roche Corp.). Hybridomas positive for the production of immunoglobulin specific for the antigen were identified by ELISA (see below), expanded in 12-well plates and frozen. Selected hybridomas were sub-cloned by limiting dilution in 96-well plates. Cells were plated in HT medium (Iscove, 1×HT) supplemented with 1×HFCS at a density ranging from 0.4 to 0.1 cells/well. When necessary, the sub-cloning procedure was repeated until all sub-clones scored positive by ELISA. The isotypes of the immunoglobulin produced by the different hybridomas were determined using a commercially available ELISA kit (Mouse Immunoglobulin Isotyping ELISA Kit, BD Pharmingen Corp.).

### Screening of Supernatants

Transparent 96-well plates (MAXI-SORP, NUNC Corp.) were coated with ^{GFD}uPAR-hFc (1 μg/ml in 0.1 M sodium carbonate buffer, pH 9.5). After washing with PBST (PBS containing 0.1% Tween-20), the wells were blocked with 3% BSA in PBST, washed and incubated with cell culture supernatants diluted 1:2 in PBST. Bound mouse immunoglobulin was detected using a peroxidase-conjugated goat-anti-mouse antibody followed by washing and colorimetric detection using ABTS in citrate buffer and plate reading at 415 nm.

### Cloning of Antibody Variable Chains

Heavy and light chain variable regions amplified essentially as described before (Wang et al., 2000). Briefly, total RNA was extracted using a kit (RNAeasy, Qiagen) and first strand cDNA generated by reverse transcription using a mixture of random hexamers and oligo(dT)20 primers. The variable regions of the heavy chains were amplified using a mixture of forward primers MH1 and MH2 and the reverse primer IGG1. The variable regions of the light chains were amplified using the forward primer MK and the reverse primer KC. PCR products were gel-purified and sequenced bi-directionally using primers MH1 and IGG1 (heavy chain PCR products) or MK and KC (light chain PCR products). Sequences were assembled and analyzed using IgBLAST (http://www.ncbi.nlm.nih.gov/igblast/).

### Quantification of Inhibitory Activity

96-well E-Plates were coated with VN (5 μg/ml) or FN (10 μg/ml) over night at 4° C. Plates were washed with PBS, added 0.1 ml serum free medium (DMEM, 0.1% bovine serum albumin, 25 mM Hepes pH 7.0) and transferred to real-time cell analyzer instrument (xCELLigence SP, Roche Corp.). Background impedance (cell index, CI) was measured, the plate removed from the instrument, the medium replaced with 15×10³ 293/uPAR cells suspended in 100 μl of serum free medium. The plate was returned to the instrument and the cell index recorded every three minutes. After 1.5-2 hours of incubation the plate was removed from the instrument and the wells added 10 μl of 200 nM pro-uPA or vehicle control, and the plate returned to the instrument. After another 1-1.5 hours of measurements the plate was removed again and wells added (20 μl) of antibody diluted to yield the indicated final concentrations. The plate was returned to the instrument and measurements conducted every 3 minutes for 2 hours and then every 15 minutes for 18 hours.

### Cell Lines and Flow-Cytometry (FACS)

293 Flp-In T-REx cells (Invitrogen Corp.) transfected with the indicated receptors or empty vector (mock), were harvested and sequentially stained with the monoclonal antibodies (10 μg/ml) and a fluorescein labeled secondary antibody. Fluorescence was recorded by flow-cytometry (FACSCalibur, BD Corp.) and the data analyzed using the software package FlowJo.

### Oligonucleotide Sequences

IGG1
(SEQ ID NO: 49)
5′-GGAAGATCTATAGACAGATGGGGGTGTCGTTTTGGC-3′
MH1
(SEQ ID NO: 50)
5′-CTTCCGGAATTCSARGTNMAGCTGSAGSAGTC-3′
(corresponding to 5′-CTTCCGGAATTC(G/C)A(A/G)GT
(A/T/G/C)(A/C)AGCTG(G/C)AG(G/C)AGTC-3′)
MH2
(SEQ ID NO: 51)
5′-CTTCCGGAATTCSARGTNMAGCTGSAGSAGTCWGG-3′
(corresponding to 5′-CTTCCGGAATTC(G/C)A(A/G)GT
(A/T/G/C)(A/C)AGCTG(G/C)AG(G/C)AGTC(A/T)GG-3′)
KC
(SEQ ID NO: 52)
5′-GGTGCATGCGGATACAGTTGGTGCAGCATC-3′
MK
(SEQ ID NO: 53)
5′-GGGAGCTCGAYATTGTGMTSACMCARWCTMCA-3′
(corresponding to 5′-GGGAGCTCGA(C/T)ATTGTG(A/C)T
(G/C)AC(A/C)CA(A/G)(A/T)CT(A/C)CA-3′).

The nomenclature IUPAC nomenclature is herein used for redundant nucleotide positions (see: http://www.bioinformatics.org/sms/iupac.html)

### Cell Binding

293/uPAR cells were seeded in FN-coated 96-well plates and allowed to adhere for 2 hours. The cells were then incubated with a constant concentration of Eu³⁺-labeled pro-uPA (4 nM, ^{Eu3+}uPA) in the presence/absence of increasing concentrations of the inhibitors to be tested (as indicated). Binding was allowed to occur for 2 hours at 4° C. after which the cells were washed to remove unbound reagents. The Eu³⁺-label was solubilized using Delfia enhancement solution and quantified by time-resolved fluorescence intensity measurements using an EnVision plate reader (PerkinElmer). The specific binding was calculated by subtracting the binding observed in wells that did not receive cells but otherwise treated identically.

### Xenograft Experiments

Six-week-old male Balb C nu/nu mice were obtained from Charles River. Before inoculation, PC-3 cells growing in serum-containing medium were washed with phosphate buffered saline (PBS), harvested by trypsinization, and pelleted at 1200 rpm for 7 minutes. Cells (1.0×10⁶) were resuspended in 200 μl of PBS with 20% Matrigel. Animals were anesthetized by intraperitoneal (i.p.) injection of Avertin and 1.0×10⁶ cells were inoculated subcutaneously (s.c.) using a 26-gauge needle into the right flank of anesthetized mice. 5 days after xenografting, the animals were randomized into 2 control groups, where animals were treated twice a week i.p. with vehicle (n=5, PBS), non-immune mouse IgG1 (n=5, 10 mg/kg), and two experimental groups where animals were treated with either mAb 8B 12 (n=5, 10 mg/kg) or mAb 13F6 (n=5, 10 mg/kg). The animals were monitored twice a week for 7 weeks for tumor development and growth. Tumor volume was determined according to the formula: tumor volume=shorter diameter²×longer diameter/2. One mouse that did not develop palpable tumors, (one from the IgG control group) was excluded from the data analysis. There was no significant difference between tumor growth in PBS and IgG1 treated animals (data not shown) and the data from these mice were pooled (n=9) for the comparison with the experimental 8B12 (n=5) and 13F6 (n=5) groups. Results were analyzed as the mean±SE, and comparisons of the experimental data were analyzed by unpaired, two-tailed, equal variance, t-test.

### Immunohistochemical Analyses

For immunohistochemical analysis, primary tumors were excised, fixed in 4% paraformaldehyde (Formalin) and embedded in optimal cutting temperature (OCT) resin (Killik, BIO-OPTICA). Tissue blocks were sectioned at 8 μm and mounted onto positively charged glass slides for immuno-staining. For Ki-67 staining, sections were incubated with acetone at 4° C. for 1 minute. Slides were washed with PBS followed by blocking in pre-incubation buffer (PBS with 6% BSA and 10% FBS) for 1 h at RT. Slides were incubated with Ki-67 antibody (diluted 1:500) overnight at 4° C. followed by washing with PBS. For detection, anti-rabbit Cy3 (1:200) and DAPI (1:2500) were used. Slides were mounted with Vectamount AQ. For detection of apoptotic cells, sections were incubated with 80% ethanol at room temperature for 1 minute. Slides were washed with PBS followed by blocking in pre-incubation buffer for 1 h at RT. Primary antibody (Cleaved caspase-3, 1:200) incubation was done overnight at 4° C. followed by washing with PBS. Detection was done as for Ki-67 stained slides. For quantification of cell proliferation and apoptosis, a total of 24 sections per animal were analyzed at 10× magnification, respectively. Data are shown as the average number of positive cells per field.

### Results

### Background and Rationale

As described in Example 1 the uPA/uPAR-chimeras ^{GFD}uPAR-hFc, ^{GFD}uPAR-mFc and ^{GFD}uPARmyc display a dramatically increased (>10.000-fold) binding affinity for VN as compared to “conventional” forms of soluble uPAR. It is plausible that this increased binding is caused by a more efficient exposure of the VN binding site in these chimeras. The presence of an efficiently exposed VN binding site suggests that these chimeric receptor can be exploited for the generation and/or isolation of molecules that bind to the VN binding site in uPAR and have competitive antagonistic activity. In fact, in this example authors show that monoclonal antibodies raised against ^{GFP}uPAR-hFc frequently bind to the VN binding site in uPAR and often are potent inhibitors of uPAR function.

### Immunization

To generate monoclonal antibodies against ^{GFD}uPAR-hFc, three C57Bl6 uPAR^{-/-} animals were immunized with recombinant ^{GFD}uPAR-hFc according to well-established procedures (see materials and methods). All mice were initially immunized with ^{GFD}uPAR-hFc and received three post-immunization boosts with the same antigen. After a seven-week rest period, one animal (Ms^{#}21574) received a pre-fusion boost with ^{GFD}uPAR-hFc and spleens were removed four days later and splenocytes fused to the mouse SP2/0 myeloma cell line by the polyethylene glycol method using standard procedures (Galfre et al., 1977). The remaining two mice (Ms^{#}1416 and Ms^{#}1417) were boosted other three times with reduced amounts of ^{GFD}uPAR-hFc and after a seven-week rest-period given a pre-fusion boost with ^{GFD}uPAR-mFc, splenocytes were isolated and fused to the mouse SP2/0 myeloma as above. The additional boosts with reduced levels of antigen were done in an attempt to raise the affinity of the resulting antibodies. The final boost with ^{GFD}uPAR-mFc was done to reduce the number of antibodies reactive with the hFc portion of ^{GFD}uPAR-hFc as about half of the positive hybrids identified after the first fusion were found to recognize hFc (data not shown).

### Identification of Positive Hybrids, Sub-Cloning and Isotyping

The three different fusions yielded a total of 17 (12, 3 and 2 from Ms^{#}21574, Ms^{#}1416 and Ms^{#}1417, respectively) hybrids that grew and displayed continuous production of immunoglobulin reactive with ^{GFD}uPAR-hFc and negative for binding to hFc. Of the obtained hybrids, 8 (5, 2 and 1 from Ms^{#}21574, Ms^{#}1416 and Ms^{#}1417, respectively) were subcloned by limited dilution to ensure clonality. Immunoglobulin was purified from the conditioned medium by standard Protein A affinity chromatography and the isotypes determined using a commercial kit. The mouse ID, clone and subclone number and immunoglobulin isotype is shown in Table 1. To determine the sequence of the variable regions, RNA was extracted from growing hybridoma culture, reverse transcribed, amplified and sequenced. In FIG. 9, the deduced amino acid sequence of the heavy and light chain variable regions are shown numbered according to the Kabat system with the complementarity determining regions (CDRs) underlined.

Reactivity of Antibodies with Cell Surface uPAR

As the prime use of the antibodies is as inhibitory reagents binding to cell-surface uPAR, authors first tested the specificity of the antibodies by flow cytometry on 293 cells transfected with empty vector or expressing human uPAR (huPAR) or mouse uPAR (muPAR). As shown in FIG. 10, all 8 monoclonal antibodies (mAb) (first and second row of histograms) bind specifically and efficiently to cells expressing human uPAR. With the exception of one antibody (19.10), all the antibodies display pronounced species selectivity for human uPAR as they fail to label cells expressing mouse uPAR efficiently. The only exception, 19.10, displays partial reactivity with mouse uPAR. Two mAbs specific for mouse uPAR (BR4 and AK17, (Tjwa et al., 2009)) were included in the analysis as controls.

### Inhibitory Activity

To evaluate the activity of the different antibodies in inhibiting uPAR-signaling in live cells, authors quantified cell adhesion by impedance measurements using a real-time cell analyzer (RTCA, xCELLigence SP, Roche Corp.) (FIG. 11). In these experiments, authors utilized 293 cells expressing uPAR (293/uPAR) as these display strong uPAR-dependent cell adhesion to VN (Madsen et al., 2007). When 293/uPAR cells are seeded in VN or fibronectin (FN) coated wells, the cells adhere and spread on the substrate resulting in a time-dependent increase in cell index. After approximately 1.5-2 hours of cell adhesion, cells are either treated with vehicle control (FIG. 11A) or pro-uPA (FIG. 11B). The treatment with pro-uPA saturates uPAR with ligand and enhances uPAR-dependent cell adhesion to VN (Madsen et al., 2007) as documented here by the robust increase in cell index observed after pro-uPA addition (compare black curves in FIG. 11 panels A and B and note the fast increase in cell index upon pro-uPA addition). Treatment with pro-uPA does not modulate cell adhesion to FN, which is mediated by integrins (Madsen et al., 2007), and consistently the treatment with pro-uPA does not enhance the cell index in FN coated wells noticeably (FIG. 11C). Approximately one hour after pro-uPA (or vehicle) treatment, diluted amounts of antibody were added and the changes in cell index recorded over time. Inhibitory activity was quantified as the reduction in cell index observed one hour after addition of the antibody relative to vehicle treated cells and IC50 values calculated by non-linear regression as illustrated in FIG. 11D. The data shown in FIG. 11 show the analysis of one antibody (8B12) and a summary of the data obtained for all the antibodies can be found in Table 2. Of the eight antibodies characterized in this example, six (8B12, 10H6, 13D11, 19.10, AL38 and BE18) were found to inhibit basal uPAR-mediated cell adhesion to VN with IC₅₀ values in the low nanomolar range. In the presence of pro-uPA, four of the six antibodies (8B12, 10H6, 13D11, 19.10) retained roughly unaltered inhibitory activity while two (AL38 and BE18) were found to be non-inhibitory under these conditions. The ability to inhibit uPAR-mediated cell adhesion to VN in the presence of pro-uPA is a unique feature of 8B12, 10H6, 13D11 and 19.10 as the known inhibitory antibodies (R3 and R5) were found to be inactive under these conditions (see below). The inhibitory activity of the antibodies was highly specific for cell adhesion to VN, as they did not modulate cell adhesion to FN (FIG. 11 panels C and D and data not shown).

Comparative Analysis of the Inhibitory Activity of 8B12 with Other Known Inhibitors of the uPAR/VN-Interaction and/or uPAR Function

Various inhibitors of the non-proteolytic activities of uPAR have been described. These include the uPAR-binding N-terminal domain of VN (the Somatomedin B domain, SMB) that represents the natural competitive antagonist of the uPAR/VN-interaction (Deng et al., 1996), a synthetic peptide (P7) isolated by phage display and shown to interfere with VN-binding to uPAR (WO97/35969), two well-described conventional antibodies (R3 and R5) known to interfere with the uPAR/VN-interaction and uPAR-function (Sidenius and Blasi, 2000), and the ATN-658 antibody (WO 2008/073312; WO2005/116077) that has been shown to reduce tumor volume and skeletal lesions in a model of prostrate cancer (Rabbani et al., 2010), reduce small-volume and established disease in a model of colorectal cancer cell growth in the liver (Van Buren et al., 2009) and to reduce ovarian cancer metastasis (Kenny et al., 2010). The location of the minimal ATN-658 binding epitope in uPAR (268KSGCNHPDLD277, Seq ID no. 16 in WO 2008/073312, corresponding to aa. 268-277 of SEQ ID NO:1) is close to the C-terminal of uPAR and distinct from the epitope bound by the inhibitory antibodies described in this invention (R89, R91 and Y92). As a surrogate for ATN-658, authors used the well-described R2 antibody that binds uPAR in the exact same epitope as ATN-658 (residue D275 in the 268KSGCNHPDLD277 sequence, corresponding to aa. 275 of SEQ ID NO:1, is critical for R2 binding to uPAR (Gardsvoll et al., 2007)).

To compare the function inhibitory activity of the above-mentioned compounds, authors analyzed these in the same assay applied to determine the inhibitory activity of the antibodies described in this invention (see FIG. 11). The results of these analyses are graphically presented in FIG. 28 and numerically in Table 2.

In terms of IC50 values, quantified in the absence of pro-uPA, the 8B12 antibody is 4-7 fold more potent than the R3 and R5 antibodies, 34-fold more potent than the R2 antibody, 260-fold more potent than the SMB domain and more than 2000-fold more potent than the P7 peptide. Note that the maximal inhibition attained with all of these compounds (with the exception of the SMB-domain) are inferior to that attained with 8B 12 suggesting that the purely IC50-based comparison used here actually under-estimates the inhibitory activity of 8B12.

When quantified in the presence of pro-uPA, only the SMB domain and the R2 antibody were found to be significantly (>20%) inhibitory. In terms of IC50 values, 8B12 was found to be 25-fold more potent than R2 and 330-fold more potent than the SMB-domain. Given the extremely poor activity of the P7 peptide measured in the absence of pro-uPA, this compound was not tested in the presence of pro-uPA. Note that in addition to the 25-fold difference in IC50 between 8B12 and R2, the latter also display about 4-fold reduced maximal inhibition suggesting that also in the presence of pro-uPA the inhibitory activity of 8B12 is under-estimated. As R2 and ATN-658 bind to the same epitope in uPAR it is plausible that the activity of 8B12 is similarly superior to this antibody, which has confirmed in vivo efficacy.

Mapping of the Binding Epitopes in uPAR

To determine the molecular basis for the inhibitory activity of the antibodies, authors next aimed at mapping their binding epitopes in uPAR. Authors have previously described a complete functional alanine scan (the systematic substitution of individual residues with alanine) of uPAR in cell culture (Madsen et al., 2007). Detergent lysates of cells expressing the 255 different uPAR-mutants are available to the authors that therefore conducted ELISA assays to identify uPAR-mutants displaying reduced reactivity with the different antibodies. For a number of reasons, the data-quality of this screen was not sufficiently high to determine unequivocally the reactivity of the different monoclonal antibodies with the different uPAR alanine substitution mutants. Nevertheless, more than one of the inhibitory antibodies seemed to display reduced reactivity with uPAR-mutants having alanine substitution in the region close to R91 (data not shown). To investigate this finding in a more rigorous manner, authors conducted flow cytometry analysis (FACS) on 293 cells expressing selected uPAR alanine substitution mutants in this region (FIGS. 12, 13 and 14). Authors first compared the reactivity of the different antibodies with wild-type uPAR and a double alanine substitution mutant of R83 and R89 (FIG. 12). As shown, five of the eight antibodies (8B12, 10H6, 13D11, 19.10 and AL6) displayed strongly reduced reactivity with the R83/89A mutant suggesting that the binding epitope for these antibodies includes R83 and/or R89. The reason for the reduction in staining intensity is not a result of a lower expression level of this receptor mutant as the three remaining antibodies (13F6, AL38 and BE18) stained wild type and mutant receptor equally well. In the same experiment, authors also addressed the effect of pro-uPA occupancy of the receptor on antibody recognition. The binding of most antibodies, including all of the inhibitory antibodies, was not notably affected by the presence of uPA, demonstrating that the binding sites for pro-uPA and these antibodies are non-overlapping. In contrast, the antibodies AL38 and BE18 displayed strongly reduced reactivity in the presence of pro-uPA, suggesting that these antibodies recognize epitopes overlapping with the uPA binding-site in uPAR. To determine if the reduction in recognition of the R83/89A receptor was due to the R83A and/or R89A mutation, authors next analyzed cells expressing uPAR mutants carrying discrete R83A and R89A substitutions as well as a alanine substitution of another arginine residue in this region (R91A) of uPAR and known to be important for VN binding to uPAR (Gardsvoll and Ploug, 2007; Madsen et al., 2007) (FIG. 13). As it can be seen, the result of this experiment clearly shows that R91 and R89, but not R83, are part of the recognition epitope for the inhibitory antibodies 8B12, 10H6, 19.10 and 13D11 as well as for the non-inhibitory antibody AL6. The binding of these antibodies to uPAR is virtually abrogated by the R91A mutation, strongly impaired by the R89A mutation and unaffected by the R83A substitution. Cells expressing R83A, R89A and R91A mutant receptors were stained equally well by the remaining antibodies (13F6, AL38 and BE18) documenting that the epitopes recognized by these antibodies lie outside of this region and that the mutant receptors are expressed equally well. To complete the analysis of this region of uPAR, authors analyzed another set of uPAR mutants (S88A, S90A and Y92A) as well as a distant mutation (P218A) and a deletion mutant of uPAR where residues 1-83 (domain D1) (corresponding to aa. 1-83 of SEQ ID NO:1) have been deleted (i.e. residues 84 to 283 are retained—domains D2 and D3) (FIG. 14). As it can be seen from the data, the result of this analysis demonstrates that in addition to R91 and R89 described above, also Y92 is important for binding of the inhibitory antibodies to uPAR. All antibodies recognize the truncated version of uPAR lacking D1 (D2D3 see FIG. 15) less well than the full-length receptor suggesting that this receptor is expressed at lower levels on the cell surface. One antibody (13F6) recognizes the D2D3 receptor better than the other antibodies, whereas remaining antibodies recognize D2D3 less well than the intact receptor.

The 8B12 Antibody is a Specific Inhibitor of the VN-Dependent Functions of uPAR and does not Interfere with the Proteolytic Functions of the Receptor Dependent on uPA-Binding.

The activity of 8B12 in inhibiting the uPAR-dependent cell adhesion to VN is intact even in the presence of uPA (see FIG. 11) suggesting that this antibody is a specific inhibitor of the VN-dependent uPAR functions. This is consistent with its binding epitope of this antibody being centered on the VN-binding site in uPAR (R91) (see FIGS. 13, 14 and 15) that is not involved in uPA-binding. To experimentally determine if 8B12 interferes with uPA-binding to uPAR, and thus with the proteolytic functions of the receptor, authors conducted binding assays in which uPAR-expressing 293 cells (293/uPAR) were incubated with a fixed concentration of Europium-labeled pro-uPA (^{Eu3+}uPA) together with increasing concentrations of the compound to be tested. As shown in FIG. 16, the antibodies 8B12 and 13F6 display no or minimal competitive activity in this assay while the control antibody R3 and un-labeled pro-uPA (self-competition) efficiently inhibited binding of ^{Eu3+}uPA to 293/uPAR cells. These data document that 8B12 does not interfere with uPA binding to uPAR and thus demonstrate that this antibody is a selective inhibitor of the VN-dependent uPAR-function. This renders 8B12, and the other inhibitory antibodies described here (10H6, 13D11 and 19.10), unique. R3 and similar antibodies interfere with both uPA and VN binding to uPAR (see FIG. 16 and FIG. 28).

### The 8B12 Antibody Reduces Tumor Growth in a Xenograft Model of Prostate Cancer (PC3)

To determine the potential anti-tumor activity of mAb 8B12 in vivo, we conducted studies using a prostate cancer xenograft model. In this model, one million PC3 cells were inoculated in the right flank of male Balb C nu/nu mice through subcutaneous route. The xenografted animals were treated bi-weekly with mAb 8B12, the non-inhibitory mAb 13F6, a control mouse IgG or PBS (vehicle) by intraperitoneal injections and the volume of the tumors monitored by calibration. As shown in FIG. 17, the animals treated with mAb 8B12 displayed significantly reduced tumor volumes as compared to control animals. Treated animals displayed a 30-40% reduction in tumor volume, which is comparable to that observed by others using an inhibitory anti-uPAR antibody ATN-658 (Rabbani S A, et al. Neoplasia 2010). A similar inhibition was not observed for the non-inhibitory antibody 13F6 demonstrating that the mechanism behind the inhibitory activity of 8B12 is its inhibition of VN-binding and not merely targeting of uPAR-expressing cells.

Treatment with 8B12 Antibody Reduces Cell Proliferation and Increase Apoptosis in Xenografted PC3 Tumors

To investigate the biological reason for the reduced PC-3 tumor growth in animals treated with 8B12, authors conducted immunohistochemistry analysis of sections of tumors taken from animals 8 weeks after xenografting (FIG. 18). To evaluate tumor cell proliferation, authors stained for the proliferating cell antigen Ki-67 and to evaluate apoptosis they stained for activated (cleaved) Caspase-3. As illustrated in FIG. 18A and quantified in FIG. 18B, tumors taken from mice treated with 8B12 display a strong increase in the number of cells undergoing apoptosis as evidenced by cleaved Caspase-3 reactivity and a marked decrease in the number of proliferating cells as marked by Ki-67 positivity suggesting that mAb 8B12 suppresses tumor growth by promoting apoptosis and by reducing cell proliferation. Treatment with the non-inhibitory mAb 13F6 antibody did not cause any significant changes in cell proliferation and apoptosis supporting that it is not the simple targeting of uPAR expressing cells that is responsible for the biological activity of 8B12, but rather that the inhibitory action on the uPAR/VN-interaction is required.

### Conclusions

In this example, authors have shown that antibodies raised against ^{GFD}uPAR-hFc frequently are functional inhibitors of uPAR. The more potent inhibitory antibodies identified herein (8B12, 10H6, 19.10 and 13D11) all bind uPAR in the same region, the critical residues being R91, R89 and Y92 (FIG. 15). The binding site of the antibodies coincides partially with the published physical (Huai et al., 2008) and functional (Madsen et al., 2007) binding site for VN in the receptor demonstrating that functional inhibitory activity of these antibodies is mediated by competitive antagonism of the uPAR/VN-interaction. These data furthermore document that the VN binding site in uPAR is exposed in ^{GFD}uPAR-hFc and that this region is antigenic in mice. The authors have shown that 8B12 is a selective inhibitor of the VN-dependent uPAR functions. Furthermore, 8B12 inhibits tumor growth by reducing tumor cell proliferation and increasing apoptosis.

### EXAMPLE 3

### Materials and Methods

Cloning of ^{mGFD}muPAR-Fc

The expression vector encoding ^{mGFD}muPAR-Fc was generated by amplification of a mouse uPA cDNA with oligos muPAkf/mGFDr and a mouse uPAR cDNA with oligos muL8f/MUPPFCR. The two PCR products were mixed, co-amplified with oligos muPAkf/MUPPFCR and cloned KpnI/XhoI in the vector pFRT/TO-Fc. The protein encoded by this vector (^{mGFD}muPAR-Fc, Sequence 9 (SEQ ID NO: 17)) is composed of the 49 N-terminal residues of mouse uPA including the growth factor domain (GFD, Sequence 9A (SEQ ID NO: 4)), a short linker (amino acids GGAGAAGG, Sequence 9B (SEQ ID NO: 8)), residues 1-273 of mouse uPAR (Sequence 9C corresponding to aa. 1-273 of SEQ ID NO: 2), a second short linker (amino acids VELEVLFQGPIE, Sequence 9D (SEQ ID NO: 11)) and a human Fc-tag (Sequence 1C (SEQ ID NO: 5)).

### Oligonucleotide Sequences

muPAkf:
(SEQ ID NO: 54)
5′-GGGGTACCATGAAAGTCTGGCTGGCGAG-3′
mGFDr:
(SEQ ID NO: 55)
5′-CGCCCCGGCCCCTCCTTTTGATGCATCTATCTCACA-3′
muL8f:
(SEQ ID NO: 56)
5′-GGAGGGGCCGGGGCGGCTGGAGGACTGCAGTGCATGCAGTGTGAG-3′
MUPPFCR:
(SEQ ID NO: 57)
5′-AGCGGCTGTAACAGCCCCGTCGACCG-3′

### Generation of Antibodies

Monoclonal antibodies against ^{mGFD}muPAR-Fc were raised in uPAR^{-/-} mice as described for human ^{GFD}uPAR-hFc variant (see Example 2, section Materials and Methods).

### Cell Binding Assay

30×10³ 293 cells expressing human uPAR (293/uPAR) suspended in DMEM containing 0.1% BSA and 25 mM Hepes pH 7.0 (binding buffer) were seeded in fibronectin coated (10 μg/ml in PBS) black 96-well ELISA plate (NUNC) wells and allowed to adhere for 2-4 hours at 37° C. After gentle washing, cells were incubated with a fixed concentration (4 nM) of Eurobium-labeled pro-uPA (^{Eu+}uPA) in the presence or absence of the competitors to be tested. Binding was allowed to occur for 1 hour at 4° C. and unbound reagents gently removed by repeated washings using cold binding buffer. The cells were lysed by addition of 0.1 ml Enhancement Solution (Perkin Elmer) and the Eu³⁺label quantified by time-resolved fluorescence intensity measurement (Delfia, Perkin Elmer) using a EnVision plate reader (Perkin Elmer). Specific binding was calculated by subtracting the binding measured in wells receiving no cells, but otherwise treated in the same way.

### Results

### Background and Rationale

None of the inhibitory antibodies described above bind to mouse uPAR (see FIG. 10) and are therefore unlikely to have any effect on uPAR-expressing cells of the host in rodent xenograft models of human cancer. The efficacy of mAb 812 in reducing tumor growth thus shows that the antibody is likely to be acting directly on the xenografted human cancer cells. Nevertheless, the species specificity of these antibodies impedes reliable pre-clinical testing because the possible positive or negative effects on host cells cannot be addressed. To bypass this limitation, authors set out to generate antibodies with similar inhibitory activity, but effective also on mouse uPAR.

Construction and Production of a Murine uPAR (^{mGFD}muPAR-hFc) Displaying Constitutive Active VN-Binding

With this aim, authors constructed a constitutively active mouse uPAR (^{mGFD}muPAR-hFc, FIG. 19A) essentially as described for GFDuPAR-hFc, but assembled using the mouse-derived sequences encoding GFD and uPAR. As predicted the resulting chimera binds with high affinity to immobilized VN (Kd=0.82 nM, FIG. 19B), demonstrating that this strategy is versatile and applicable to GFD-domains and uPAR's of different species origin.

Antibodies Raised Against ^{mGFD}muPAR-hFc are Potent Inhibitors of Mouse uPAR Mediated Cell Adhesion to VAT

To generate monoclonal antibodies against ^{mGFD}muPAR-hFc, five C57Bl6 uPAR−/− animals were immunized with recombinant ^{mGFD}muPAR-hFc as described above for the human GFDuPAR-hFc. Spleens from the two best responding animals were removed and splenocytes fused to the mouse SP2/0 myeloma cell line by the polyethylene glycol method using standard procedures.

The two fusions yielded a total of 13 hybrids that grew and displayed continuous production of immunoglobulin reactive with ^{mGFD}muPAR-hFc and negative for binding to the Fc tag (data not shown). To identify those hybrids producing inhibitory antibody, the conditioned medium were tested in cell adhesion assays to VN using 293 cells expressing mouse uPAR (FIG. 20). In this assay, the supernatant of four different hybrids (OOF12, NM23, NE43 and OMD4) displayed evident inhibitory activity.

The inhibitory antibodies raised against constitutively active human uPAR (GFDuPAR-hFc) were found to recognize an epitope in human uPAR coinciding with the VN binding-site including the functionally important Arg91 (R91). To determine if the same remarkable specificity is also observed for the inhibitory antibodies raised against constitutively active mouse uPAR (^{mGFD}muPAR-hFc), authors tested the reactivity of the antibodies in the hybridoma supernatants with immobilized ^{mGFD}muPAR-Fc and a single point mutant of this receptor in which Arg 92 (R92) (corresponding to Arg91, R91, in human GFD uPAR) had been substituted with an alanine residue (^{mGFD}muPAR-Fc R92A). The supernatants of two of the hybridomas (OMD4 and NE43) displayed a clear preferential binding to non-substituted ^{mGFD}muPAR-Fc (FIG. 21). As the supernatants of these two hybrids were also found to be inhibitory, this suggests that the produced antibodies are competitive inhibitors of the VN/muPAR interaction through binding to the VN binding site in muPAR. The other two inhibitory hybrids (OOF12 and NM23) recognized substituted and non-substituted ^{mGFD}muPAR-Fc equally well suggesting that their inhibitory activity is mediated through binding to different epitopes. In this assay, they also tested the reactivity with human soluble uPAR (suPAR) to determine if the generated antibodies cross-react with human uPAR. One antibody (OMD4) was found to display reactivity with human uPAR.

The four hybrids displaying inhibitory activity were selected for further analysis and subcloned by limited dilution to ensure clonality. Immunoglobulin was purified from the conditioned medium by standard Protein A affinity chromatography and the isotypes determined as described in Example 2. The mouse ID, clone and subclone number and immunoglobulin isotype are shown in Table 4. To determine the sequences of the variable regions, RNA was extracted from growing hybridoma culture, reverse transcribed, amplified and sequenced. In FIG. 22 the deduced amino acid sequence of the heavy chain variable regions are shown numbered according to the Kabat system with the complementarity determining regions (CDRs) underlined.

Finally authors tested and compared the species-specificity of the generated antibodies with that of mAb 8B12 raised against GFDuPAR-hFc (FIG. 23) and 13F6. Consistent with its inhibitory activity (FIG. 20), the binding epitope dependence on R92 (FIG. 21) and the reactivity with human uPAR (FIG. 21), the antibody OMD4 was found to inhibit cell adhesion mediated by both human and mouse uPAR. All the other generated antibodies were found to display species specific inhibition with NM23, OOF12 and NE43 being highly selective inhibitors of mouse uPAR. The 13F6 antibody was included as a negative control reactive with human uPAR.

### Conclusions

Authors have shown that constitutively active mouse uPAR variants can be readily generated and that these can be used to generate inhibitory antibodies with a high frequency (4 out of 13); the inhibitory activity of these antibodies is frequently mediated by direct binding of the antibody to the VN binding site (2 of 4).

Moreover, the use of constitutively active mouse uPAR as antigen allows for the generation of inhibitory antibodies reactive specifically with the mouse receptor (OOF12, NM23 and NE43) as well as antibodies reactive with both the mouse and human receptor (OMD4). The latter antibodies will greatly facilitate future pre-clinical studies in mouse models.

### EXAMPLE 4

### Materials and Methods

### Antigen Preparation

The expression and purification of uPAR-hFc is described in Example 1. VN(1-66)-Fc has been described previously (Madsen et al.). Pro-uPA was a kind gift of Jack Henkin, Abbot laboratories.

### Panning Procedure

For each round of panning, three NUNC immunotubes (A, B and C) were prepared and incubated as described below. All incubations were conducted in a total volume of 4 ml and coatings were done in 50 mM carbonate buffer, pH 9.6. Tubes were first coated overnight with anti-human Fc antibody (tube A: 150 μg/ml and tube C: 15 μg/ml) or pro-uPA (tube B: 150 μg/ml). The tubes were washed 3 times with PBS, blocked for 2 hours in PBS containing 2% milk (2% MPBS), and incubated with VN(1-66)-Fc (tube A: 150 μg/ml) and tube C with a mixture uPAR/Fc (15 μg/ml) and pro-uPA (15 μg/ml). Tubes were washed with PBS to remove unbound reagents and kept in 2% MPBS until use. In the first panning step (negative selection), 10¹³ t.u. (titration unit) of phage-library diluted in 4% MPBS was added to tube A and incubated for 2 hours at RT with 30 min of repeated inversion followed by 1.5 hr in upright position. In the second (negative) selection step, the supernatant of tube A was transferred to tube B and incubated as above. For the third (positive selection) panning step, the supernatant of tube B was transferred to tube C and incubated as above. At the end of the incubation, the supernatant was discarded and the tube washed 10 times with PBS containing 0.1% Tween-20 and 10 times with PBS to remove weakly bound phages. Bound phages were eluted using 1 ml of 100 mM triethylamine for 5 min at RT with repeated inversion. The phage eluate was neutralized with 0.5 ml of 1M Tris HCl, pH 7.4 and used to infect 10 ml of growing TG1 culture (OD=0.4). Infected TG1 cells were spread onto large selection plates, grown overnight at 30° C., and harvested by scraping. Phages were amplified by VCS M13 (Stratagene Corp.) helper phage infection in liquid culture. Phages were harvested from the culture supernatant and concentrated by PEG precipitation. The 2nd and 3rd rounds of panning were conducted like the 1st round with the only exception that the concentrations of bait proteins used in the negative selection steps (i.e. anti human Fc antibody, VN(1-66)-Fc and pro-uPA) were all to 15 μg/ml.

### Results

### Background and Rationale

In Examples 1, 2 and 3, authors have shown that engineered forms of uPAR displaying high VN-binding activity can be applied for the generation of natural antibodies that are strong inhibitors of uPAR function. The antibodies generated in Example 2 and 3 are murine and may thus be immunogenic in humans possibly limiting clinical use. Several ways have been developed to generate fully human antibodies and authors here exploit phage display to isolate human single chain variable fragments (scFv) antibodies that specifically interact with uPAR and inhibit its function. As the complex between uPAR-hFc and pro-uPA displays high VN-binding activity (see FIG. 2A), authors reasoned that isolation of phages binding to this complex would enrich for phages binding to the VN-binding site in uPAR as illustrated in the cartoon in FIG. 24.

Phage Display scFv Library

The synthetic human antibody phage display library applied here (ETH-2-Gold) has been described previously (Silacci et al., 2005) and has a complexity of 3 billion unique sequences. The library is available from Philogen (http://www.philogen.com/) and newer libraries with even higher complexity are now available. The phage library was handled and screened in close accordance with the detailed protocols available on the Internet at URL http://www.pharma.ethz.ch/institute_groups/biomacromolecules/protocols/eth

### Selection Procedure

To isolate scFv-antibodies binding to ligand-occupied dimeric uPAR, authors employed a panning strategy based on repeated rounds of negative and positive selection to enrich for phages binding to uPAR-hFc occupied by pro-uPA and to eliminate phages binding to the non-uPAR components of the positive enrichments step. The panning procedure is illustrated in FIG. 24. In a first negative selection step, phages binding to human Fc (hFc), the goat anti-human Fc-antibody (anti-hFc) used for capture and pro-uPA are removed by adsorption of suspended phages to immobilized hFc, anti-hFc and pro-uPA. Non-adsorbed phages are transferred to the second positive selection step in which the complex between uPAR-hFc and pro-uPA bound to immobilized anti-hFc antibody was used to capture phages.

### Identification of Positive Clones

A total of 564 clones were picked after 2 and 3 rounds of selection and small-scale scFv production induced by IPTG addition to liquid cultures grown in 96-well plates. The bacterial supernatants were assayed by ELISA for the presence of scFv binding activity towards the proteins and protein complexes used in the positive and negative panning steps. Of the analyzed supernatants, 59% (n=335) scored positive (ELISA signal greater than 3-fold over background) for binding to the positive bait. Of these, only 12% (n=41) scored positive also with the negative bait. These data demonstrate that the negative selection procedure is effective in removing phages reactive with non-desired components of the protein complex used for the positive selection.

### Sequence Analysis of Positive Clones

Plasmid DNA was isolated only from clones reactive with the positive bait and subjected to sequencing. Of these, 225 clones yielded high-quality sequence information of the heavy and light chain complement determining 3 regions (HC-CDR3 and LC-CDR3) and the analysis was restricted to these. A total of 13 unique sequences were found with a single sequence accounting for about 90% (n=200) of all the clones. Manual inspection for evident sequence homology suggests that the 13 unique sequences can be grouped into 6 different classes (A-F) assumed to have similar binding specificity (Table 3). One representative clone of each of the 13 unique sequences was selected and scFv expressed and purified according to standard protocols.

scFv's Isolated Using the Pro-uPA:uPAR-hFc Complex Bind Cell Surface uPAR and their Binding is Modulated by Pro-uPA.

To determine the specificity of the scFv, authors conducted FACS analysis on 293 cells expressing human uPAR and the histogram data shown in FIG. 25 and summarized in Table 3. Four clones (1G5, 3D9, 2H10 and 3C10) gave strong positive staining, two clones gave intermediate staining (1C1 and 3B6), and two clones only weak staining (2G5 and 105). The remaining 7 clones were negative (data not shown) and not further analyzed. The staining was conducted in the presence or absence of pro-uPA to determine if the exposure of the scFv binding epitopes was modulated by ligand occupancy. In one clone (3B6) the presence of pro-uPA increased the staining intensity and in three clones (1C1, 3C10 and 2G5) it reduced it.

scFv's Isolated Using the Pro-uPA:uPAR-hFc Complex Inhibit uPAR Function

Initial testing showed that three scFv's (1C1, 3B6 and 3C10) inhibited uPAR mediated 293 cell adhesion to VN (data not shown), however, because of difficulties in expression and purification of scFv 1C1, only 3B6 (which is very similar to 1C1 in sequence) and 3C10 were analyzed in more detail. To quantify the inhibitory activity, authors conducted real time cell assays exactly as described for the monoclonal antibodies in Example 2. As shown in FIG. 26A, 3B6 inhibits uPAR mediated cell adhesion to VN in a dose-dependent manner Also in the presence of pro-uPA (FIG. 26B), the scFv 3B6 reduced cell adhesion, however, with reduced efficiency. 3B6 did not affect cell adhesion to FN documenting its specificity (FIG. 26C). By non-linear regression analysis of dose response curves (FIG. 26D), the IC₅₀ concentrations were calculated to be 561 nM and 2220 nM in the absence and presence of pro-uPA, respectively. Similarly, scFv 3C10 inhibited uPAR-mediated cell adhesion in the absence of pro-uPA in a dose-dependent manner (FIG. 27A). This scFv was however without notable activity when assayed in the presence of pro-uPA (FIG. 27B). Again, the inhibitory activity was specific for uPAR-mediated cell adhesion to VN, as it had no effect on adhesion to FN (FIG. 27C). From non-linear regression analysis of the dose response curve (FIG. 27D), the IC₅₀ concentration in the absence of pro-uPA was calculated to be 108 nM.

### Tables

TABLE 1
Monoclonal antibodies

| Mouse | Clone | Subclone | Isotype |
|---|---|---|---|
| #21574 | 13F6 | 13F6.1.4 | IgG1 κ |
| #21574 | 8B12 | 8B12.3 | IgG1 κ |
| #21574 | 10H6 | 10H6.3.76.1 | IgG1 κ |
| #21574 | 13D11 | 13D11.78.26 | IgG1 κ |
| #21574 | 19.10 | 19.10.3 | IgG2b κ |
| #1416 | AL6 | AL6.1.1 | IgG2b κ |
| #1416 | AL38 | AL38.27 | IgG1 κ |
| #1417 | BE18 | BE18.4.2 | IgG1 κ |

Table indicating the mouse ID, clone number, subclone number, and the isotype of the monoclonal antibodies described in this example.

TABLE 2
Inhibitory activity of monoclonal antibodies and activity
comparison with other published inhibitors of the uPAR/
VN-interaction and/or of uPAR-function

| Anti- | % max | | | | | |
|---|---|---|---|---|---|---|
| body/ | inhi- | IC₅₀ (veh. | | % max | IC₅₀ (pro-uPA | |
| reagent | bition | pre-treat.) | | inhibition | pre-treat.) | |
| 13F6 | <20 | non inhibitory | | <20 | non inhibitory | |
| 8B12 | 90 | 1.8 | (1.4-2.3) | 89 | 2.4 | (2.0-2.8) |
| 10H6 | 39 | 4.0 | (3.4-4.7) | 78 | 3.7 | (3.2-4.2) |
| 13D11 | 38 | 3.7 | (2.8-4.9) | 82 | 4.9 | (4.7-5.3) |
| 19.10 | 67 | 1.9 | (1.4-2.4) | 79 | 3.6 | (3.4-3.9) |
| AL6 | <20 | non inhibitory | | <20 | non inhibitory | |
| AL38 | 68 | 2.4 | (1.8-3.2) | <20 | non inhibitory | |
| BE18 | 37 | 5.8 | (3.2-10.4) | <20 | non inhibitory | |
| R2 | 65 | 62 | (47-82) | 21 | 104 | (47-235) |
| R3 | 53 | 8.2 | (7.5_9.0)) | <20 | non inhibitory | |
| R5 | 56 | 14 | (12-18) | <20 | non inhibitory | |
| SMB | 100* | 469 | (387-569) | 100* | 800 | (745-859) |

| | | | | | |
|---|---|---|---|---|---|
| P7 | 38 | 4683 | (1465-14983) | Not tested | Not tested |

Summary of the inhibitory activity of different monoclonal antibodies. “Non-inhibitory” indicates that less than 20% inhibition was observed at the highest tested antibody concentration (300 nM). The IC₅₀ values (i.e. the concentration required to attain half-maximal inhibition) and their associated 95% confidence intervals (indicated in parentheses) were determined as shown for antibody 8B12 in FIG. 11. The unit of the measures is nanomolar (nM). Values are shown for cells pre-treated with pro-uPA (pro-uPA pre-treat.) and vehicle pre-treated cells (veh. pre-treat.).

TABLE 3
Sequences of isolated scFv's

| #Found | Clone | Class | CDR3 VH | CDR3 VL | FACS (−) | FACS (+) |
|---|---|---|---|---|---|---|
| 200 | 1C1 | A | E/YDPL/F | S/SPSPSA/V | ++ | +(+) |
| | | | (SEQ ID NO: 72) | (SEQ ID NO: 73) | | |
| 1 | 3B6 | A | E/WDPA | S/SMMKTP/V | +(+) | ++ |
| | | | (SEQ ID NO: 22) | (SEQ ID NO: 74) | | |
| 5 | 2B10 | B | K/RFGL/F | S/LPLNST/V | − | − |
| | | | (SEQ ID NO: 75) | (SEQ ID NO: 76) | | |
| 5 | 2A3 | B | K/RWGR/F | S/EPYLT/V | − | − |
| | | | (SEQ ID NO: 77) | (SEQ ID NO: 78) | | |
| 2 | 1G5 | C | K/SKGLPY/F | S/HSLNPP/V | +++ | +++ |
| | | | (SEQ ID NO: 79) | (SEQ ID NO: 80) | | |
| 2 | 3D9 | C | K/SKGVPY/F | S/QHRAQP/V | +++ | +++ |
| | | | (SEQ ID NO: 81) | (SEQ ID NO: 82) | | |
| 1 | 2H10 | C | K/SQGLPY/F | S/ADQAPV/V | +++ | +++ |
| | | | (SEQ ID NO: 83) | (SEQ ID NO: 84) | | |
| 1 | 3C10 | C | K/TKGLPH/F | S/AATGGP/V | +++ | ++(+) |
| | | | (SEQ ID NO: 23) | (SEQ ID NO: 85) | | |
| 4 | 1E6 | D | K/VGKN/F | S/WDKVKP/V | − | − |
| | | | (SEQ ID NO: 86) | (SEQ ID NO: 87) | | |
| 1 | 2G5 | D | K/VGRN/F | S/VSNRTP/V | (+) | − |
| | | | (SEQ ID NO: 88) | (SEQ ID NO: 89) | | |
| 1 | 1C5 | D | K/GRFV/F | S/VWPWPR/V | + | + |
| | | | (SEQ ID NO: 90) | (SEQ ID NO: 91) | | |
| 1 | 1C6 | E | K/RGPKS/F | S/MASSRP/V | − | − |
| | | | (SEQ ID NO: 92) | (SEQ ID NO: 93) | | |
| 1 | 1C3 | F | K/VFAHG/F | S/LPPLHP/V | − | − |
| | | | (SEQ ID NO: 94) | (SEQ ID NO: 95) | | |

Table showing the sequences of the heavy (VH) and light (VL) chain complementarity determining regions 3 (CDR3) of the isolated scFv's numbered according to (Silacci et al., 2005). The sequences between the dashes are the regions hyper-mutated in the phage library. A total of 13 unique sequences were found among the 255 clones analyzed. For each unique sequence the number of clones having this sequence is shown (#Found) together with the name of the representative clone used for biochemical characterization. The unique sequences have been grouped into classes (A to F) based on homology between the sequences. The reactivity with cell surface uPAR (see FIG. 25) in the absence (FACS(−)) and in the presence (FACS(+)) of pro-uPA is presented in arbitrary units representing no (−), low (+), intermediate (++) and high (+++) reactivity. Conserved residues within each class are underlined.

TABLE 4
Anti^{mGFD} muPAR-hFc monoclonal antibodies

| Mouse | Clone | Subclone | Isotype |
|---|---|---|---|
| #1676 | NE43 | NE43-3 | IgG1 κ |
| #1676 | NM23 | NM23-1 | IgG1 κ |
| #1679 | OMD4 | OMD4-6 | IgA |
| #1679 | OOF12 | OOF12-3 | IgG1 κ |

Table indicating the mouse ID, clone number, subclone number, and the isotype of the anti-^{mGFD}muPAR-Fc monoclonal antibodies.

### Sequences

Wild-type human uPAR
(SEQ ID NO: 58)
MGHPPLLPLLLLLHTCVPASWGLRCMQCKTNGDCRVEECALGQDLCRTT
IVRLWEEGEELELVEKSCTHSEKTNRTLSYRTGLKITSLTEVVCGLDLC
NQGNSGRAVTYSRSRYLECISCGSSDMSCERGRHQSLQCRSPEEQCLDV
VTHWIQEGEEGRPKDDRHLRGCGYLPGCPGSNGFHNNDTFHFLKCCNTT
KCNEGPILELENLPQNGRQCYSCKGNSTHGCSSEETFLIDCRGPMNQCL
VATGTHEPKNQSYMVRGCATASMCQHAHLGDAFSMNHIDVSCCTKSGCN
HPDLDVQYRSGAAPQPGPAHLSLTITLLMTARLWGGTLLWT

Amino acid sequence of wild-type human uPAR with the signal peptide (met⁻²² Gly⁻¹) in cursive, a C-terminal peptide (Ala²⁸⁴-Thr³¹³) removed during synthesis upon addition of the glycolipid membrane anchor attached to Gly²⁸³ in cursive and underlined, and the mature protein (Leu¹-Gly²⁸³) in bold.

The minimal essential region of human uPAR (TrP³²-Tyr⁹²) expected to be required for the generation of the antibodies described herein is shown in bold and underlined, corresponding to aa 32-92 of SEQ ID NO:1.

Wild-type mouse uPAR
(SEQ ID NO: 96)
MGLPRRLLLLLLLATTCVPASQGLQCMQCESNQSCLVEECALGQDLCRT
TVLREWQDDRELEVVTRGCAHSEKTNRTMSYRMGSMIISLTETVCATNL
CNRPRPGARGRAFPQGRYLECASCTSLDQSCERGREQSLQCRYPTEHCI
EVVTLQSTERSLKDQDYTRGCGSLPGCPGTAGFHSNQTFHFLKCCNYTH
CNGGPVLDLQSFPPNGFQCYSCEGNNTLGCSSEEASLINCRGPMNQCLV
ATGLDVLGNRSYTVRGCATASWCQGSHVADSFPTHLNVSVSCCHGSGCN
SPTGGAPRPGPAQLSLIASLLLTLGLWGVLLWT

Amino acid sequence of wild-type mouse uPAR with the signal peptide (Met⁻²³-Gly⁻¹) in cursive, a C-terminal peptide (Gly²⁷⁶-Thr³⁹⁴) removed during synthesis upon addition of the glycolipid membrane anchor attached to Gly²⁷⁵ in cursive and underlined, and the mature protein (Leu¹-Gly²⁷⁵) in bold.

The minimal essential region of mouse uPAR (TrP³²-Tyr⁹³) expected to be required for the generation of the antibodies described herein is shown in bold and underlined, corresponding to aa 32-93 of SEQ ID NO:2.

Wild-type mature mouse uPAR
(SEQ ID NO: 2)
LQCMQCESNQSCLVEECALGQDLCRTTVLREWQDDRELEVVTRGCAHSE
KTNRTMSYRMGSMIISLTETVCATNLCNRPRPGARGRAFPQGRYLECAS
CTSLDQSCERGREQSLQCRYPTEHCIEVVTLQSTERSLKDQDYTRGCGS
LPGCPGTAGFHSNQTFHFLKCCNYTHCNGGPVLDLQSFPPNGFQCYSCE
GNNTLGCSSEEASLINCRGPMNQCLVATGLDVLGNRSYTVRGCATASWC
QGSHVADSFPTHLNVSVSCCHGSGCNSPTG
Sequence 1: uPAR-hFc
(SEQ ID NO: 14)
LRCMQCKTNGDCRVEECALGQDLCRTTIVRLWEEGEELELVEKSCTHSE
KTNRTLSYRTGLKITSLTEVVCGLDLCNQGNSGRAVTYSRSRYLECISC
GSSDMSCERGRHQSLQCRSPEEQCLDVVTHWIQEGEEGRPKDDRHLRGC
GYLPGCPGSNGFHNNDTFHFLKCCNTTKCNEGPILELENLPQNGRQCYS
CKGNSTHGCSSEETFLIDCRGPMNQCLVATGTHEPKNQSYMVRGCATAS
MCQHAHLGDAFSMNHIDVSCCTKSGCNHPDLDLEVLFQGPLELEVLFQG
PIEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTK
NQVSLTCLVKGFYPSDIAVEWESNGQPENNYKATPPVLDSDGSFFLYSK
LTVDKSRWQQGNVFSCSVMHEALHNHYTQKSLSLSPGK

uPAR residues (Sequence 1A corresponding to aa. 1-277 of SEQ ID NO:1) are shown in plain text, the linker region (Sequence 1B (SEQ ID NO: 9)) is underlined and the C-terminal human Fc-tag (Sequence 1C (SEQ ID NO: 5)) is in cursive.

Sequence 1A: uPAR residues 1 to 277, corresponding
to aa. 1-277 of SEQ ID NO: 1
LRCMQCKTNGDCRVEECALGQDLCRTTIVRLWEEGEELELVEKSCTHSEK
TNRTLSYRTGLKITSLTEVVCGLDLCNQGNSGRAVTYSRSRYLECISCGS
SDMSCERGRHQSLQCRSPEEQCLDVVTHWIQEGEEGRPKDDRHLRGCGYL
PGCPGSNGFHNNDTFHFLKCCNTTKCNEGPILELENLPQNGRQCYSCKGN
STHGCSSEETFLIDCRGPMNQCLVATGTHEPKNQSYMVRGCATASMCQHA
HLGDAFSMNHIDVSCCTKSGCNHPDLD

The expected minimal functional sequence (residues 3 to 271) is underlined.

Sequence 1B: Linker
(SEQ ID NO: 9)
Sequence: LEVLFQGPLELEVLFQGPIE

There are no predicted specific requirements to the length or sequence of this linker. Possibly it may be entirely omitted.

Sequence 1C: Human IgG hinge and constant region
(hFc)
(SEQ ID NO: 5)
PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLTCLVKGFYPSDIAVEWESNGQPENNYKATPPVLDSDGSFFLYSKLTV
DKSRWQQGNVFSCSVMHEALHNHYTQKSLSLSPGK

Similar sequences from other immunoglobulin types and/or species are likely to work equally well as long as they form dimers or oligomers.

Sequence 2: uPAR-mFc
(SEQ ID NO: 15)
LRCMQCKTNGDCRVEECALGQDLCRTTIVRLWEEGEELELVEKSCTHSE
KTNRTLSYRTGLKITSLTEVVCGLDLCNQGNSGRAVTYSRSRYLECISC
GSSDMSCERGRHQSLQCRSPEEQCLDVVTHWIQEGEEGRPKDDRHLRGC
GYLPGCPGSNGFHNNDTFHFLKCCNTTKCNEGPILELENLPQNGRQCYS
CKGNSTHGCSSEETFLIDCRGPMNQCLVATGTHEPKNQSYMVRGCATAS
MCQHAHLGDAFSMNHIDVSCCTKSGCNHPDLDLEVLFQGPLEAGAGPRD
CGCKPCICTVPEVSSVFIFPPKPKDVLTITLTPKVTCVVVDISKDDPEV
QFSWFVDDVEVHTAQTKPREEQFNSTFRSVSELPIMHQDWLNGKEFKCR
VNSAAFPAPIEKTISKTKGRPKAPQVYTIPPPKEQMAKDKVSLTCMITD
FFPEDITVEWQWNGQPAENYKNTQPIMDTDGSYFVYSKLNVQKSNWEAG
NTFTCSVLHEGLHNHHTEKSLSHSPGK

The mature sequence of uPAR-mFc is composed of human uPAR residues 1-277 (Sequence 1A corresponding to aa. 1-277 of SEQ ID NO:1) shown in plain text, a LEVLFQGPLEAGAG linker is underlined (Sequence 2A (SEQ ID NO: 10)) and the hinge and constant region of a mouse IgG1 (Sequence 2B (SEQ ID NO: 6)) is shown in cursive.

Sequence 2A: Linker.
(SEQ ID NO: 10)
LEVLFQGPLEAGAG

There are no predicted specific requirements to the length or sequence of this linker. Possibly it may be entirely omitted.

Sequence 2B: Mouse IgG hinge and constant region
(mFc).
(SEQ ID NO: 6)
PRDCGCKPCICTVPEVSSVFIFPPKPKDVLTITLTPKVTCVVVDISKDD
PEVQFSWFVDDVEVHTAQTKPREEQFNSTFRSVSELPIMHQDWLNGKEF
KCRVNSAAFPAPIEKTISKTKGRPKAPQVYTIPPPKEQMAKDKVSLTCM
ITDFFPEDITVEWQWNGQPAENYKNTQPIMDTDGSYFVYSKLNVQKSNW
EAGNTFTCSVLHEGLHNHHTEKSLSHSPGK

Sequence of the mouse IgG hinge and constant region (mFc) tag consisting residues 216-441 of a mouse immunoglobulin heavy chain (numbered according to (Adetugbo, 1978)). Similar sequences from other immunoglobulin types and/or species are likely to work equally well as long as they form dimers or oligomers.

Sequence 3: uPARmyc
(SEQ ID NO: 26)
LRCMQCKTNGDCRVEECALGQDLCRTTIVRLWEEGEELELVEKSCTHSE
KTNRTLSYRTGLKITSLTEVVCGLDLCNQGNSGRAVTYSRSRYLECISC
GSSDMSCERGRHQSLQCRSPEEQCLDVVTHWIQEGEEGRPKDDRHLRGC
GYLPGCPGSNGFHNNDTFHFLKCCNTTKCNEGPILELENLPQNGRQCYS
CKGNSTHGCSSEETFLIDCRGPMNQCLVATGTHEPKNQSYMVRGCATAS
MCQHAHLGDAFSMNHIDVSCCTKSGCNHPGGEQKLISEEDL

The polypeptide sequence of soluble human uPAR (residues 1 to 274, Sequence 3A corresponding to aa. 1-274 of SEQ ID NO: 1) is shown in plain text and the C-terminal myc-tag (GGEQKLISEEDL, Sequence 3B corresponding to SEQ ID NO: 27) in cursive.

Sequence 3A: uPAR residues 1-274, corresponding
to aa. 1-274 of SEQ ID NO: 1)
LRCMQCKTNGDCRVEECALGQDLCRTTIVRLWEEGEELELVEKSCTHSE
KTNRTLSYRTGLKITSLTEVVCGLDLCNQGNSGRAVTYSRSRYLECISC
GSSDMSCERGRHQSLQCRSPEEQCLDVVTHWIQEGEEGRPKDDRHLRGC
GYLPGCPGSNGFHNNDTFHFLKCCNTTKCNEGPILELENLPQNGRQCYS
CKGNSTHGCSSEETFLIDCRGPMNQCLVATGTHEPKNQSYMVRGCATAS
MCQHAHLGDAFSMNHIDVSCCTKSGCNHP

The expected minimal functional sequence (residues 3 to 271 (aa. 3-271 of SEQ ID NO: 1)) is underlined.

Sequence 3B: myc-tag
(SEQ ID NO: 27)
GGEQKLISEEDL

The sole purpose of this C-terminal tag is for immunological detection and/or purification. The sequence may be eliminated without functional consequences.

Sequence 4: Wild-type mature human uPAR
(SEQ ID NO: 1)
LRCMQCKTNGDCRVEECALGQDLCRTTIVRLWEEGEELELVEKSCTHSE
KTNRTLSYRTGLKITSLTEVVCGLDLCNQGNSGRAVTYSRSRYLECISC
GSSDMSCERGRHQSLQCRSPEEQCLDVVTHWIQEGEEGRPKDDRHLRGC
GYLPGCPGSNGFHNNDTFHFLKCCNTTKCNEGPILELENLPQNGRQCYS
CKGNSTHGCSSEETFLIDCRGPMNQCLVATGTHEPKNQSYMVRGCATAS
MCQHAHLGDAFSMNHIDVSCCTKSGCNHPDLDVQYRSG-
(GPI-anchor)

Mature human uPAR (residues 1-283) is linked to the cell membrane by glycolipid anchor attached to the C-terminal residue (Gly283).

Sequence 5: ^{GFD}UPAR
(SEQ ID NO: 16)
SNELHQVPSNCDCLNGGTCVSNKYFSNIHWCNCPKKFGGQHCEIDKSKG
GAGAAGGLRCMQCKTNGDCRVEECALGQDLCRTTIVRLWEEGEELELVE
KSCTHSEKTNRTLSYRTGLKITSLTEVVCGLDLCNQGNSGRAVTYSRSR
YLECISCGSSDMSCERGRHQSLQCRSPEEQCLDVVTHWIQEGEEGRPKD
DRHLRGCGYLPGCPGSNGFHNNDTFHFLKCCNTTKCNEGPILELENLPQ
NGRQCYSCKGNSTHGCSSEETFLIDCRGPMNQCLVATGTHEPKNQSYMV
RGCATASMCQHAHLGDAFSMNH1DVSCCTKSGCNHPDLDVQYRSG-
(GPI-anchor)

The polypeptide sequence of ^{GFD}uPAR is shown with the GFD-domain of human uPA (residues 1 to 48, Sequence 5A (SEQ ID NO: 3)) in bold, an 8-residue GGAGAAGG linker (Sequence 5B (SEQ ID NO: 7)) is underlined and mature human uPAR (residues 1-283, Sequence 4 (SEQ ID NO: 1)) is shown as plain text. The mature ^{GFD}uPAR polypeptide is tethered to the cell membrane by a GPI-anchor attached on the C-terminal residue of uPAR (Gly283).

Sequence 5A: The growth factor-like domain (GFD)
of human uPA (residues 1 to 48)
(SEQ ID NO: 3)
SNELHQVPSNCDCLNGGTCVSNKYFSNIEWCNCPKKFGGQHCEIDKSK

The predicted minimal sequence is underlined.

Sequence 5B: Linker sequence
(SEQ ID NO: 7)
GGAGAAGG

The length and sequence of this linker is likely to affect the biochemical properties of ^{GFD}uPAR as it may determine if the binding of the GFD-domain to the uPAR-domains of the chimera occurs in cis and/or in trans (see FIG. 8) Experimentally, linkers 5, 8, 16 and 20 residues long all work well (see FIG. 7) suggesting that the length and amino acid composition of this linker is very flexible.

Sequence 6: ^{GFD}uPARmyc
(SEQ ID NO: 28)
SNELHQVPSNCDCLNGGTCVSNKYFSNIHWCNCPKKFGGQHCEIDKSKG
GAGAAGGLRCMQCKTNGDCRVEECALGQDLCRTTIVRLWEEGEELELVE
KSCTHSEKTNRTLSYRTGLKITSLTEVVCGLDLCNQGNSGRAVTYSRSR
YLECISCGSSDMSCERGRHQSLQCRSPEEQCLDVVTHWIQEGEEGRPKD
DRHLRGCGYLPGCPGSNGFHNNDTFHFLKCCNTTKCNEGPILELENLPQ
NGRQCYSCKGNSTHGCSSEETFLIDCRGPMNQCLVATGTHEPKNQSYMV
RGCATASMCQHAHLGDAFSMNHIDVSCCTKSGCNHPGGEQKLISEEDL

The polypeptide sequence of ^{GFD}uPARmyc is shown with the GFD-domain of human uPA (residues 1 to 48, Sequence 5A (SEQ ID NO: 3)) in bold, an 8-residue GGAGAAGG linker (Sequence 5B (SEQ ID NO: 7)) is underlined, human uPAR residues 1-274 (Sequence 3A corresponding to aa. 1-274 of SEQ ID NO: 1)) is shown as plain text and a C-terminal myc-tag (Sequence 3B (SEQ ID NO: 27)) in cursive.

Sequence 7: ^{GFD}uPAR-hFc
(SEQ ID NO: 12)
SNELHQVPSNCDCLNGGTCVSNKYFSNIHWCNCPKKFGGQHCEIDKSKG
GAGAAGGLRCMQCKTNGDCRVEECALGQDLCRTTIVRLWEEGEELELVE
KSCTHSEKTNRTLSYRTGLKITSLTEVVCGLDLCNQGNSGRAVTYSRSR
YLECISCGSSDMSCERGRHQSLQCRSPEEQCLDVVTHWIQEGEEGRPKD
DRHLRGCGYLPGCPGSNGFHNNDTFHFLKCCNTTKCNEGPILELENLPQ
NGRQCYSCKGNSTHGCSSEETFLIDCRGPMNQCLVATGTHEPKNQSYMV
RGCATASMCQHAHLGDAFSMNHIDVSCCTKSGCNHPDLDLEVLFQGPLE
LEVLFQGPIEPKSCDKTHTCPPCPAPELLGGPSVFLFPPKPKDTLMISR
TPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPP
SRDELTKNQVSLTCLVKGFYPSDIAVEWESNGQPENNYKATPPVLDSDG
SFFLYSKLTVDKSRWQQGNVFSCSVMHEALHNHYTQKSLSLSPG

The ^{GFD}uPAR-hFc polypeptide is composed of the GFD domain (Sequence 5A (SEQ ID NO: 3)) shown in bold, a linker (Sequence 5B (SEQ ID NO: 7)) is underlined, uPAR residues 1-277 (Sequence 1A corresponding to aa. 1-277 of SEQ ID NO: 1)) in plain text, a linker (Sequence 1B (SEQ ID NO: 9)) in underlined cursive and a human Fc-tag (Sequence 1C (SEQ ID NO: 5)) in cursive.

Sequence 8: ^{GFD}uPAR-mFc
(SEQ ID NO: 13)
SNELHQVPSNCDCLNGGTCVSNKYFSNIHWCNCPKKFGGQHCEIDKSKG
GAGAAGGLRCMQCKTNGDCRVEECALGQDLCRTTIVRLWEEGEELELVE
KSCTHSEKTNRTLSYRTGLKITSLTEVVCGLDLCNQGNSGRAVTYSRSR
YLECISCGSSDMSCERGRHQSLQCRSPEEQCLDVVTHWIQEGEEGRPKD
DRHLRGCGYLPGCPGSNGFHNNDTFHFLKCCNTTKCNEGPILELENLPQ
NGRQCYSCKGNSTHGCSSEETFLIDCRGPMNQCLVATGTHEPKNQSYMV
RGCATASMCQHAHLGDAFSMNHIDVSCCTKSGCNHPDLDLEVLFQGPLE
AGAGPRDCGCKPCICTVPEVSSVFIFPPKPKDVLTITLTPKVTCVVVDI
SKDDPEVQFSWFVDDVEVHTAQTKPREEQFNSTFRSVSELPIMHQDWLN
GKEFKCRVNSAAFPAPIEKTISKTKGRPKAPQVYTIPPPKEQMAKDKVS
LTCMITDFFPEDITVEWQWNGQPAENYKNTQPIMDTDGSYFVYSKLNVQ
KSNWEAGNTFTCSVLHEGLHNHHTEKSLSHSPGK

The ^{GFD}uPAR-mFc polypeptide is composed of the GFD domain (Sequence 5A (SEQ ID NO: 3)) shown in bold, a linker (Sequence 5B (SEQ ID NO: 7)) is underlined, uPAR residues 1-277 (Sequence 1A corresponding to aa. 1-277 of SEQ ID NO: 1)) in plain text, a linker (Sequence 2A (SEQ ID NO: 10)) in underlined cursive and a mouse Fc-tag (Sequence 2B (SEQ ID NO: 6)) in cursive.

Sequence 9: ^{mGFD}muPAR-Fc
(SEQ ID NO: 17)
GSVLGAPDESNCGCQNGGVCVSYKYFSRIRRCSCPRKFQGEHCEIDASK
GGAGAAGGLQCMQCESNQSCLVEECALGQDLCRTTVLREWQDDRELEVV
TRGCAHSEKTNRTMSYRMGSMIISLTETVCATNLCNRPRPGARGRAFPQ
GRYLECASCTSLDQSCERGREQSLQCRYPTEHCIEVVTLQSTERSLKDE
DYTRGCGSLPGCPGTAGFHSNQTFHFLKCCNYTHCNGGPVLDLQSFPPN
GFQCYSCEGNNTLGCSSEEASLINCRGPMNQCLVATGLDVLGNRSYTVR
GCATASWCQGSHVADSFPTHLNVSVSCCHGSGCNSPVELEVLFQGPIEP
KSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVS
LTCLVKGFYPSDIAVEWESNGQPENNYKATPPVLDSDGSFFLYSKLTVD
KSRWQQGNVFSCSVMHEALHNHYTQKSLSLSPGK

The ^{mGFD}muPAR-Fc polypeptide is composed of the mouse GFD domain (Sequence 9A (SEQ ID NO: 4)) shown in bold, a linker (Sequence 9B (SEQ ID NO: 8)) is underlined, mouse uPAR residues 1-273 (Sequence 9C corresponding to aa. 1-273 of SEQ ID NO: 2) in plain text, a linker (Sequence 9D (SEQ ID NO: 11)) in underlined cursive and a human Fc-tag (Sequence 1C (SEQ ID NO: 5)) in cursive.

Sequence 9A: The growth factor-like domain (GFD)
of mouse uPA (residues 1 to 49)
(SEQ ID NO: 4)
GSVLGAPDESNCGCQNGGVCVSYKYFSRIRRCSCPRKFQGEHCEIDASK

The predicted minimal sequence (residues 12-43) is underlined.

Sequence 9B: Linker sequence
(SEQ ID NO: 8)
GGAGAAGG

The length and sequence of this linker is likely to affect the biochemical properties of ^{mGFD}muPAR-Fc. Experimentally, linkers 5, 8, 16 and 20 residues long all work well in the human variant (see FIG. 7), suggesting that in practice the length and amino acid composition of this linker is very flexible.

Sequence 9C: mouse uPAR residues 1 to 273
corresponding to aa. 1-273 of SEQ ID NO: 2
LQCMQCESNQSCLVEECALGQDLCRTTVLREWQDDRELEVVTRGCAHSE
KTNRTMSYRMGSMIISLTETVCATNLCNRPRPGARGRAFPQGRYLECAS
CTSLDQSCERGREQSLQCRYPTEHCIEVVTLQSTERSLKDEDYTRGCGS
LPGCPGTAGFHSNQTFHFLKCCNYTHCNGGPVLDLQSFPPNGFQCYSCE
GNNTLGCSSEEASLINCRGPMNQCLVATGLDVLGNRSYTVRGCATASWC
QGSHVADSFPTHLNVSVSCCHGSGCNSP

The expected minimal functional sequence (residues 3 to 270) is underlined.

Sequence 9D: linker
(SEQ ID NO: 11)
VELEVLFQGPIE

There are no predicted specific requirements to the length or amino acid composition of this sequence.

### REFERENCES

Adetugbo, K. (1978). Evolution of immunoglobulin subclasses. Primary structure of a murine myeloma gammal chain. J Biol Chem 253, 6068-6075.
Caiolfa, V. R., Zamai, M., Malengo, G., Andolfo, A., Madsen, C. D., Sutin, J., Digman, M. A., Gratton, E., Blasi, F., and Sidenius, N. (2007). Monomer dimer dynamics and distribution of GPI-anchored uPAR are determined by cell surface protein assemblies. J Cell Biol 179, 1067-1082.
Deng, G., Curriden, S. A., Wang, S., Rosenberg, S., and Loskutoff, D. J. (1996). Is plasminogen activator inhibitor-1 the molecular switch that governs urokinase receptor-mediated cell adhesion and release? J Cell Biol 134, 1563-1571.
Galfre, G., Howe, S. C., Milstein, C., Butcher, G. W., and Howard, J. C. (1977). Antibodies to major histocompatibility antigens produced by hybrid cell lines. Nature 266, 550-552.
Gardsvoll, H., Hansen, L. V., Jorgensen, T. J., and Ploug, M. (2007). A new tagging system for production of recombinant proteins in Drosophila S2 cells using the third domain of the urokinase receptor. Protein Expr Purif 52, 384-394.
Gardsvoll, H., and Ploug, M. (2007). Mapping of the Vitronectin-binding Site on the Urokinase Receptor: INVOLVEMENT OF A COHERENT RECEPTOR INTERFACE CONSISTING OF RESIDUES FROM BOTH DOMAIN I AND THE FLANKING INTERDOMAIN LINKER REGION. J Biol Chem 282, 13561-13572.
Huai, Q., Zhou, A., Lin, L., Mazar, A. P., Parry, G. C., Callahan, J., Shaw, D. E., Furie, B., Furie, B. C., and Huang, M. (2008). Crystal structures of two human vitronectin, urokinase and urokinase receptor complexes. Nat Struct Mol Biol 15, 422-423.
Kenny, H. A., Leonhardt, P., Ladanyi, A., Yamada, S. D., Montag, A., Im, H. K., Jagadeeswaran, S., Shaw, D. E., Mazar, A. P., and Lengyel, E. (2010). Targeting the urokinase plasminogen activator receptor inhibits ovarian cancer metastasis. Clin Cancer Res 17, 459-471.
Madsen, C. D., Ferraris, G. M., Andolfo, A., Cunningham, O., and Sidenius, N. (2007). uPAR-induced cell adhesion and migration: vitronectin provides the key. J Cell Biol 177, 927-939.
Rabbani, S. A., Ateeq, B., Arakelian, A., Valentino, M. L., Shaw, D. E., Dauffenbach, L. M., Kerfoot, C. A., and Mazar, A. P. (2010). An anti-urokinase plasminogen activator receptor antibody (ATN-658) blocks prostate cancer invasion, migration, growth, and experimental skeletal metastasis in vitro and in vivo. Neoplasia 12, 778-788.
Sidenius, N., Andolfo, A., Fesce, R., and Blasi, F. (2002). Urokinase regulates vitronectin binding by controlling urokinase receptor oligomerization. J Biol Chem 277, 27982-27990.
Sidenius, N., and Blasi, F. (2000). Domain 1 of the urokinase receptor (uPAR) is required for uPAR-mediated cell binding to vitronectin. FEBS Lett 470, 40-46.
Silacci, M., Brack, S., Schirru, G., Marlind, J., Ettorre, A., Merlo, A., Viti, F., and Neri, D. (2005). Design, construction, and characterization of a large synthetic human antibody phage display library. Proteomics 5, 2340-2350.
Tjwa, M., Sidenius, N., Moura, R., Jansen, S., Theunissen, K., Andolfo, A., De Mol, M., Dewerchin, M., Moons, L., Blasi, F., et al. (2009). Membrane-anchored uPAR regulates the proliferation, marrow pool size, engraftment, and mobilization of mouse hematopoietic stem/progenitor cells. J Clin Invest 119, 1008-1018.
Van Buren, G., 2nd, Gray, M. J., Dallas, N. A., Xia, L., Lim, S. J., Fan, F., Mazar, A. P., and Ellis, L. M. (2009). Targeting the urokinase plasminogen activator receptor with a monoclonal antibody impairs the growth of human colorectal cancer in the liver. Cancer 115, 3360-3368.
Wang, Z., Raifu, M., Howard, M., Smith, L., Hansen, D., Goldsby, R., and Ratner, D. (2000). Universal PCR amplification of mouse immunoglobulin gene variable regions: the design of degenerate primers and an assessment of the effect of DNA polymerase 3′ to 5′ exonuclease activity. J Immunol Methods 233, 167-177.

1. An urokinase plasminogen activator receptor (uPAR) variant molecule having an increased VN-binding activity with respect to the wild type molecule.

2. The uPAR variant molecule according to claim 1 comprising a wild type uPAR amino acid sequence linked to:
a) a growth factor-like domain (GFD) sequence of uPA at the N-terminal of the wild type uPAR sequence, and/or
b) a chain of the Fc region of an antibody molecule at the C-terminal of the wild type uPAR sequence,
wherein if said chain of the Fc region is present, the uPAR variant molecule is a dimer.

3. The uPAR variant molecule according to claim 2 wherein the wild type uPAR sequence comprises a sequence consisting essentially of the aa. 32-92 of mature huPAR of Seq ID NO: 1 or a sequence consisting essentially of the aa. 32-93 of mature muPAR of Seq ID NO: 2 or a polypeptide encoded by the correspondent regions from an uPAR orthologous gene, or functional mutants or derivatives or analogues thereof.

4. The uPAR variant molecule according to claim 2 wherein the wild type uPAR sequence comprises a sequence consisting essentially of the aa. 3-271 of mature huPAR of Seq ID NO: 1 or a sequence consisting essentially of the aa. 3-270 of mature muPAR of Seq ID NO: 2 or a polypeptide encoded by the correspondent regions from an uPAR orthologous gene, or functional mutants or derivatives or analogues thereof.

5. The uPAR variant molecule according to claim 2 wherein the wild type uPAR sequence comprises a sequence consisting essentially of the aa. 1-277 of mature huPAR of Seq ID NO: 1 or a sequence consisting of essentially the aa. 1-273 of mature muPAR of Seq ID NO: 2 or a polypeptide encoded by the correspondent regions from an uPAR orthologous gene, or functional mutants or derivatives or analogues thereof.

6. The uPAR variant molecule according to claim 2 wherein the wild type uPAR sequence comprises a sequence consisting essentially of Seq ID NO: 1 or Seq ID NO: 2 or a polypeptide encoded by the correspondent region from a uPAR orthologous gene, or functional mutants or derivatives or analogues thereof.

7. The uPAR variant molecule according to claim 2, wherein the GFD sequence of uPA comprises a sequence consisting essentially of the aa. 11-42 of the GFD of human uPA of SEQ ID NO: 3 or a sequence consisting essentially of the aa. 12-43 of the GFD of mouse uPA of SEQ ID NO: 4 or a polypeptide encoded by the correspondent region from a GDF orthologous gene, or functional mutants or derivatives or analogues thereof.

8. The uPAR variant molecule according to claim 2, wherein the GFD sequence of uPA consists essentially of the GFD sequence of human uPA of SEQ ID NO: 3 or of the GFD sequence of mouse uPA of SEQ ID NO: 4 or a polypeptide encoded by the correspondent region from a GFD orthologous gene, or functional mutants or derivatives or analogues thereof.

9. The uPAR variant molecule according to claim 2, wherein the chain of the Fc region is of human origin and comprises a sequence consisting essentially of SEQ ID NO: 5 or the chain of the Fc region is of mouse origin and comprises a sequence consisting essentially of SEQ ID NO: 6 or a polypeptide encoded by the correspondent region from a chain of the Fc region orthologous gene, or functional mutants or derivatives or analogues thereof.

10. The uPAR variant molecule according to claim 2 further comprising:
a) a first linker region between the GFD sequence of uPA and the N-terminal of the wild type uPAR sequence, and/or
b) a second linker region between the chain of the Fc region of an antibody molecule and the C-terminal of the wild type uPAR sequence.

11. The uPAR variant molecule according to claim 10 wherein the first linker region consists essentially of the sequence of SEQ ID NO: 7 or SEQ ID NO: 8.

12. The uPAR variant molecule according to claim 10 wherein the second linker region consists essentially of the sequence of SEQ ID NO: 9, SEQ ID NO: 10 or SEQ ID NO: 11.

13. The uPAR variant molecule according to claim 1, comprising a sequence having essentially the sequence of SEQ ID NOs: 12, 13, 14, 15, 16 or 17.

14. A method of obtaining a specific antibody molecule having an antagonist activity of uPAR functions, comprising immunizing a subject with the molecule of claim 1.

15. An antibody, recombinant or synthetic antigen-binding fragments thereof able to bind the urokinase plasminogen activator receptor (uPAR) variants described in claim 1.

16. An antibody, recombinant or synthetic antigen-binding fragments thereof according to claim 15 having an antagonist activity of uPAR functions.

17. An antibody, recombinant or synthetic antigen-binding fragments thereof according to claim 15, able to bind to an epitope of uPAR molecule, said epitope comprising at least one of R89, R91 and Y92 amino acid residues.

18. An antibody, recombinant or synthetic antigen-binding fragments thereof according to claim 15, comprising
at least one heavy chain complementary determining region (CDRH3) amino acid sequence having at least 80% identity to an amino acid sequence selected from the group consisting of: aa. 90-102 of SEQ ID NOs: 18, 19, 20, 21 or 25, aa. 90-101 of SEQ ID NOs: 24, and SEQ ID NOs: 22 or 23, and/or
at least one heavy chain complementary determining region (CDRH2) amino acid sequence having at least 80% identity to an amino acid sequence selected from the group consisting of: aa. 41-57 of SEQ ID NOs: 18, 19, 20, 21, 24 or 25, and/or
at least one heavy chain complementary determining region (CDRH1) amino acid sequence having at least 80% identity to an amino acid sequence selected from the group consisting of: aa. 22-26 of SEQ ID NOs: 18, 19, 20, 21 or 24 and aa. 17-26 of SEQ ID NO: 25.

19. An antibody, recombinant or synthetic antigen-binding fragments thereof according to claim 15, comprising at least one light chain complementary determining region (CDRL3) amino acid sequence having at least 80% identity to an amino acid sequence selected from the group consisting of: aa. 80-87 of SEQ ID NOs: 65, 66, 67, 68 or 69, and SEQ ID NOs: 74 or 85, and/or
at least one light chain complementary determining region (CDRL2) amino acid sequence having at least 80% identity to an amino acid sequence selected from the group consisting of: aa. 41-47 of SEQ ID NOs: 65, 66, 67, 68 and 69, and/or
at least one one light chain complementary determining region (CDRL1) amino acid sequence having at least 80% identity to an amino acid sequence selected from the group consisting of: aa. 15-23 of SEQ ID NOs: 65, 66, 67, 68 and 69.

20. An antibody, recombinant or synthetic antigen-binding fragments thereof according to claim 15, comprising a heavy chain variable region comprising an amino acid sequence having at least 80% identity to an amino acid sequence selected from the group consisting of: SEQ ID NOs: 18, 19, 20, 21, 24 and 25 and/or a light chain variable region comprising an amino acid sequence having at least 80% identity to an amino acid sequence selected from the group consisting of: SEQ ID NOs: 65, 66, 67, 68 and 69.

21. The antibody, recombinant or synthetic antigen-binding fragments thereof according to claim 20, comprising a heavy chain variable region comprising an amino acid sequence having at least 80% identity to an amino acid sequence selected from the group consisting of: SEQ ID NOs: 18, 19, 20, 21 and 24 and a light chain variable region comprising an amino acid sequence having at least 80% identity to an amino acid sequence selected from the group consisting of: SEQ ID NOs: 66, 65, 68, 67 and 69 respectively.

22-24. (canceled)

25. A pharmaceutical composition comprising at least one antibody, recombinant or synthetic antigen-binding fragments thereof of claim 15 and appropriated diluents and/or excipients.

26. A method of treating or preventing cancer in a patient, comprising administering to a subject in need thereof a therapeutically effective amount of an antibody, recombinant or synthetic antigen-binding fragments thereof of claim 15.

27. The method of claim 26, wherein the amount administered is from 1 μg/kg to 15 mg/kg.

28. A method for selecting a recombinant or synthetic antigen-binding fragments of an antibody molecule having an antagonist activity of uPAR functions, comprising selecting phages binding to the molecule of claim 1, from a phage display library.
